(19) United States
(12) Patent Application Publication
(10) Pub. No.: US 2017/0249445 A1
(43) Pub. Date: Aug. 31, 2017

(54) PORTABLE DEVICES AND METHODS FOR MEASURING NUTRITIONAL INTAKE

(71) Applicant: BLACKTREE FITNESS TECHNOLOGIES INC., Kitchener (CA)

(72) Inventors: Emmanuel Jesse DEVRIES, Kitchener (CA); Naman KUMAR, Kitchener (CA); Deepak Kumar RANGI, Kitchener (CA)

(73) Assignee: Blacktree Fitness Technologies Inc., Kitcher (CA)

(21) Appl. No.: 15/510,825

(22) PCT Filed: Sep. 11, 2015
(86) PCT No.: PCT/IB2015/056997

§ 371 Date: Mar. 13, 2017

Related U.S. Application Data
(60) Provisional application No. 62/049,674, filed on Sep. 12, 2014.
(60) Provisional application No. 62/087,683, filed on Dec. 4, 2014.

(51) Int. Cl. G06F 19/00; A61B 5/024; A61B 5/00; G09B 19/00

(52) U.S. Cl. CPC G06F 19/3475; G09B 19/0092; A61B 5/02416; A61B 5/742; A61B 5/6804; G06F 19/322; G06F 19/3437

(57) ABSTRACT

A system for monitoring nutritional intake is described. The system includes a wearable housing configured for releasable attachment to a user; a biosensor supported by the wearable housing for disposition adjacent to a blood vessel; the biosensor configured to collect pulse profile data; an output device; and a processing circuit connected to the biosensor and the output device. The processing circuit is configured to: receive the pulse profile data from the biosensor; generate a nutritional intake value from the received pulse profile data; and control an output device to output the nutritional intake value.

### CROSS-REFERENCE TO RELATED APPLICATIONS

This application claims priority to U.S. Provisional Patent Application No. 62/049,674, filed Sep. 12, 2014, and to U.S. Provisional Patent Application No. 62/087,683, filed Dec. 4, 2014, the contents of which are incorporated herein by reference.

### FIELD

The present specification relates generally to biosensors, signal processing, machine learning, physiology and nutritional science, and more particularly relates to various portable devices and methods for measuring nutritional intake.

### BACKGROUND

As portable devices and biosensor technology matures, it has become possible to embed various sensors in wearable devices. As a result, fitness-related products such as heart-rate monitors and the like have become readily available. The derivation of more complex health-related data from biosensors remains problematic, however. The high degree of variability between individuals, in terms of both physiology and behaviour, renders the computation of meaningful health-related data in a repeatable manner difficult and inefficient. Such difficulties can be addressed to a certain extent by the use of additional biosensors and collection techniques. However, such solutions introduce further difficulties, including costly sensor implementations and reduced user compliance.

### SUMMARY

A portable monitoring device is provided for attachment to a user's body. The device employs sensors to gather data with which various metrics, including caloric intake due to ingestion of food by the user, are calculated and presented as output to either or both of the user and other devices.

### BRIEF DESCRIPTION OF THE DRAWINGS

Various embodiments according to the present specification will now be described, with reference to the enclosed Figures, in which:

FIG. 1 is a block diagram representation of an exemplary portable monitoring device, according to an embodiment;

FIG. 2 is a block diagram representation of an exemplary portable monitoring device, according to an embodiment;

FIG. 3 is a block diagram representation of an exemplary portable monitoring device, according to an embodiment;

FIG. 4 is a block diagram representation of an exemplary portable monitoring device, according to an embodiment;

FIG. 5 is a block diagram representation of an exemplary portable monitoring device, according to an embodiment;

FIG. 6 is a block diagram representation of an exemplary portable monitoring device, according to an embodiment;

FIG. 7 is a block diagram representation of processing circuitry to calculate the caloric intake of the user based on sensor data;

FIG. 8 is a graph illustrating exemplary pulse profile signal;

FIG. 9 is a flowchart representing an exemplary process of calculating nutrition-related metrics based on certain sensor data, according to an embodiment;

FIG. 10 is a flowchart representing an exemplary process of calculating features based on certain sensor data, according to an embodiment;

FIG. 11 is a flowchart representing an exemplary process of calculating time-pooled harmonic features based on pulse profile sensor data, according to an embodiment;

FIG. 12 is a flowchart representing an exemplary process of preprocessing pulse profile sensor data, according to an embodiment;

FIG. 13 is a flowchart representing an exemplary process of calculating per-beat harmonic features based on preprocessed pulse profile sensor data, according to an embodiment;

FIG. 14 is a diagram illustrating exemplary time windows with respect to the start of the meal to be used for calculating time-pooled features, according to an embodiment;

FIG. 15 is a flowchart representing an exemplary training process for determining the processing chain configuration (regression model parameters, hyperparameters, etc.) given a dataset;

FIG. 16 is a flowchart representing an exemplary process of preprocessing pulse profile sensor data, according to an embodiment;

FIG. 17 is a graph illustrating an exemplary calculation of the Incremental Area Under the Curve of a given parameter of feature with respect to time

FIG. 18 is a flowchart representing an exemplary process of applying Unsupervised Feature Learning (UFL) to specify/calculate suitable features, according to an embodiment;

FIG. 19 is a flowchart representing an exemplary process of calculating nutrition-related metrics based on certain sensor data, according to an embodiment;

FIG. 20 is a scatter plot of typical data generated by the preferred embodiment, showing caloric intake predictions for a single user, with predictions (horizontal axis) against actual values (vertical axis), and a line for ideal predictions for comparison; each datapoint is a single meal (145 datapoints in total);

FIG. 21 is an error histogram of typical data generated by the preferred embodiment, showing caloric intake predictions for a single user; error (=prediction−actual) is on the horizontal axis, while the vertical axis is the number of meals in each error bin (145 meals total);

FIG. 22 is a block diagram representation of an exemplary portable monitoring device, according to an embodiment;

FIG. 23 is a side perspective view of an exemplary physical configuration of a portable monitoring device according to an embodiment;

FIG. 24 is a top perspective view of an exemplary physical configuration of a portable monitoring device according to an embodiment;

FIG. 25 is a block diagram representation of exemplary portable monitoring devices, according to an embodiment.

### DETAILED DESCRIPTION OF THE EMBODIMENTS

The present specification is directed to portable monitoring devices, and methods of operating and controlling same, which monitor and calculate nutrition-related metrics (such as caloric intake) due to the ingestion of food. The portable monitoring devices can comprise at least one of a pulse profile sensor and a physiological and/or environmental sensor, as well as processing circuitry configured to calculate caloric intake and/or other nutrition-related metrics.

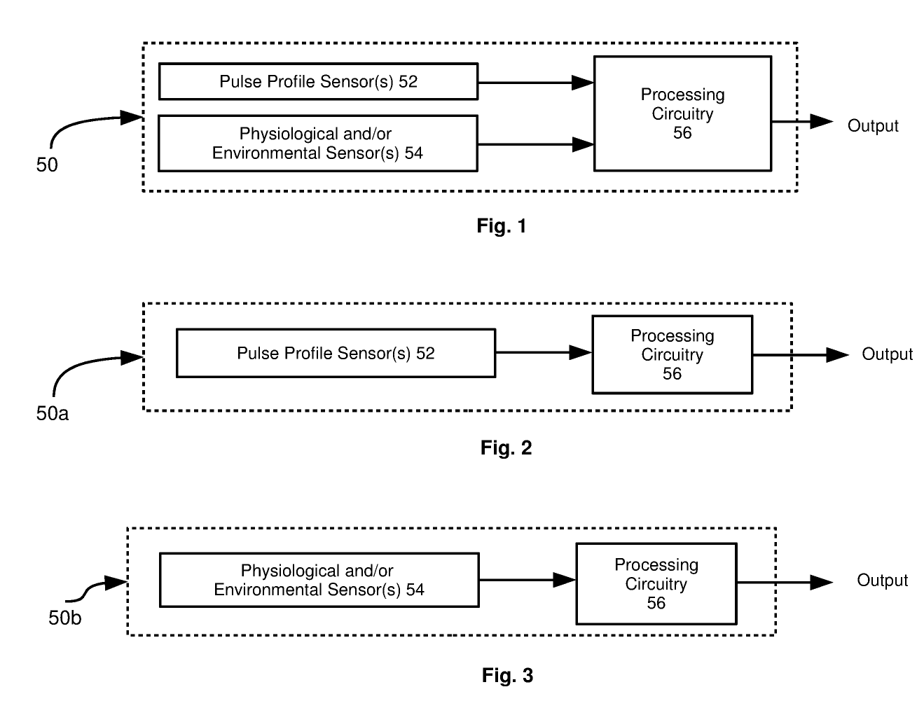

Referring now to FIG. 1, in a first, presently preferred embodiment there is provided a portable monitoring device 50 comprising one or more pulse profile sensor(s) 52, one or more physiological and/or environmental sensor(s) 54, all of which generate outputs that are fed as inputs into a processing circuitry 56.

Referring now to FIG. 2, in a second embodiment there is provided a portable monitoring device 50a (which is a variation on device 50), comprising one or more pulse profile sensor(s) 52 which generate an outputs that are fed as inputs into a processing circuitry 56.

Referring now to FIG. 3, in a third embodiment there is provided a portable monitoring device 50b (which is a variation on device 50), comprising one or more physiological and/or environmental sensor(s) 54 which generate outputs that are fed as inputs into a processing circuitry 56.

Referring now to FIG. 4, in a fourth embodiment there is provided a portable monitoring device 50c, (which is a variation on device 50) comprising one or more pulse profile sensor(s) 52 and one or more physiological and/or environmental sensor(s) 54, which generate outputs that are fed as inputs into a processing circuitry 56. In this embodiment, a user interface 58 can make information received from the processing circuitry 56 available to the user, and can make information received from the user available to the processing circuitry 56. For example, the user interface 58 can comprise a screen (for example, liquid crystal display based or organic light-emitting diode based), and/or button(s), and/or vibration sensor (for example, piezoelectric based or based on an accelerometer or motion sensor), and/or touch sensor(s), and/or gesture sensor(s) (e.g. based on motion sensor(s) and/or EMG sensor(s)), and/or optical indicator(s) (e.g. based on LEDs), and/or vibration motor, and/or speaker, and/or microphone (e.g. with voice recognition techniques). For example, a motion sensor with tapping recognition can be used with an advantage of reducing the need for less reliable and/or harder to integrate mechanical input devices; specific tapping gestures can be recognized e.g. single tap, double tap, etc. For example, proximity detection can be used to determine if portable device 50 is being worn by the user e.g. one or more infrared proximity sensor(s) can be used; furthermore, these proximity sensor(s) can be integrated with pulse profile sensor(s), e.g. in the case of PPG.

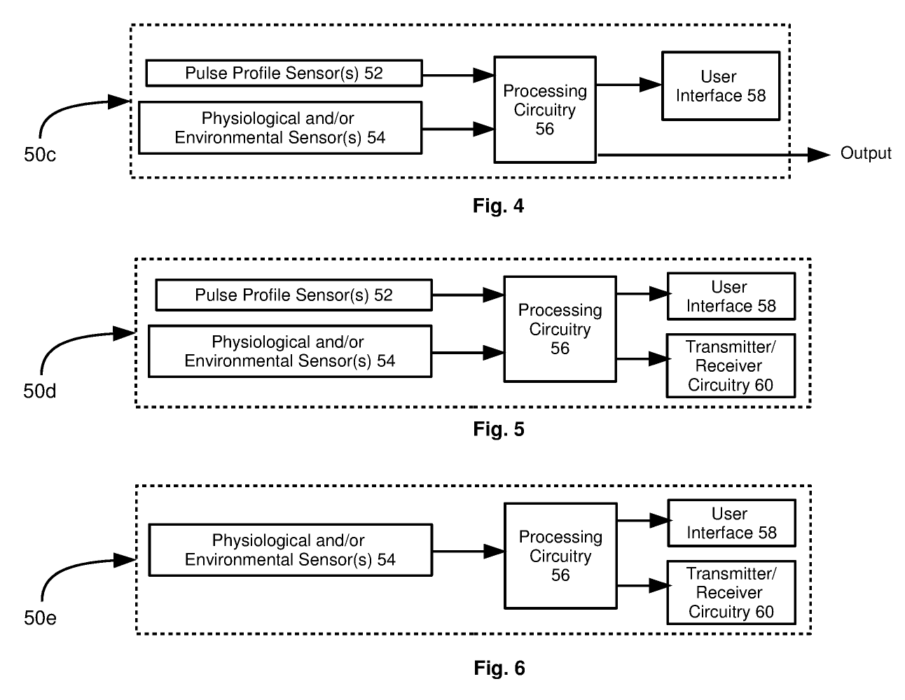

Referring now to FIG. 5, in a fifth embodiment there is provided a portable monitoring device 50d, (which is a variation on device 50) comprising one or more pulse profile sensor(s) 52 and one or more physiological and/or environmental sensor(s) 54, which generate outputs that are fed as inputs into a processing circuitry 56. In this embodiment, a user interface 58 can make information received from the processing circuitry 56 available to the user, and can make information received from the user available to the processing circuitry 56, and transmitter and/or receiver circuitry 60 can transmit information received from the processing circuitry 56 to an external device, and/or can receive information from an external device and make the information received the external device available to the processing circuitry 56.

Referring now to FIG. 6, in a sixth embodiment there is provided a portable monitoring device 50d, (which is a variation on device 50) comprising one or more physiological and/or environmental sensor(s) 54 which generate outputs that are fed as inputs into a processing circuitry 56. In this embodiment, a user interface 58 can make information received from the processing circuitry 56 available to the user, and can make information received from the user available to the processing circuitry 56, and transmitter and/or receiver circuitry 60 can transmit information received from the processing circuitry 56 to an external device, and/or can receive information from an external device and make the information received the external device available to the processing circuitry 56.

A person skilled in the art will now appreciate that further variations on the configurations shown in FIG. 1 through FIG. 6 are contemplated. Hereafter, portable monitoring device 50 will be discussed, but it is to be understood that all of the variations on portable monitoring device 50 (i.e. device 50, device 50a, device 50b . . . device 50f) can be applied to the following discussions according to the context of the following discussions.

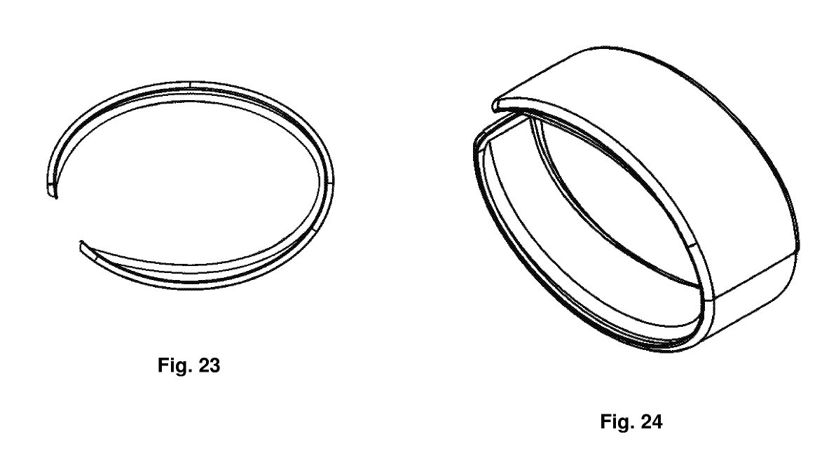

In certain embodiments, it is contemplated that at least a portion of the portable monitoring device 50 (including the one or more pulse profile sensor(s) 52 and/or physiological and/or environmental sensor(s) 54) is worn by the user, or affixed to the user, during operation wherein the housing of the device has a physical size and shape that facilitates coupling of the housing to the body of the user. For example, the portable monitoring device 50 can be a bracelet worn on an arm, wrist, ankle, waist, stomach, chest, leg, and/or foot, (and/or worn on finger as a ring, or worn on ear e.g. as ear-buds, headphones, earrings, glasses, headbands, hats, or other head gear, etc.). For example, the portable monitoring device 50 can be in a watch form worn on the wrist; for example it can be integrated with traditional watch functionality (such as indicating the time), moreover the traditional watch look can be maintained (such as have an analog and/or digital display or clock face); portable monitoring device 50 can also be (or integrated with) a smart watch. For example, existing (bracelet, watch, etc) designs can be modified e.g. in order to reduce development and/or manufacturing costs. It is presently preferred that the form factor of the portable monitoring device 50 allows performance of normal or typical activities without undue hindrance. The portable monitoring device 50 can include a mechanism (for example, a clip, strap, band and/or tie) for coupling or affixing the device to the body of the user. An example bracelet configuration is shown in FIG. 23 (side perspective view) and FIG. 24 (top perspective view). The components of device 50 (shown in FIGS. 1-6 and 22) can be contained within the bracelet housing shown in FIGS. 23 and 24. As a further example, the portable monitoring device 50 can be in a housing that clips on to an existing wearable device such as a watch, bracelet, headphones, glasses, article of clothing, etc. As a further example, the portable monitoring device 50 can be integrated into clothing such as undergarments (e.g. bra, undershirt, panties, briefs), tights, shirts, etc; portable monitoring device can also be a “patch” affixed to the body e.g. by the use of adhesives; for example, the “patch” form can have an advantage of being less susceptible to motion artifacts and/or an advantage of being flexible in terms of placement on the body. As a further example, the portable monitoring device can be implanted (partially or completely) into the body. As a further example, the portable monitoring device 50 (including the one or more sensors 52, 54) can be operated with minimal direct physical coupling to the body, or without direct physical coupling to the body (for example via using non-contact photoplethysmography, (SUN, Y et al., 2013)). In certain embodiments (for example, using MMSB sensor(s) (PHUA, C T, 2012), as pulse profile sensor(s) 52), portable monitoring device 50 can operate without direct contact and/or line of sight with the body.

During operation, the one or more pulse profile sensor(s) 52 and/or one or more physiological sensor(s) 54 generate data representing aspects of physiology that can be used (with the application of further processing via processing circuitry 56, if necessary) for predicting the desired nutrition-related metrics. During operation, the one or more environmental sensors 54 generate data (which may not directly correspond to physiology) that can be used (with the application of further processing via processing circuitry 56, if necessary) for predicting the desired nutrition-related metrics. For example, the environmental sensor 54 can be a motion sensor (e.g. accelerometer).

For example, the user's state (e.g. physiological, behavioural, environmental, etc.) may be dependent on ingestion of food, and portable device 50 can measure this state via sensors 52, 54 in order to predict (with the application of further processing via processing circuitry 56, if necessary) nutrition-related metrics. The user's state can include (but is not limited to) one or more of: cardiovascular (and/or hemodynamic) effects; metabolic effects; nervous system effects (e.g. central, peripheral, autonomic); gastric activity; hormonal effects; metabolite concentration changes, pH changes, body composition changes, and/or body mass changes. The user's state can include (but is not limited to) one or more of: activity (e.g. as detected by inertial sensors, muscle sensors, etc.); environment (e.g. as detected by location sensors, temperature sensors, sound sensors, light sensors, smell sensors (e.g. electronic nose), etc.). In addition, the user's state may have a time-dependence (non-limiting examples include: before ingestion (e.g. food preparation activities, anticipation of food); during ingestion (e.g. hand-to-mouth gestures, biting, chewing (“mastication”), and/or swallowing (“deglutition”)); and/or following ingestion (such as cleanup activities and/or effects of digestion)) which portable device 50 can measure via sensors 52, 54 (along with an appropriate time reference, e.g. a real-time clock); this time dependence can be used (with the application of further processing via processing circuitry 56, if necessary) to predict nutrition-related metrics. Though any physiological and/or environmental sensor(s) 54 in general can be used by device 50 to capture this state, pulse profile sensor(s) 52 have an advantage of being able to capture this state conveniently. For example, a pulse profile sensor 52 can capture this state (for example, but not limited to: the distribution of blood flow throughout the body as well as the cardiac timing from the heart) from a single (or a small number of) locations with respect to the body, and the choice of these one or more locations is flexible (e.g. virtually any blood vessel(s) can be used, including microvasculature; for example peripheral locations such as the ear, arm, and/or wrist can be used). The pulse profile sensor(s) 52 can be any sensors that measure a pulse profile, i.e. a cardiac-synchronized periodic waveform that reflects the pumping action of the heart and/or its effect on the vasculature e.g. the effects due to cardiac-induced blood flow. An exemplary pulse profile as generated by the pulse profile sensor(s) 52 is depicted in FIG. 8. For example, a pulse profile sensor 52 can be any sensor that measures vascular dimension (i.e. time-varying length, area, and/or volume) such as by plethysmography (e.g. photoplethysmography (PPG) (including non-contact PPG (SUN, Y et al., 2013)), impedance plethysmography (IPG) (WANG, J et al., 2011) (BUTTERFIELD, T, 2008) which has a potential advantage compared to PPG of requiring less power for achieving a given SNR, and/or Modulated Magnetic Signature of Blood (MMSB) (PHUA, C T, 2012) which has a potential advantage of being able to operate through material such as clothing). In other embodiments, a pulse profile sensor 52 can be any sensor that measures vascular pressure, preferably in a non-invasive manner, such as by sphygmography (e.g. with a piezoresistive or piezoelectric pulse sensor), applanation tonography, or by invasive methods such as by arterial (or venous) catheter. In further embodiments, a pulse profile sensor 52 can be any sensor that measures vascular flow, such as Doppler flowmetry, ultrasonic transit-time sensors, or electromagnetic flow meters.

For example, in a preferred embodiment, the pulse profile sensor(s) 52 are one or more photoplethysmography (PPG) sensors. Furthermore, the PPG sensor 52 can include associated amplification and/or processing circuitry (e.g. analog and/or digital processing circuitry) as a self-contained “sensor” component. For example, in one embodiment, the PPG sensor 52 has signal processing circuitry (e.g. “Analog Front End” (AFE), e.g. including photodiode amplification, ADC, LED control, and ambient light cancellation) implemented in an integrated circuit package, for example, the AFE4400 “Integrated Analog Front End for Heart Rate Monitors and Low Cost Pulse Oximeters” or the AFE4490 “Integrated Analog Front End for Pulse Oximeters” from Texas Instruments Incorporated. For example, the AFE4403 or the AFE4404 from Texas Instruments can be used. For example, the ADPD142 from Analog Devices, Inc. can be used (e.g. the ADPD142RG or the ADPD142RI). In one embodiment, multiple LED channels are used for the same LED in order to increase the effective sampling rate and potentially allow sample averaging (to cancel out noise) in order to increase SNR (e.g. the AFE4403 has two LED channels, they can both be used with a single LED in order to double the sample rate or reduce the noise amplitude by about √2 for the same effective sample rate). For example, the processing circuitry of PPG sensor(s) 52 can be optimized for low noise and/or low power; for example techniques, see (GLAROS, K N, 2011).

A variety of light sources and light detectors can be used for the PPG sensor 52. For example the PPG sensor 52 can consist of one or more Light Emitting Diodes (LEDs) and/or laser diodes and one or more photodiodes and/or phototransistors. The PPG sensor 52 can be selected to minimize the impact of noise (i.e. components of the signal that are not directly related to the blood volume changes of interest) or the performance of further processing stages by use of certain wavelengths of light. For example green light (wavelengths in the approximate range 500-565 nm, e.g. about 525 nm), red light (wavelengths in the approximate range 600-750 nm, e.g. about 660 nm) and infrared light (wavelengths in the approximate range 850-1000 nm, e.g. about 910 nm) are known to be favourable due to a high contrast with hemoglobin in blood. Shorter wavelengths such as blue and green (and/or closer spacing between light source(s) and light detector(s)) tend to measure vasculature nearer the surface while longer wavelengths such as red and infrared (and/or further spacing between light emitter(s) and light detector(s)) tend to measure deeper vasculature. An advantage of shorter wavelengths is that they can be less sensitive to certain kinds of motion artifact as well as have better optical contrast with plasma hemoglobin, however they can be more attenuated in amplitude by skin pigmentation (e.g. especially for darker skin); an advantage of longer wavelengths is that they can provide more a detailed pulse profile (e.g. less damped, e.g. less attenuation of higher harmonics) reflective of deeper blood circulation. In certain embodiments, an array of locations can be used to account for the fact that blood circulation can be more optimal in certain locations e.g. which are not necessarily known beforehand. Additionally, in certain embodiments, a combination of configurations (wavelength, source(s)/detector(s) spacing and/or geometry, sensor(s) 52 location, etc. together be simultaneously more informative for the prediction by processing circuitry of nutrition-related metrics than any single configuration. Indeed, in an embodiment any combination of wavelengths can be selected, for example based on signal quality, or combined, for example by an average signal of all the PPG sensors, or by calculating a separate set of features for each wavelength (“features” are further described below). As another example, the optical configuration, geometry, and/or mounting pressure can be selected by processing circuitry 56 e.g. based on demographics, and/or based on measurement of a quality metric (e.g. an SNR metric, e.g. as described below). As a further example, multiple sensors can be used in different locations with respect to the body (for example, the sensor(s) with the best signal quality can be selected, and/or each sensor can be used to calculate a separate set of features, used in combination for the regression model 206; “features” and “regression model” are further described below). For example, a correction can be applied by processing circuitry 56 to the acquired PPG signal to account for the potential changes due to sensor configurations; for example the correction can be the additive and/or multiplicative harmonic proportion spectrum correction as described below; for example the correct factor can be determined by laboratory measurements, and/or by measuring the different sensor configurations (e.g. simultaneously or otherwise close in time succession, in order to minimize the changes of other unrelated conditions) on the user and fitting the additive and/or multiplication factors to these measurements. In certain embodiments, a larger area light detector (e.g. larger area photodiode) can be used to maximize the acquired signal amplitude (in order to maximize SNR), for example multiple light detectors (e.g. photodiodes) can be used in parallel to acquire a total signal of greater total amplitude and/or greater total SNR. In certain embodiments, at least 2 light sources can be symmetrically placed around a light detector in interfacing with the user's tissue; alternatively, at least 2 light detectors can be symmetrically placed around a light source in interfacing with the user's tissue; a potential advantage these configurations is the minimizing of certain kinds motion artifact. Further means for minimizing the impact of noise will be described below.

In certain embodiments, PPG sensor(s) 52 and processing circuitry 56 can be configured in order to implement one or more aspects of Masimo “Signal Extraction Technology®” (GOLDMAN, J M et al., 2000) (GRAYBEAL, J M et al., 2004), for example Discrete Saturation Transform® (DST), FST®, SST™, MST™, and/or Low Noise Optical Probe™ (LNOP) sensor design. For example, Masimo DST can be used to suppress the effect of venous blood on the PPG signal, minimizing the effect of certain kinds of noise such as motion artifact which venous blood is more susceptible to. For example, the techniques of U.S. Pat. No. 5,632,272, entitled “Signal processing apparatus”, Mohamed K. Diab et al, the contents of which are incorporated herein by reference, can be applied by sensor(s) 52 and/or processing circuitry 56. For example, the techniques of U.S. Pat. No. 5,769,785, entitled “Signal processing apparatus and method”, Mohamed Kheir Diab et al, the contents of which are incorporated herein by reference, can be applied by sensor(s) 52 and/or processing circuitry 56. For example, in certain embodiments, sensor(s) 52 and/or processing circuitry 56 can be configured to apply the Minimum Correlation Discrete Saturation Transform (MCDST) technique e.g. in order to minimize the effects of noise such as motion artifacts (YAN, Y S et al., 2008).

The one or more motion sensor(s) 54 of the embodiments of the present specification can refer to any one or more inertial sensors e.g. accelerometer (e.g. 1, 2, 3 axis), gyroscope, magnetometer, compass, GPS; for example, multiple sensors can be provided in the same package, for example with the option of fusing the motion information e.g. a 6-axis motion sensor integrating 3-axis accelerometer and 3-axis gyroscope; for example, an inertial measurement unit (IMU) can be used.

The PPG sensor 52 (or any sensor 52, 54) can output discrete samples for further processing by the processing circuitry 56. In this case, the sampling rate can be pre-set, for example at about 100 samples/sec, or variable, for example in order to find a trade-off between signal quality and power consumption. Furthermore, the sampling can happen with uneven spacing. For example, the sampling timing can happen in a manner (for example, a pseudo-random sampling pattern) that supports the use of compressive sensing algorithms in order to reduce power consumption of the PPG sensor 52 (e.g. by reducing the effective sampling rate), while still achieving a required signal quality. The use of compressive sensing will be further described below. Furthermore, the “sampling” can be continuous (for all or part of the sensors 52, 54 and/or processing circuitry 56), for example in the case of analog signal processing.

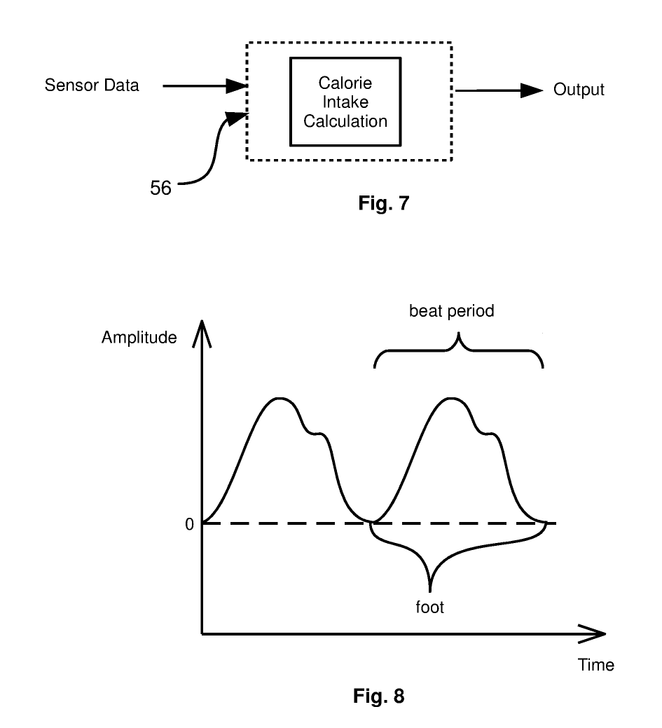

As shown in FIG. 7, the processing circuitry 56, using (i) data which is representative of aspects of the user's pulse profile; and/or (ii) data which is representative of other physiological and/or environmental factors; calculates energy and/or caloric intake of the user.

The processing circuitry 56 (as well as any other processing circuitry, such as processing circuitry contained within pulse profile sensor 52) can be discrete or integrated circuits, and/or one or more hardware-implemented state machines, processors (e.g. one or more central processing units (CPUs), suitably programmed, e.g. by executing computer-readable instructions implementing a state machine) and/or field-programmable gate arrays (FPGAs) (or combinations thereof); indeed any circuitry now known or later developed can be employed to calculate the energy and/or caloric intake of the user based on sensor data. As further examples, processing circuitry 56 (as well as any other processing circuitry) can be analog circuitry, optical circuitry, mechanical circuitry, quantum circuitry, and/or some hybrid. In operation, the processing circuitry 56 can perform or execute one or more applications, routines, programs and/or data structures that implement particular methods, techniques, tasks or operations described and/or illustrated herein. The functionality of the applications, routines, or programs can be combined or distributed. Further, the applications, routines or programs can be implemented by the processing circuitry 56 using any programming language whether now known or later developed, including, for example, assembly, FORTRAN, C, C++, BASIC, Java, Python, and MATLAB, whether compiled or uncompiled code; all of which are intended to fall within the scope of the present specification.

Explained in greater detail, the processing circuitry 56 can be configured to calculate other nutrition-related metrics besides caloric intake. Other nutrition-related metrics can include for example: (a) calories categorized into the macronutrient type (for example, carbohydrates, proteins, and fats) (for example, by absolute calories and/or by caloric proportion; for example, a meal of 550 kcal, can be expressed as having 150 calories of carbohydrates, 100 calories of proteins, 300 calories of fats and/or can be expressed in caloric proportions as 27% from carbohydrates, 18% from proteins, 55% from fats); (b) the equivalent mass and/or volume for a macronutrient type (for example, mass of carbohydrates, mass of proteins, and mass of fats); (c) a further breakdown (e.g. by caloric proportion, mass, volume, mass proportion, volume proportion, etc.) for carbohydrates (for example, starches, sugars; or bread-like starches, pasta-like starches, glucose-like sugars, fructose-like sugars; and/or mass of fibre intake); (d) a further breakdown (e.g. by caloric proportion, mass, volume, etc.) for proteins (for example, animal-based proteins, plant-based proteins); (e) a further breakdown (e.g. by caloric proportion, mass, volume, etc.) for fats (for example, saturated fats, unsaturated fats; or in terms of omega-3/omega-6 ratio); (f) the glycemic index; (g) the glycemic load; (h) state of hydration (e.g. absolute overhydration (“OH”) in units of volume, or relative overhydration (“rel. OH”), further described below); Furthermore, nutrition-related metrics corresponding to intake of micronutrients can be calculated, such as vitamins, minerals, fibre, and/or phytonutrients, including: (i) sodium intake (e.g in units of mg or mmol); (j) potassium intake (e.g. in units of mg or mmol); (k) potassium/sodium intake ratio (e.g. “K+/Na+”, in units of mmol/mmol); (1) caffeine intake (e.g. in units of mg, or in units of mg/kg body weight); (m) alcohol intake (e.g. in units of mass, volume, or “standard drinks” (e.g. where a “drink” is about 14 g of alcohol); or, in terms of blood alcohol concentration); (n) phytonutrient intake, e.g. in terms of a phytonutrient index [%] (MCCARTY, M F, 2004), and/or in terms of change in antioxidant capacity (e.g. change in total plasma antioxidant capacity; in units of umol, or in % change; total antioxidant capacity can also be reported, e.g. in units of uM or in units of nmol/mg of protein) (GHISELLI, A et al., 2000). The examples in the preceding sentence can be broken into categories pertaining to a given window of time (for example, the past day, or a given week) or categories pertaining to each distinct meal, for example. Means for calculating these metrics will be discussed in greater detail below.

For example, in combination or in lieu of caloric intake, the total mass or volume of food intake can be calculated by processing circuitry 56. It should also be noted that caloric intake as measured in kcal is in certain parts of the world (such as North America) commonly expressed as “Calories”; also the units of kJ can be used with the appropriate conversion e.g. 1 kcal=4.184 kJ.

For example nutrition-related metrics (such as those mentioned above) can be predicted and/or expressed by portable device 50 in terms of proportion of recommended daily intake (e.g. where recommended daily intake can be for the general population, or adjusted to the user based on demographics and/or personal goals). For example, if the user's daily caloric target is 2000 kcal, caloric intake for a given meal (or period of time) of 500 kcal can be expressed as 25% of recommended caloric intake.

In certain embodiments, where caloric intake is predicted by processing circuitry 56, the caloric intake can be the Metabolisable Energy [kcal] (as is available as the “Calories” directly from the Nutrition Facts panels at present in North America). In certain other embodiments, the caloric intake can be the ME with unavailable carbohydrates (i.e. “dietary fibre”) specifically accounted for, for example according to the equation: ME [kcal]=3.75*AC+2*DF+9*F+4*P+7*Alc, where: AC is mass of available carbohydrates (or monosaccharide) [g], DF is mass of dietary fibre (or “unavailable carbohydrates”), F is mass of fats [g], P is mass of proteins [g], and Alc is mass of alcohol [g] (e.g. see FIG. 3 and Table 1 of (LIVESEY, G, 2001)); for example, the variables AC, DF, F, P, Alc can be acquired during the training process (further described below) for a given meal from the Nutrition Facts labels. An advantage of the embodiments of the previous sentence is that the energy intake due to fibre is better accounted for (e.g. especially in low caloric dense, high-fibre foods). In general, the ME values (labelled as “Calories”) from the Nutrition Facts labels tend to overestimate the energy intake available to the body (e.g. in the ME sense) from certain foods by not always accounting for fibre, especially low caloric density and/or high-fibre foods.

In certain other embodiments, the caloric intake can be the Net Metabolisable Energy (NME) (see FIG. 3 and Table 1 of (LIVESEY, G, 2001), for example according to the equation: NME [kcal]=3.75*AC+1.5*DF+9*F+3.2*P+6.3*Alc, where: AC is mass of available carbohydrates (or monosaccharide) [g], DF is mass of dietary fibre (or “unavailable carbohydrates”), F is mass of fats [g], P is mass of proteins [g], and Alc is mass of alcohol [g] (e.g. see FIG. 3 of (LIVESEY, G, 2001)); for example, the variables AC, DF, F, P, Alc can be acquired during the training process (further described below) for a given meal from the Nutrition Facts labels. An advantage of the embodiments of the previous sentence is that the energy intake components due to fibre, protein, and alcohol are better accounted for (e.g. especially in low caloric dense, high-fibre foods and/or high protein foods and/or high alcohol foods). In general, the ME values from the Nutrition Facts labels (under “Calories”) tend to overestimate the energy intake available to the body (e.g. in the NME sense) from certain foods, especially low caloric density (and/or high fibre) foods, high-protein foods, and/or alcohol.

In certain embodiments the processing circuitry 56 can be configured to calculate other nutrition-related metrics; indeed any metric related to the intake of food and/or any substance in general and/or its effects on the user can be used as metrics. For example, the duration, rate and/or time distribution of ingestion (e.g. the time spent biting, chewing, and/or swallowing; e.g. the rate of ingestion expressed as kcal/minute; e.g. whether or not majority of ingestion for a meal took place within about 20 minutes) can be calculated. For example, appetite and/or satiety (e.g. before a meal, after a meal) can be calculated. For example, the state of being bloated and/or constipated can be calculated. For example, metrics describing and/or quantifying the elimination of feces and/or urine can be predicted. For example, metrics describing and/or quantifying the intake of drugs and/or medications (e.g. recreational, prescription, etc.) can be predicted. In certain embodiments, in combination with or in lieu of nutrition-related metrics, any health-related metrics can be predicted, for example: metrics reflecting the state of health and/or wellness; metrics reflecting the state of exercise, stress, and/or sleep; metrics reflecting the diagnosis and/or progress of a condition, illness, infection and/or disease, etc. In certain embodiments, in combination with or in lieu of nutrition-related metrics and/or health-related metrics, any metrics in general can be predicted e.g. specific activities and/or behaviours of the user, effects of the environment on the user, etc.

Processing Chain

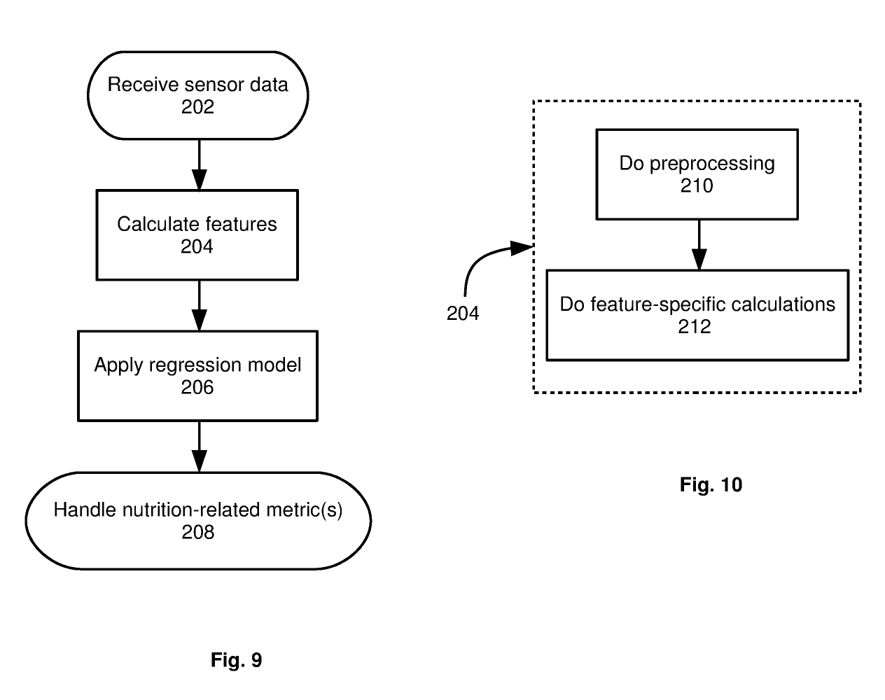

In the preferred embodiment, the processing circuitry 56 is configured to implement a process (hereafter referred to as the “processing chain”) based on the flowchart of FIG. 9. In the processing chain, sensor data is received by processing circuitry 56 (e.g. from the sensors 52, 54, and/or from memory connected to or integrated with processing circuitry 56 used to store sensor data at least temporarily) (block 202), one or more feature(s) are calculated by processing circuitry 56 from the received sensor data (block 204), and the regression model (also known in the art as a “stochastic estimator”) is applied by processing circuitry 56 to calculate (or “predict”) nutrition-related metrics from features (block 206); the resulting nutrition-related metrics can then be handled (block 208), for example by displaying to the user via user interface 58 and/or output to an external device. The regression model 206 is the result of a training process where a machine learning algorithm is used to find a model for calculating nutrition-related metrics from features, given examples of corresponding (features measurement, nutrition-related metric measurement) pairs. The (features measurement, nutrition-related measurement) pairs correspond to (inputs, output) in the machine learning terminology, or equivalently (inputs, target) or (inputs, label). Hereafter, each (features measurement, nutrition-related metric measurement) pair is referred to as a datapoint, and a collection of datapoints is referred to as a dataset. In certain embodiments, each datapoint corresponds to a single meal, while in other embodiments, each datapoint corresponds to a set window of time. The regression model 206, machine learning algorithm, and training process are described in greater detail below.

It should be noted that in certain aspects of certain embodiments, referring to FIG. 9, the calculation of features (block 204) by processing circuitry 56 may be minimal or not required, that is, the regression model 206 makes predictions from raw or minimally processed sensor data. Additionally, in certain embodiments, a regression model 206 may not be required (for some or all of the calculations of nutrition-related metrics), and instead the calculations in block 204 are sufficient to calculate the certain nutrition-related metrics (e.g. caloric or macronutrient intake), as will be further described below.

Features

Referring now to FIG. 10, in an embodiment, one or more feature(s) can be calculated from sensor data by processing circuitry 56 via a preprocessing step (block 210) followed by a feature-specific calculations step (block 212).

It should be noted that each particular feature (or set of features) calculated by processing circuitry 56 at block 212 can dictate the required pre-processing to be performed by processing circuitry 56 at block 210, and that different features representing data from more than one sensor 52, 54 can be used. If different features to be calculated at block 212 by processing circuitry 56 share at least some sensor data and/or preprocessing steps in common, it is reasonable to share the corresponding intermediate calculations between them in order to reduce computational requirements. Processing circuitry 56 can therefore be configured, when two or more features at block 212 require the same preprocessing activities, to perform those preprocessing activities only once, and employ the results for each feature whose calculation requires those results at block 212.

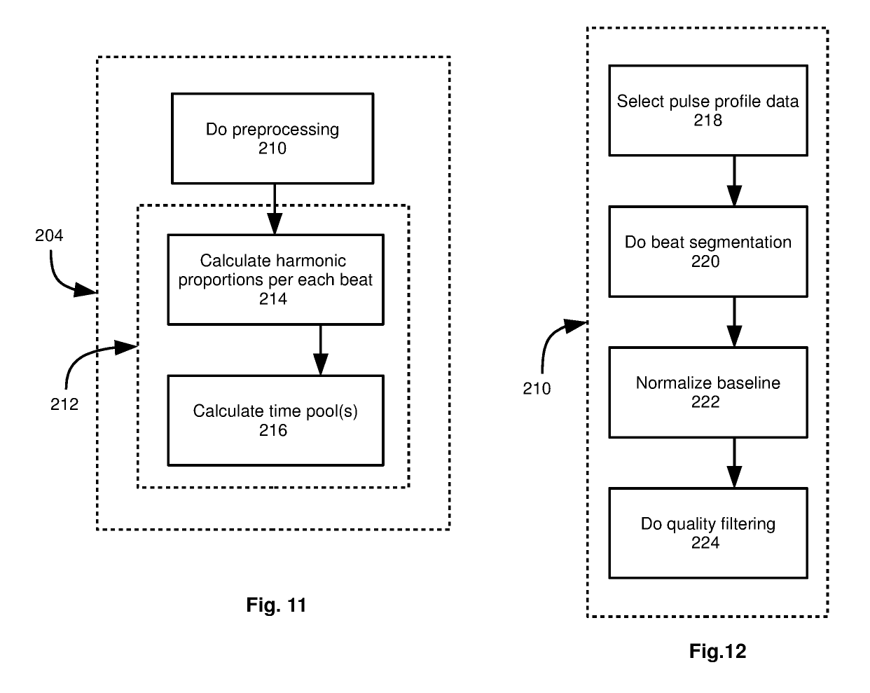

Referring now to FIG. 11, in the preferred embodiment, time-pooled harmonic features can be calculated by processing circuitry 56 from data received at processing circuitry 56 from a pulse profile sensor 52. It is also understood that in certain embodiments, the order of processing steps in FIG. 11 can be different than that shown. For example, in an embodiment, the pre-processing (block 210 of FIG. 11) contains a quality filtering step (block 224 of FIG. 12) that requires use of harmonic features (calculated in block 214 of FIG. 11, as will be described below); thus this part (block 224) of the pre-processing 210 can be performed by processing circuitry 56 after the calculate harmonic features step (block 214) in order to avoid repeating redundant calculations in the interests of reducing computation requirements.

The pre-processing step can be implemented according to the process shown in FIG. 12. In Select pulse profile data (block 218) a window of pulse profile data (e.g. previously stored-in-memory) can be selected by processing circuitry 56 for further processing. For example, the window for further processing can be a pre-set window (e.g. preconfigured in processing circuitry 56) up to the present time e.g. the last about 3 hours or the last about 5 minutes, or as in the preferred embodiment, a window referenced to the start time of the last meal, e.g. starting from about 30 minutes before the last meal start time and ending about 4.5 hours after the last meal start time. (For example, the meal start can be defined as the time that the user starts ingestion of food, e.g. “the first bite” of a given meal.) The means for determination of the last meal start time will be further described below.

In certain embodiments, up-sampling and interpolation (e.g. 2X up-sampling or 4X up-sampling) of the pulse profile data can be performed by processing circuitry 56 prior to further processing in order to improve the time resolution of the processing steps that follow (SUN, Y et al., 2013). For example, up-sampling and interpolation can be performed by processing circuitry 56 prior to beat segmentation (block 220), or immediately prior to other steps such as the Fourier transform step (block 226).

In beat segmentation (block 220 of FIG. 12), the start time of each beat can be calculated by processing circuitry 56. In one embodiment, the start of each beat is defined as the local minimum point of the given cycle (the “foot”), as shown in FIG. 8. However, it is understood by one skilled in the art that other points on the waveform cycle can be used (for example, the “rising edge”, e.g. calculated by processing circuitry 56 as the maximum of the first derivative (or first finite difference) of the given cycle). Numerous beat detection algorithms can be used by processing circuitry 56 for calculating the start of each beat, including those available in the prior art. A key criterion for the beat detection algorithm executed by processing circuitry 56 is how it handles ambiguous beats in the presence of noise in the pulse profile signal. In the preferred embodiment, processing circuitry 56 is configured at block 220 to be liberal in selecting beats, even when the beats are ambiguous due to noise. In this embodiment, erroneous “beats” that are mistakenly selected by processing circuitry 56 at block 220 can be detected and rejected or corrected by later processing stages, e.g. a quality filter, as will be described below. However, it is understood that other strategies for detecting valid beats in the presence of noise can be employed, and fall under the scope of the present specification.

In the normalize baseline step (block 222 of FIG. 12), the baseline component (sometimes known in the art as the “DC component”) of the pulse profile signal can be removed or suppressed by processing circuitry 56 in order to aid further processing. In one embodiment, this step is performed by the use of a linear high-pass filter. For example, an Infinite Impulse Response (IIR) or Finite Impulse Response (FIR) filter can be used by processing circuitry 56, for example with a 2^{nd} order high-pass cut-off frequency of about 0.5 Hz. In another embodiment, the normalize baseline step (block 222 of FIG. 12) is performed by processing circuitry 56 for each beat by interpolating a line segment through the foot immediately preceding the beat and the foot immediately following the beat, and then subtracting the line segment samples from the beat samples. Furthermore, the two methods described in this paragraph for suppressing the baseline are complementary and may be combined; it is also understood that any method for removing or suppressing the baseline of the pulse profile signal can be used and fall under the scope of the present specification.

In the apply quality filtering step (block 224 of FIG. 12), noise in the pulse profile signal (which is any component of the pulse profile signal, or of a partially processed pulse profile signal that is not related to the underlying cardiac-induced pulsations) is handled by processing circuitry 56 in order to reduce the effect of noise on the accuracy/reliability of further stages of processing. For example, it is well known in the art that motion artifacts tend to constitute a significant portion of noise in the pulse profile signal (in particular, when the pulse profile signal is a PPG signal). Other possible sources of noise include poor blood circulation to tissue local to the sensor(s) 52, and noise from electronic processing circuitry (whether in sensor(s) 52 or in processing circuitry 56 itself). Additionally, data could be corrupted or missing due to the user removing the device 50 or sensor(s) 52 (or otherwise wearing device 50 or sensor(s) 52 sub-optimally, in the case of a wearable device 50), or neglecting to maintain power to the device 50 (e.g. recharge the batteries), for example.

There are two example strategies that can be employed by processing circuitry 56 in quality filtering (block 224) to reduce the effect of noise on the accuracy/reliability: a) rejection of excessively noisy portions of pulse profile signal i.e. not using these noisy portions of data in further stages of processing; and b) suppression of noise in order to recover the desired waveform corresponding to the cardiac-induced pulsations in the pulse profile signal. Additionally, these strategies are complementary and can be combined in certain embodiments. In one embodiment, excessively noisy portions of pulse profile signal are rejected by processing circuitry 56 on a per-beat basis, by evaluating beat period statistics (further described below), beat morphology statistics (such as beat harmonic proportions, further described below) and/or rejected in the presence of motion above a certain threshold as detected by a motion sensor 54. In one embodiment, low pass filters (e.g. analog and/or digital) can be used to suppress excess noise energy outside of the signal band (for example, if harmonic #7 is the highest frequency of interest, and the highest heart rate of interest is about 100 BPM, a low-pass filter with a cut-off frequency of about 100/60*7=11.7 Hz or higher can be effective); additionally the cut-off frequency can be adaptive e.g. as the heart-rate changes. In one embodiment, excessive noise in the pulse profile signal can be suppressed by processing circuitry 56 according to the method described in (REDDY, K A et al., 2009). In order to utilize this method, the beat detection step (block 220) should be configured to be conservative in detecting beats, so that any beats that it selects are highly likely to have the correct beat start timing (and ambiguous beats are rejected). As an additional example, compressive sensing techniques can be used by processing circuitry 56 for de-noising of the pulse profile. In certain embodiments, one or more methods from the prior art can be employed in order to suppress noise in the pulse profile signal, e.g. for motion artifacts and/or poor mechanical and/or optical coupling with the desired cardiac-induced blood pulsations and/or excessive electrical noise. For a non-exhaustive review of exemplary techniques, see (TAMURA, T et al., 2014).

In one embodiment, each beat can filtered based on successive period ratios: a beat is rejected by processing circuitry 56 if the ratio between the beat's period and the preceding beat's period is excessively deviated from unity. For example, if the ratio is greater than about 1.2, or is less than about 0.8, the beat can be rejected. Similarly, the ratio can be between the beat period and the following beat's period. Furthermore, both ratios can be calculated by processing circuitry 56, and the beat can be rejected if either ratio is excessively deviated from unity. In an embodiment, processing circuitry 56 can filter the beats by period according to percentiles of the beat periods in a window: a beat can be rejected if the beat's period is excessively deviated from the median period, where the median is calculated over a moving window, for example over the last about 1 minute of beats. For example, a beat can be rejected by processing circuitry 56 if the beat period is less than about Q1−1.5*IQR or greater than about Q3+1.5*IQR, where Q1 is the 1^{st} quartile (or 25^{th} percentile), Q3 is the 3^{rd} quartile (or 75^{th} percentile) and IQR is the Interquartile Range (that is, the difference between Q3 and Q1). In an embodiment, the beat filtering is the combination of the methods disclosed in this paragraph: filtering on successive period ratios, followed by filtering on period according to percentiles.

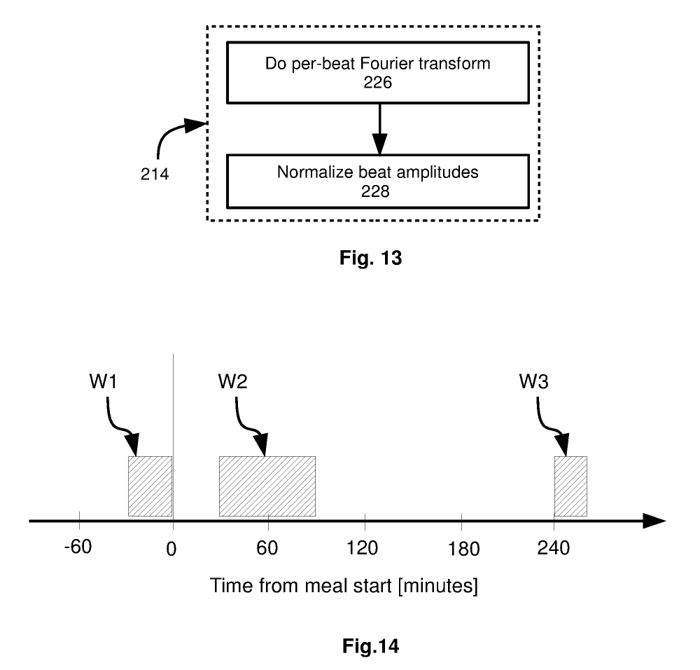

In another embodiment, either in combination or in lieu of the disclosed methods based on beat period filtering, beats may be rejected by processing circuitry 56 based on the harmonic proportions. Techniques applied by processing circuitry 56 for calculating harmonic proportions are described below (with reference to block 214 of FIG. 11 and block 226 of FIG. 13). In this embodiment, for each beat, each harmonic proportion (e.g. harmonic proportion 1, harmonic proportion 2, etc.) can be compared against the percentile of the harmonic proportions of previous beats, in a manner similar to that described in the previous paragraph, but with harmonic proportions substituted for the beat period. Besides harmonic proportions, the harmonic phases can also be used by processing circuitry 56 for rejecting beats, e.g. in a similar manner (i.e. by comparing to percentile phases determined from a moving window). In addition, the harmonic proportions and/or phases can be compared against typical values (e.g. stored in a memory integrated with or otherwise connected to processing circuitry 56) for the general population (or any user population of interest) in determining which beats should be rejected. Typical values for the general population can be found by obtaining the sample mean of each variable for a set of users. The precise values will depend on the particular type and configuration of pulse profile sensor 52 used, but example values are available in the first table of U.S. Pat. No. 5,730,138, entitled “Method and apparatus for diagnosing and monitoring the circulation of blood”, Wang, W K, (column 6, line 55 to column 7, line 20), incorporated herein by reference. In one embodiment, the coefficient of variance (=standard deviation/mean) can be calculated by processing circuitry 56 for each harmonic proportion and/or harmonic phase for a window of beats (e.g. a moving window). In this embodiment, the window of beats (e.g. an about 60 seconds window) can be accepted if the coefficient of variance is less than acceptable limits, for example, about 7% for harmonic proportions 1-4, and about 15% for harmonic proportions 5 or greater. Furthermore, any other means for rejecting a detected beat based on the deviation of the pulse profile (e.g. “waveform” or “morphology”) from the expected shape (or any parameters that quantify waveform shape or morphology) can be used, and fall under the scope of the present specification. Furthermore, in the techniques of this paragraph where harmonic proportions are used, the complex harmonic proportions can be used; for example processing circuitry 56 can filter the beats by period according to percentiles of the complex harmonic proportions in a window: a beat can be rejected if the beat's complex harmonic proportion is excessively deviated from the median complex harmonic proportion, where the median is calculated over a moving window, for example over the last about 1 minute of beats.

Other mechanisms, either in lieu of or in combination with the quality filtering step (block 224 of FIG. 12) are also contemplated for minimizing the impact of noise on the pulse profile signal and/or on the functioning of device 50 in determining nutrition-related metrics and providing such nutrition-related metrics to the user. As described above, the pulse profile sensor 52 can be selected and configured in order to minimize the impact of noise. In other embodiments, the noise can be handled (that is, removed partially or completely) by processing circuitry 56 in later stages of processing. For example, beat segmentation can be optimized for being more robust to noise. For example, the regression model 206 trained by machine learning (described below) can in certain circumstances perform with better prediction performance if noise is left in the signal, by incorporating the noise into its predictive model. The main means for determining if one configuration (e.g. of regression model 206, and/or of any other component of the complete processing chain) is better than the other is by evaluating predictions from the complete processing chain on a validation dataset, as described below, under the training process. In an embodiment, if there is insufficient valid data to use a full regression model 206, a reduced regression model 206 can be used (for example in the case described below where time-pooled features are used, the threshold for sufficient valid data in a given window (e.g. W1, W2, or W3, with reference to FIG. 14) could be about 10% of expected data (e.g. expected beats=median heart rate [bpm]*window length [min]), and a model trained for only the subset of time-pooling windows with sufficient data used to make a prediction). In a further example, if there is insufficient data to make a prediction, the user interface 58 can indicate that a meal occurred but there was insufficient data for a prediction, and/or that there was uncertainty whether or not a meal occurred for a given window of time.

Referring now to FIG. 11, in the preferred embodiment, the Calculate harmonic proportions step (block 214) can be implemented by processing circuitry 56 as a process represented by the flowchart of FIG. 13, where first a Fourier transform is calculated by processing circuitry 56 for each beat (block 226), and second a normalization by amplitude is applied by processing circuitry 56 to each beat (block 228). In the preferred embodiment, the Fourier transform 226 is applied to each beat by processing circuitry 56, where the pulse profile can be decomposed according to the relationship expressed in the following equation:

$$P ( t ) = {a}_{0} + {\sum }_{n = 1}^{N} {a}_{n} cos ( \frac{2 π n}{T} t + {ϕ}_{n} ) , \qquad ( 1 )$$

Where: P(t) is the pulse profile signal (e.g. applied to a single beat), t is the time index (e.g. discrete or continuous), n is the Fourier or harmonic index, N is the maximum harmonic retained, aₙ is the n^{th} harmonic amplitude, T is the period of a given beat, and φₙ, is the n^{th} harmonic phase (e.g. referred to the beat start (e.g. foot, or edge, etc.), or to the 1^{st} harmonic phase).

In the preferred embodiment, processing circuitry 56 can apply the Fourier transform 226 according to Equation 1, where P(t) is a segment of the pulse profile over a single beat, and the start point of the beat is set to the same point on the beat cycle (e.g. foot, or edge, etc) when the Fourier transform 226 is applied to a given beat. In other embodiments, P(t) can be a segment of the pulse profile over multiple beats, e.g. an integer multiple of beats (e.g. 20 beats, where the segment's start and end points are aligned to the same point on the beat cycle e.g. foot, or edge, etc.) or multiple beats that aren't necessarily an integer multiple of beats (e.g. all the beats within a given 20 second segment) where the segment's start and end points are not necessarily aligned to point(s) on the beat cycle.

The choice of the maximum harmonic retained, or N in Equation 1, is pre-configured in processing circuitry 56, and is dependent on the particular implementation: a larger value of N can have more information about the desired nutrition metric(s), but can also contain more noise (as the amplitude of aₙ decreases as n increases). For example, N=7 can be used. Techniques for optimizing the noise performance (along with other competing performance factors such as power) are described below; we have found that retaining more harmonics that are not dominated by noise results in lower error for prediction nutrition-related metrics.

In the preferred embodiment, the Fourier transform 226 is implemented by a Fast Fourier Transform (FFT) algorithm, for example, a mixed-radix FFT. It is understood that the Fourier transform can be real-valued, or complex-valued with the appropriate conversion into amplitude and/or phase (“polar coordinates”) as necessary. In certain embodiments, where not all Fourier coefficients are used, the Fourier transform can be simplified by only calculating the Fourier coefficients that will be used.

With reference to block 228 of FIG. 13, the harmonic amplitudes (a₁, a₂, . . . ) can be amplitude normalized by dividing by a₀ (or equivalently, by dividing by the mean value of the pulse profile signal for the beat) in order to obtain the harmonic proportions, according to the following equation:

hpₙ=aₙ/a₀, (2)

Where: hpₙ is the n^{th} harmonic proportion, aₙ is the n^{th} harmonic amplitude, a₀ is the 0^{th} harmonic amplitude (equivalent to the mean amplitude of P(t) for the beat period), and 1<n<N. For the average amplitude a₀ in Equation 2, the amplitude of P(t) is referenced to the minimum (“foot”) for each given beat, as shown in FIG. 8.

In another embodiment, the denominator of Equation 2 can be replaced by another quantity, for example the Root Mean Square (RMS) of harmonic amplitudes, where harmonics 1 or higher are used (or the time-domain RMS amplitude of the beat after subtracting the mean value), the total power after subtracting the mean, or the peak-peak amplitude of a given beat.

Referring now to block 216 of FIG. 11, the time pooling of the harmonic features (or any features in general, where the particular feature is substituted for “harmonic proportion” in the discussions herein) can be implemented by processing circuitry 56 for a given window of time by calculating the arithmetic mean of each harmonic proportion for all valid beats in said window of time. For example, the relationship expressed in Equation 3 can be used:

$$\overline{{hp}_{n , W ι}} = \frac{1}{L} {\sum }_{Wi}^{ } {hp}_{n} [ k ] , \qquad ( 3 )$$

Where: h̅p̅_̅{̅n̅,̅W̅i̅}̅ is the time pooled n^{th} harmonic proportion for time window Wi, L is the number of valid beats in time window Wi, Z_{Wi}(.) is the sum over all valid beats in time window Wi, hpₙ[k] is the n^{th} harmonic proportion for the beat at time k, and k is the beat index (or in general, time index).

For example, with reference to FIG. 14, in the presently preferred embodiment three time windows are used: i) W1: from about 30 minutes before the meal start up to about the meal start, ii) W2: from about 30 minutes after the meal start to about 90 minutes after the meal start, iii) W3: from about 240 minutes after the meal start to about 260 minutes after the meal start. For each of these windows, the mean harmonic proportion is calculated by processing circuitry 56, for harmonics 1, 2, 3, 4, 5, 6, and 7, though other harmonics can also be applied. Furthermore, average (i.e. mean) phase can be calculated for a given window. For mean phase, the arithmetic mean of the complex Fourier coefficients (e.g. “vector averaging”, also described below) can be calculated by processing circuitry 56, and then the resulting mean complex coefficient can be converted into the mean phase value (real-valued; e.g. in units of radians or degrees). For example, in the preferred embodiment, phases 4, 5, 6 are averaged for the W2 time window described in FIG. 14 (from about 30 minutes after the meal start to about 90 minutes after the meal start) and used as features for regression model 206. It will now be apparent to one skilled in the art that other windows besides the three specified above can be used, and it can also be advantageous for the windows to overlap, and other pooling operations besides the arithmetic mean can be used (e.g. median, or maximum) as will be described below with reference to “Convolutional Neural Networks”. To summarize, in the preferred embodiment, 24=7*3+3*1 time-pooled harmonic features are calculated for use in regression model 206, corresponding to 7 harmonic proportions averaged over 3 time windows and 3 harmonic phases averaged over 1 time window.

In certain embodiments, the pooling operation for time-pooling of features by processing circuitry 56 can be an operation other than an arithmetic mean; for example an RMS averaging operation can be used, or a median operation, or a max operation. In certain embodiments, the pooling operation can be performed on the complex harmonic proportions (e.g. complex harmonic coefficients calculated by a Fourier transform according the relationship expressed in Equation 1, optionally with amplitude normalization applied as in Equation 3). In these embodiments, the pooling operation of Equation 3 can be an arithmetic mean applied to the complex harmonic proportions (e.g. such an averaging is known in the Electrical Engineering art as “vector averaging”; a potential advantage over an average (such as arithmetic mean or RMS averaging) performed on the amplitudes and/or phases (i.e. averaging of the polar co-ordinates directly) is that signal (typically noise such as motion noise or circuit noise) that is uncorrelated in phase with the beats is typically cancelled by the averaging. The resulting time-pooled complex harmonic proportions can be converted to amplitude and/or phase (“polar coordinates”) e.g. to be used as features in regression model 206, or used directly in a regression model 206 which accepts complex Fourier coefficients (described further below with reference to “complex regression model”).

Determining Meal Start Time

In one embodiment, the meal start time can be manually specified by the user, for example by a button (e.g. a single press indicates the start of a meal) in user interface 58 of the portable monitoring device 50, and/or by an application on an external device (such as a mobile phone). In this embodiment, the entry of meal start time can happen at about the moment of meal start, or can happen at another time (for example, in anticipation and/or planning ahead of a meal, or in retrospect).

In an embodiment, the occurrence of meals and the meal start times can be automatically detected by processing circuitry 56 from one or more physiological and/or non-physiological sensor(s) 54 (and/or the pulse profile sensor 52), and their respective features or parameters. For example, the Heart Rate Variability (HRV) parameters (such as LF/HF ratio, or SDNN/RMSSD ratio, described in further detail below, with respect to feature and sensor variations) can be compared to a pre-set threshold, where the meal start is calculated as the point at which the threshold (determined and pre-configured in processing circuitry 56 by hand-tuning against a training set, for example) is exceeded. Low-pass filtering (e.g. a moving average filter with window of about 1 minute) and/or hysteresis (e.g. requiring the threshold to be exceed for at least a given contiguous period of time, e.g. about 20 minutes) can be applied by processing circuitry 56 to improve reliability of automatic meal start detection (e.g. reduce false positives and/or false negatives). Exemplary techniques for detecting meal start given features or parameters determined from one or more sensors, specifically via calculating HRV parameters, are available in U.S. Pat. No. 8,696,616, entitled “Obesity therapy and heart rate variability”, Tamara, C, et al., in particular the text from column 16 line 12 through to column 18 line 18, which are incorporated herein by reference.

In certain embodiments, Activity Detection techniques (for example based on the accelerometer sensor 54) can be used to determine whether a meal has occurred as well as the meal start time (DONG, Y, 2012) (LAGUNA, J O et al., 2011). For example, hand gestures associated with food ingestion (such as hand-to-mouth gestures) can be detected. Additionally, biting, chewing, and/or swallowing can be detected by processing circuitry via appropriate sensor(s) 54 (e.g. inertial sensors); aside from detecting the occurrence and/or start time of a meal these variables can also be used to in estimating nutrition-related metrics (e.g. caloric intake) for a meal, e.g. by providing features in regression model 206 and/or by direct calculations without a regression model 206 (AMFT, O et al., 2008) (PAULO, L M, 2010). For example, processing circuitry 56, appropriately configured, can detect the occurrence of a swallow via a respiration sensor 54 (DONG, B et al., 2014). As a further example, the respiration signal can be acquired from a pulse profile sensor 52 via application in processing circuitry 56 of one or more appropriate signal processing techniques (MEREDITH, D J et al., 2012).

As another example, food preparation activities (such as cooking (e.g. microwaving), unpackaging, setting a table, etc.) typically precede the ingestion of food; additionally, clean-up activities can follow the ingestion of food and can be detected with appropriate sensor(s) 54 (e.g. inertial sensors).

In an embodiment, the occurrence of meals and the meal start times can be automatically detected by processing circuitry 56, by applying the sliding window method (FORSYTH, D A et al., 2011). In such an embodiment, processing circuitry 56 can execute a classifier that has been trained (i.e. pre-configured) to classify a window of data (for example, the data can be any sensor data or the corresponding features disclosed herein, for example, the harmonic proportions and/or phases for each beat) into a meal class or non-meal class by detecting certain properties of the data in that window. In the meal class, the training examples employed to configure the classifier are meals with the meal start time aligned to a pre-set time position in the window (for example, at about 20 minutes into a window, where the total window is about 140 minutes in length). Thus, as the window is repeatedly classified by processing circuitry 56 at incremental time steps (e.g. about every 1 minute), the window that is classified as the meal class and has the strongest response for the meal class represents the best alignment with the start of a meal. In other words, processing circuitry 56 classifies a rolling window of sensor data until it classifies one or more instances of the windows as corresponding to the meal class; the window that maximizes the response of the meal-class classifier represents the best alignment with the start of a meal. Processing circuitry 56 is therefore configured to select a meal start time based on the best matching window (e.g. 20 minutes into the best matching window). In one embodiment, the classifier can be a Feed-Forward Neural Network (FFNN; which could be a Convolutional Neural Network, further described below).

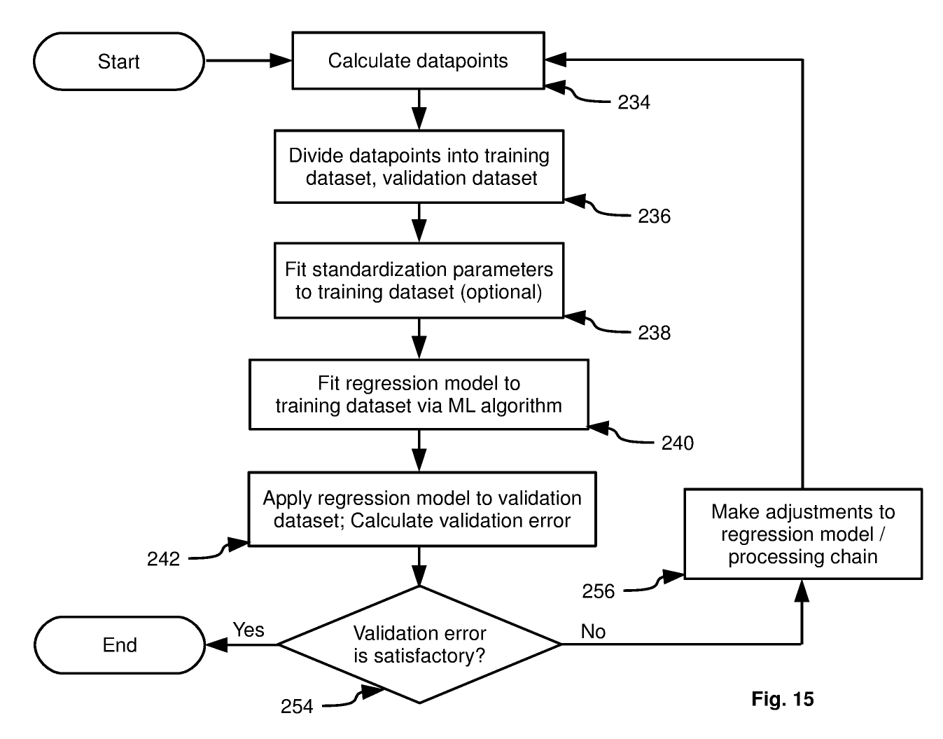

Furthermore, the FFNN could share the first one or more layers in common with the regression model 206 (for example, with the same alignment in time to the start of the meal) in order to improve statistical power (which results in better predictive performance for a limited size dataset) and/or reduce computation requirements. The classifier (or any classification model mentioned herein for predicting of nutrition-related metrics) can be trained by the same training process described below (with reference to FIG. 15), with the appropriate modifications (e.g. classifier model instead of regression model 206, dataset containing outputs (also known in the art as “targets” or “labels”) for a meal class and a non-meal class, etc.)

In an embodiment, the Recognition using Regions technique (GU, C et al., 2009) can be applied by processing circuitry 56 to detect the occurrence of meals and the meal start times (with the 2-dimensional inputs simplified to be 1-dimensional). As a further example, the Regions with CNN (R-CNN) technique (GIRSHICK, R et al., 2014) can be applied by processing circuitry 56 to detect the occurrence of meals and the meal start times (with the 2-dimensional inputs simplified to be 1-dimensional).

In the embodiments where a sliding window approach is used (such as the techniques disclosed above) the results of calculations that are repeated between overlapping window evaluations can be reused by processing circuitry 56 in order to reduce computational requirements and/or improve response time. Exemplary techniques are provided in (IANDOLA, F et al., 2014), which can be adapted to the present specification by simplifying the inputs to be 1-dimensional.

In certain embodiments, the detection of meals and calculation of meal start times and can be treated as a regression problem, where the input is a window of data (e.g. the last about 4 hours of data, for example the data being the harmonic proportions and/or phases of beats in the pulse profile signal described above) and one or more outputs representing the meal start time(s) (for example, implemented by output neurons with linear activation function in a FFNN), and optionally, one or more outputs representing the corresponding confidence score(s) (with range [0,1]) for each estimate of meal start time (for example, implemented by output neurons with sigmoid (e.g. logistical function) activation function in a FFNN). For example, the DeepMultiBox approach (ERHAN, D et al., 2014) can be used, with the inputs simplified to be 1-dimensional, and with the box simplified to be the time coordinate of a meal start (or the time coordinates of an interval containing the ingestion and/or digestion of a meal).

In an embodiment, one or more of the techniques disclosed above can be combined for detection of meal occurrence and calculation of meal start time. For example, an initial meal detection step using simple thresholding on sensor features (e.g. on the HRV values, as mentioned above) can be followed, in the event that a meal is likely to be present in a given window, by a more computationally costly meal start calculation based on a classifier or regression model, in order to reduce computation on average.

In an embodiment, if a meal start time is determined to be too close to another (previous, following, or either) meal start time (for example, the difference in meal start times is less than a threshold e.g. about 1.5 hours), the calculated nutrition-related metrics can be rejected or flagged by processing circuitry 56 as unreliable e.g. on the user interface 58. In certain other embodiments, a separate regression model 206 can be applied for the case of closely spaced meals, for example meals within an about 2 hour period (e.g. corresponding to “grazing” and/or multi-course meals) can be assessed as a single meal by regression model 206 for the prediction of corresponding nutrition-related metrics for the single “meal”.

Regression Model

In certain embodiments, with reference to FIG. 9, the processing circuitry 56 is configured to implement a regression model 206, which accepts the previously calculated features (from block 204) for a given meal (or a given window of time) and outputs a prediction of the desired nutrition-related metric(s), for example the total caloric intake for the meal (or given window of time). The regression model 206 is a program executed by processing circuitry 56 that is generated by application of a machine learning algorithm according to a training process as described below (with reference to FIG. 15). The machine algorithm can be any of a number of supervised learning algorithms, for example: Linear Regression (DRAPER, N R et al., 1998) (RIFKIN, R M et al., 2007), Random Forest Regression (BREIMAN, L, 2001), Feed-Forward Neural Networks (FFNN) (HAYKIN, S, 1998) (e.g. with a linear activation function on the output unit(s)), Gaussian Process Regression (RASMUSSEN, C E et al., 2006) or, in the presently preferred embodiment, Support Vector Regression (SVR) (CHANG, C C et al., 2011). The machine learning algorithm(s) (and/or any part of the training process, described below) can be executed “on-device” e.g. by processing circuitry 56, or “off-device” e.g. by processing circuitry 56′ (processing circuitry 56′ is further described below, with reference to FIG. 15). Depending on the particular implementation of regression model 206, the features may need to be standardized by processing circuitry 56, for example by applying Z-score scaling so that the data points for a given feature have zero mean and/or unit standard deviation. In addition, the output of the regression model 206 may require standardization, for example by applying Z-score scaling to target measurements during training, and reversing the scaling during predictions. In this embodiment, the Z-score parameters are determined during the training process and pre-configured in processing circuitry 56, as described below (with reference to FIG. 15).

(Note: In the embodiments of the present specification where a classifier model is used, the classifier equivalents of the regression models of the previous paragraph can be used, for example: logistic regression (DRAPER, N R et al., 1998), FFNN classification (HAYKIN, S, 1998) (e.g. with a logistic activation function on the output neurons), or Support Vector Classification (SVC) (CHANG, C C et al., 2011), etc.)

In one embodiment, regression model 206 can be a complex regression model (for example, complex linear regression (HUBER, W A, 2013)) applied to the complex Fourier coefficients (e.g. the time-pooled complex harmonic proportions, where the time-pooling windows can be defined the same as before, and the complex harmonic proportions are calculated by dividing the complex harmonic coefficient by a0 (or total power, etc.) for a given beat) e.g. in order to more effectively utilize the harmonic amplitude and phase information, improving prediction accuracy for a given training dataset size. In this embodiment, the output values are the real-valued nutrition-related metric(s). For example, the regression model of Equation 4 can be used:

y=β₀+xβ+ε, (4)

Where: y is the (complex-valued) output variable; x is the (complex-valued) vector of input one or more input variables, β₀ is the (complex-valued) y-intercept (e.g. determined by the training process), β is the (complex-valued) vector of one or more slopes (e.g. determined by the training process), and E is the error in the model. For example, y=a+ib, where a is the real component of the output variable, b is the imaginary component of the output variable, and i=\sqrt{−1}. For example, for predicting a real-valued nutrition-related metric (such as caloric intake): during training, a can be set to the nutrition-related metric and b set to 0; during predictions, the predicted a can be used as the nutrition-related metric (and the predicted b can be ignored). In other related embodiments, other methods of complex regression can be used, such as complex support-vector-machine regression e.g. (BOUBOULIS, P et al., 2013) or complex neural networks e.g. (ZIMMERMANN, H G, et al., 2011).

In an embodiment, regression model 206 can be implemented by processing circuitry 56 as two or more regression models, each trained and then combined while calculating predictions e.g. by the use of averaging (known as “model averaging”). In this embodiment, the two or more regression models do not necessarily need to be trained by an identical training algorithm, or by using an identical training dataset.

In certain embodiments, regularization such as L1 and/or L2 regularization can be applied during training of the regression model(s) in order to reduce overfitting (and thus improve prediction performance). In the case where one or more FFNNs are used, dropout regularization (HINTON, G E, SRIVASTAVA, N et al., 2012) can be applied (including in the fully-connected layers of a Convolutional Neural Network, described later) in order to reduce overfitting (and thus improve prediction performance). Furthermore, Rectified Linear Units (ReLU) can be used as the activation functions in any FFNN of the present specification. Finally, a sparsity penalty can be applied (for example, on the activities of the neurons) in order to reduce overfitting/improve prediction performance in a FFNN.

Training Process

FIG. 15 is a flowchart which illustrates an exemplary training process for determining the regression model 206 from a dataset, for example involving selection of regression model class/machine learning algorithm, fitting of regression model parameters, hyperparameters, choice of preprocessing/features, and any other configuration of the processing chain. (As mentioned earlier, for the purposes of this specification, a “dataset” is a collection of datapoints, and each “datapoint” is a (features measurement, nutrition-related parameter measurement) pair.)

First, the datapoints are calculated (block 234). The collection of data from a given user (e.g. start of meal time (or start of pre-set window), corresponding nutrition-related metric measurements, and/or corresponding sensor data) required to calculate the datapoints can be performed by the user (or in general, an operator) by use of portable device 50 and/or an external device such as a mobile phone and/or website; exemplary means for data collection are further described below under “Calibration”. Note: the calculation of datapoints (block 234) and/or any other part of the training process can occur “off-device” (e.g. on processing circuitry 56′ further described below). Next, the datapoints are divided into a training dataset and a validation dataset (block 236). Next, if required for the particular regression model 206 being trained, standardization is applied to the all datapoints, where the standardization parameters are fit to the training dataset (block 238). Next, the regression model 206 is fit to the training dataset (block 240). Next, the regression model 206 is applied to the validation dataset (specifically, to the features measurements of the validation dataset), producing nutrition-related metric predictions given the features measurements; the validation error is calculated as error between nutrition-related metric predictions and the nutrition-related metric measurements of the validation dataset (block 242). For example, the Mean Absolute Error (MAE) can be used (or the Mean Squared Error, etc.). (Alternatively, any desired objective function(s) can be calculated in this step to be optimized during the training process.) If the resulting validation error is considered satisfactory (block 254), the training process is completed, and the regression model parameters are loaded onto processing circuitry 56. Otherwise, adjustments can be made to the processing chain (e.g. adjusting hyperparameters for regression model 206, etc.) (block 256), and the training process repeated, e.g. restarting from block 234. (In order to speed up the calculations, one can reuse results/skip calculations that have not changed from the previous iteration.)

For example, in one embodiment, relative error regression can be applied during the training process, where the objective function to be minimized is the relative error, for example Least-Squares Relative Error (LSRE) regression (SHAMMAS, N, 2013). This embodiment has the potential advantage of resulting in a model with less prediction error for smaller meals, where a given absolute magnitude of error may be more noticeable than for a larger meal.

In certain embodiments, a robust regression model can be used for regression model 206, where the robust regression model is less sensitive to outlier datapoints. In particular, a robust regression machine learning algorithm can be applied during training (for example, the Least Trimmed Squares Robust (High Breakdown) Regression, e.g. implemented as the “ltsReg” method from the “robustbase” package in the R statistical language).

There are a number of variations on the exemplary training process which can be used. For example, cross-validation can be used in order to more effectively use a limited size dataset for calculating the validation error. For example, Leave-One-Out Cross-Validation (LOO-CV) can be used.

Additionally, Bayesian optimization can be applied to tune the settings for the processing chain (including hyperparameters of regression model 206) in order to minimize the validation error (SNOEK, J et al., 2012b). This approach can reduce the need for hand-tuning when there are many hyperparameters to tune and/or the practitioner does not have extensive experience in tuning a given regression model class. An exemplary implementation of Bayesian optimization applied to hyperparameter tuning is the Spearmint software package (SNOEK, J, 2014), and another example is the hyperopt software package (BERGSTRA, J et al., 2013).

Finally, a test dataset could be selected (not shown in FIG. 15) from the datapoints calculated at block 234, apart from the training dataset and validation dataset, to be used to calculate the “test error” after completing the process of FIG. 15, in order to get an unbiased (or minimally biased) estimate of the true prediction error for the given dataset.

Calibration

While the training process (e.g. FIG. 15) enables the determination of a working configuration of the processing chain, further strategies can be employed to enable the processing chain to work more effectively for a given user (that is, to enable processing circuitry 56 to generate more accurate outputs for a given user by executing the processing chain), in particular a new user (e.g. one who has recently purchased the portable monitoring device 50 as a product) for whom limited data is available. All other factors being equal, it is preferable that user intervention (e.g. for collecting of data) is kept to a minimum in order to reduce inconvenience for the user. In the cases where labelled data (e.g. with the time of meal start and measurement of nutrition-related metrics of the meal, or in the case of predicting a nutrition-related metric for a window of time, with measurement of nutrition-related metrics for the window) is collected from the user, the portable monitoring device 50 can receive (at processing circuitry 56), through user interface 58, input data from the user defining meal (or window of time) information (for example, time of meal start and the nutrition-related metrics for the meal; similarly for a window of time). In certain embodiments, where the automatic detection of meal start (or window start) does not need user-specific training or configuration, the time of meal start (or time of window start) can be provided by the automatic detection of meal start (or window start) instead of being provided by the user. Furthermore, an application on an external device (e.g. an application on a mobile phone or application on a website) can be used to collect the meal information (or window of time information) from the user. Existing applications can be used, for example the “MyFitnessPal” mobile application (for iOS platform from Apple Inc., Android platform from Google Inc, and web browser) (MyFitnessPal, 2014). As an alternative or complement to the user choosing a meal and specifying the nutrition-related parameters, the portable monitoring device 50 (or related application on an external device) can dictate to the user (e.g. via user interface 58) specific calibration meals stored in a memory of device 50, for which the nutrition-related metrics are known (e.g. pre-configured in memory at device 50 or the external device), and optionally, the user can choose or specify a specific serving size of said calibration meal.

In the preferred embodiment, about 3 or more weeks of labelled meal datapoints are collected from a new user. The regression model 206 is then fit to this dataset by applying the machine learning training algorithm. In a variant of this embodiment, unsupervised (or semi-supervised) pre-training (further described below) can be applied to more effectively use the labelled data (e.g. reduce overfitting/reduce prediction error), and/or enable the use of further collected unlabelled data (i.e. where only the features measurements are available). In this variant, the regression model can be repeatedly fit to additional data (e.g. labelled and/or unlabelled data) as it arrives (e.g. as it is collected by device 50 during use by the user), or on a pre-set schedule e.g. about every week.

In another embodiment, regression model 206 is a multiple-user model, trained using a dataset containing data from one or more users (for example, about 30 users). If user-wise cross-validation (i.e. where data from any given user is present in either the training subset or the validation subset, but not both) gives a satisfactory validation error, this approach can be applied to a new user without any further calibration. Otherwise, the multiple-user model can be adapted to a new user by any of a number of methods. For example, labelled and/or unlabelled data from the new user can be incorporated into the multiple-user model by retraining a new regression model with the multiple-user dataset augmented with data from the new user. In another embodiment, given a dataset of labelled meals from the new user (e.g. for about 1 week), a single variable linear regression can be applied on top of the multi-user regression in order to correct predictions made by the multiple-user model, where the input variable is the prediction made by the multiple-user model, and the output variable is the corresponding label for the desired nutrition-related metric. In another embodiment, the multiple-user model can be adapted to the user (e.g. a new user) by adding an offset to the predictions made by the multiple-user model which is the difference between the mean value of the nutrition-related metric measurements (e.g. mean kcal of meals) for the user and the mean value of the nutrition-related metric measurements for the training dataset used to train the multiple-user model. As a further example, the mean value of the nutrition-related metric measurements for the user can be estimated from a food journal (e.g. over about 2 days or about 7 meals) and/or estimated from demographic information (e.g. age, gender, height, body fat percentage, diabetes status, cardiovascular disease status, and/or weight).

In certain embodiments, demographic information from the user (e.g. age, gender, height, body fat percentage, diabetes status, cardiovascular disease status, and/or weight) (e.g. obtained via user interface 58 and/or an application on an external device such as a mobile phone and/or website) can be used to improve predictions by the regression model 206. For example, a particular regression model can be selected based on certain demographic information (for example, one regression model may be trained on males, and then used for predictions on males, and similarly for females). As another example, demographic information can be used as feature(s) in the regression model 206 during training and predictions, in order to improve prediction performance.

In an embodiment, the caloric intake calculations can be automatically calibrated by employing caloric expenditure data and making the assumption that (over a window of time, e.g. for a given day or in aggregate for a number of days):

Cᵢₙₜₐₖₑ=C_{expenditure}, (5)

Where: Cᵢₙₜₐₖₑ is the caloric intake, and C_{expenditure} is the caloric expenditure, e.g. due to metabolic activity such as exercise and the basal metabolism. In this embodiment, for example, a single-variable linear regression can be performed where the total predicted caloric intake for two or more windows of time are fitted to the corresponding C_{expenditure} values as expressed in Equation 5. The output of this calibration can be scaling and/or offset factor(s) applied to the caloric intake predictions for each given meal (or window of time, where nutrition-related metrics are being predicted for said window of time). C_{expenditure} can be determined by processing circuitry 56 based on demographic information and/or input data received from one or more sensor(s) 54, for example a heart rate sensor (for example, based on a pulse profile sensor 52 such as PPG or ECG) or a motion sensor (for example, an accelerometer), or any metabolic rate sensor.

As an example, the technique of Equation 4 can be augmented by employing data representative of the user's weight over a window of time (for example, a number of days), for example according to the relationship expressed as:

Cᵢₙₜₐₖₑ−C_{expenditure}=(M_{end}−Mₛₜₐᵣₜ)*K, (6)

Where: Cᵢₙₜₐₖₑ is the total caloric intake [kcal], C_{expenditure} is the total caloric expenditure [kcal], Mₛₜₐᵣₜ is the user's body mass at the start of the given window [lbs], M_{end} is the user's body mass at the end of the given window, and K is a constant (for example about 3555 [kcal/lb]).

Means for reducing the computation during training can be implemented, which can be especially valuable when retraining the regression model 206 frequently and/or when training the regression model 206 in a system serving many portable monitoring devices 50 (e.g. 100's or more), or on a system in a resource-constrained environment (e.g. “embedded” environment, such as a battery powered monitoring device 50 or a mobile phone). As an example, instead of a FFNN trained by back-propagation gradient descent, an Extreme Learning Machine (ELM) (HUANG, G B et al., 2006) can be used. As an example, in the case of unsupervised learning (e.g. for unsupervised feature learning or unsupervised pre-training), ELM trained as a Stacked Auto-encoder (CAMBRIA, E et al., 2013) can be used, and/or a Marginalized Corrupted Features (MCF) model can be applied (e.g. marginalized Stacked De-nosing Auto-encoder (mSDA)) (MAATEN, L et al., 2013).

Predicting Other Nutrition-Related Metrics

As described above, in certain embodiments, other nutrition-related metrics besides caloric intake are predicted by processing circuitry 56 (either in combination with or in lieu of caloric intake). For example, in one embodiment, in addition to caloric intake per meal (or, alternatively, per pre-set window of time, e.g. the last about 1 hour), the macronutrient intake (e.g. mass, volume, or caloric intake, (and/or mass proportion, volume proportion, caloric proportion) of: carbohydrates, proteins, and/or fats) per meal (or per pre-set window of time) are predicted by processing circuitry 56. For a given nutrition-related metric, the disclosed processing chain and training process (for example, with reference to FIG. 15) can be used, but with the output variable(s) set to be the desired nutrition-related metric(s), and accordingly obtaining the appropriate dataset (with labels for the desired nutrition-related metric(s)) for one or more user(s). Thus, a new processing chain is trained for predicting each nutrition-related metric.

In certain embodiments where multiple nutrition-related metrics are predicted, some or all of the sensors and/or features (e.g. the time-pooled harmonic coefficients) and/or any part(s) of the processing chain can be shared between processing chains to reduce computational requirements. In certain embodiments, part of the regression model 206 can be shared in common between the different nutrition-related metrics (output variables) to be predicted. For example, in a feed-forward neural network (FFNN), one or more hidden layers (that is, collections of intermediate features computed by processing circuitry 56) can be in common between output variables, with a separate output neuron (with linear activation function in the case of real-valued variables such as caloric intake or grams/calories of macronutrient intake) for each output variable. Optionally, additional separate hidden layer(s) for each output variable can be used after the common hidden layer(s). The advantage of this approach (known in the field as “multi-task learning”) is two-fold: it reduces computation costs during both the training process and when calculating predictions, and in some cases (as can be determined by the validation error) reduces overfitting/improves prediction accuracy (countering the effect of a smaller dataset) by sharing statistical power between the multiple regression models. In another embodiment, a common regression model 206 can be trained to predict other metric(s) simultaneously with the desired nutrition-related metrics, in order to gain the benefit of shared statistical power; these one or more other metric(s) are not necessarily nutrition-related metrics, and they are not necessarily used for further processing or for determining outputs from device 50. For example, the common regression model can be trained to predict one or more HRV parameters and/or the amount of physical activity for a given meal or a given window of time.

In one embodiment, a classification model can be used to predict a class (e.g. high-caloric meals, low-caloric meals, fat-dominant meals, etc.) used to select one of several regression models 206 for predicting the desired nutrition-related metrics; said regression models 206 are optimized for the corresponding class (e.g. each trained with a dataset representative of the corresponding class).

In one embodiment, three regression models (or a single regression model with three outputs, as described above) are trained to predict for a meal (or for a pre-set window of time) mass of carbohydrates intake, mass of proteins intake, and mass of fats intake, from which the total caloric intake can be calculated according to the relation expressed as:

Cᵢₙₜₐₖₑ=4*m_{carbohydrates}+4*mₚᵣₒₜₑᵢₙₛ+9*m_{fats}, (7)

Where: Cᵢₙₜₐₖₑ is the caloric intake [kcal], M_{carbohydrates} is the mass of carbohydrates intake [grams], mₚᵣₒₜₑᵢₙₛ is the mass of proteins intake [grams], and m_{fats} is the mass of fats intake [grams]. The constants represent the Atwater general factors for Metabolisable Energy (ME); other variants can be used such as where 4*M_{carbohydrates} is replaced with a factor of 3.75 [kcal/g] for mass of available carbohydrates (e.g. monosaccharide) and a factor of 2 [kcal/g] for mass of unavailable carbohydrates; optionally a factor of 7 [kcal/g] (28.8 [kJ/g]) for mass of alcohol can be added; as a further example, the Net Metabolisable Energy (NME) factors can be used (for example, see the caption of FIG. 2 as well as Table 1 of (LIVESEY, G, 2001)).

Furthermore, alternative instances of deriving nutrition-related metrics from other nutrition-related metrics can be used by processing circuitry 56. For example, a nutrition quality metric can be calculated from other nutrition-related metrics, for example according to:

Q=f(GI,GL,PI,C_{sugar}/C), (8)

Where: Q is a quality metric, f(•) is a suitable function, GI is the glycemic index, GL is the glycemic load, PI is the phytonutrient index (MCCARTY, M F, 2004), C_{sugar} is the caloric intake due to sugar, C is the total caloric intake (each of which can be predicted by processing circuitry 56 via the execution of one or more regression models 206); not all input variables need be used by f(•) in this example. For example, f(•) can be chosen so that Q=100−GI. As another example, f(•) can be chosen so that Q=(GI+PI+100*(1−C_{sugar}/C))/3.

In an embodiment where hydration is calculated by processing circuitry 56 via the execution of regression model 206 (e.g. by use of datapoints with hydration labels), the relative over-hydration (“rel. OH”) can be determined by processing circuitry 56, as defined in the following equation:

rel. OH=OH/ECW, (9)

Where: OH is the absolute over-hydration (the difference between the user's actual ECW and the expected ECW, in units of volume, e.g. L), and ECW is the extracellular water, in units of volume, e.g. L. For example, in the presently preferred embodiment measurements of rel. OH and the corresponding features measurements can provided to train regression model 206 in order to predict rel. OH. In another embodiment, separate regression models 206 can be trained to predict OH and ECW, from which rel. OH can be calculated according to Equation 9.

In an embodiment, the hydration range category (or “class”) can be predicted (either by using a classifier for regression model 206 or by binning the output of regression model 206). For example, normohydration can defined as when rel. OH is between the 10th and the 90th percentile for healthy, age- and gender-matched individuals from the reference population, e.g., between 10th percentile (−7%) to 90th percentile (+7%), while volumes below and above this range can be defined as underhydration and overhydration, respectively (ZALOSZYC, A et al., 2013).

In an embodiment, the potassium/sodium (“K/Na”) ratio can be predicted by processing circuitry 56 in terms of a class, similar to the method described above for predicting a class of hydration. For example, the classes can include: K/Na<1/2 corresponding to “average”, 1/2<K/Na<5/1 corresponding to “better than average”, K/Na>5/1 corresponding to “healthy”. Similarly, classes can be predicted for ranges of other nutrition-related metrics such as sugar content (e.g. in terms of caloric proportion), glycemic index, phytonutrient index, antioxidant content, etc.

In obtaining datapoints for a given nutrition-related metric (e.g. for training of a regression model 206 used by processing circuitry 56 to predict that nutrition-related metric), nutrition-related metrics can be measured directly, and/or known values can be used (from an existing database) for food items consumed in the collection of data. For example in the case of glycemic index and glycemic load, an exemplary database is available at (NEUHOUSER, M L et al., 2006).

Features and Sensors Variations

As described above, the harmonic proportions/phases (e.g. with time pooling) can be used as features informative of nutrition-related metrics (e.g. caloric intake, macronutrient content, etc. for a given meal or window of time). In this paragraph, non-exhaustive supporting background from the nutrition science and physiology literature is provided to illuminate some of the mechanisms behind the use of these features, and thus provide context for the use of other features in the present specification. The provided supporting background and mechanisms behind the use of these features are exemplary and are not intended to limit the application of a feature (or set of features) to predicting only certain nutrition-related metrics (or health-related, etc.) metrics. Intake of a meal is known to have acute effects on physiology. For example, during anticipation and/or ingestion of food, there may be increases in heart, rate, cardiac output, and cardiac output, with typically minor changes in gastrointestinal vascular resistance. For example, the tasting and chewing of food is able to raise blood flow in the celiac artery, but typically not the superior mesenteric artery. For example, chewing is typically associated with as increases in cerebral blood flow and blood flow to muscles involved in chewing) as well as changes in autonomic state (e.g. as can be measured by HRV); similar effects may occur for swallowing. The ensuing ingestion of food causes a further rapid increase in blood flow through the celiac artery followed by a more gradual increase in blood flow through the superior mesenteric artery. For example, within minutes of ingestion of a meal (“postprandial state”), overall blood flow (e.g. cardiac output) is increased relative to the fasting (“preprandial”) state, with blood flow increases (to the splanchnic regions, e.g. the stomach, and later to the intestines and other visceral organs) which are dependent (e.g. positively correlated in magnitude and time duration) on caloric intake with an additional dependence (e.g. in magnitude, distribution by body location, and time course) on the macronutrient content (KVIETYS, PR, 2010) (HOLZER, P, 2012) (VAN BAAK, M A, 2008) (KEARNEY, M T et al., 1995) (WAALER, B A et al., 1991) (BRUNDIN, T et al., 1994) (HAWLEY, S K et al., 1992) (WAALER, B A, et al., 1992). For example, any sensor(s) that measure these and/or other physiological effects (or one or more variables correlated to these effects) can be used as physiological sensor(s) 54, as will be further described below. For example, these and/or other physiological effects can be quantified by parameters calculated from the pulse profile, for example including the Augmentation Index (AIx) (PHILLIPS, L K et al., 2010) (LITHANDER, F E et al., 2013) and the harmonic proportions (WANG, W K et al., 1996) (YIM, Y K et al., 2011). (These and/or other physiological effects can also be quantified by sensors besides the pulse profile, as described further below.) The pulse profile is known to be dependent on additional nutrition-related metrics, including: blood glucose concentration (HOFFMAN, R P et al., 1999); glycemic index and/or glycemic load (and thus carbohydrate “quality”, e.g. sugar-like or starch-like) (REGIO-RODRIGUEZ, J I et al., 2014) (JENKINS, A et al., 2010); blood lipids concentration (e.g. blood triglycerides (NESTEL, P J et al., 2001), for example where the pulse profile is known to be affected due to inflammation (BLANN, A D et al., 2013) (VLACHOPOULOS, C et al., 2005)); fat quality (ESSER, D et al., 2013); protein quality (HOLMER-JENSEN, J et al., 2003); state of hydration (KWAN, B C et al., 2014) (HOGAS, S et al., 2012); sodium intake (DICKINSON, K M et al., 2014) (LIU, Y P et al., 2013); potassium intake (BLANCH, N et al., 2014) (LENNON-EDWARDS, S et al., 2014); potassium/sodium ratio (LENNON-EDWARDS, S et al., 2014); caffeine intake (HSU, T L et al., 2008); alcohol intake (MAHMUD, A et al., 2002) (SHIMIZU, Y et al., 2010); and phytonutrient intake or absorption (KHOR, A et al., 2014) (e.g. as measured by a phytonutrient index, or by change in total plasma antioxidant capacity, as described above), for example where the pulse-profile is known to be affected by post-meal changes in oxidative stress and/or inflammation (KALS, J et al., 2008).

In certain embodiments, with reference to block 212 of FIG. 10, other features besides harmonic coefficients can be generated by processing circuitry 56 to summarize the pulse profile, either in combination with or in lieu of the harmonic coefficients. For example, these features can summarize information in beat timing (such as HR and/or HRV features), beat amplitude, pulse profile baseline, beat “shape” or morphology (such as time-based, frequency based (e.g. non-parametric (e.g. fast Fourier, discrete cosine, or wavelet transforms), parametric (e.g. autoregressive model)), time-frequency based (short-time Fourier transform), etc. Furthermore, these features can also be time-pooled (for example, into one or more averaged windows timed with reference to the meal start time) in a manner similar to that described above with the harmonic coefficients (e.g. according to Equation 1 and Equation 2).

For example, one or more time-domain features such as the features described in (ELGENDI, M., 2012) can be used (that is, generated by processing circuitry 56 based from the pulse profile data). For example, these features include: Systolic Amplitude, Pulse Width (the pulse width at the half height of the systolic peak), Pulse Area (the total area under the foot-amplitude-subtracted pulse profile curve for a given pulse), Inflection Point Area ratio (IPA=diastolic area divided by systolic area), Augmentation Index (AIx), ΔT (time between the systolic peak and the diastolic peak), Stiffness Index (body height divided by ΔT) (and/or the related Pulse Wave Velocity (PWV) e.g. measured by one or more pulse profile sensors e.g. using an ECG sensor and a PPG sensor), Crest Time (time from the foot of the pulse profile waveform to its peak). Additionally, features derived from the second derivative pulse profile (e.g. PPG) include: ratio b/a, ratio c/a, ratio d/a, ratio e/a, ratio (b−c−d−e)/a, ratio (b−e)/a, ratio (b−c−d)/a, and ratio (c+d−b)/a; where: a, b, c, d, and e are the absolute amplitudes at the 1^{st}, 2^{nd}, 3^{rd}, 4^{th}, and 5^{th} local minima or maxima of the second derivative pulse profile (e.g. PPG), respectively (ELGENDI, M., 2012). (Note: in the case of digital signal processing, the second derivative can be substituted with the second finite difference.)

The heart-rate-adjusted Mx can be used (AtCor Medical Pty Ltd), for example, according to the relationship expressed as:

AIx@75=AIx+0.48*(HR−75), (10)

Where: AIx@75 is the Augmentation Index adjusted to a heart rate of 75 bpm [%], AIx is the original Augmentation Index[%], 0.48 [%/bpm] is an exemplary constant obtained from measurement, HR is the heart rate [bpm]; or alternatively, according to the relationship expressed as:

AIx@75=AIx*(HR/75), (11)

Where: AIx@75, AIx, and HR are as defined for Equation 10.

For example, one or more of the time-domain features described in (YIM, Y K et al., 2014) can be used, e.g. h1, h2, h3, h4, h5, T (pulse period), t1/T, t2/T, t3/T, t4/T, t5/T, W/T, Ap (pulse area), As/Ap, Ad/Ap, W area (Aw), and Aw/Ap.

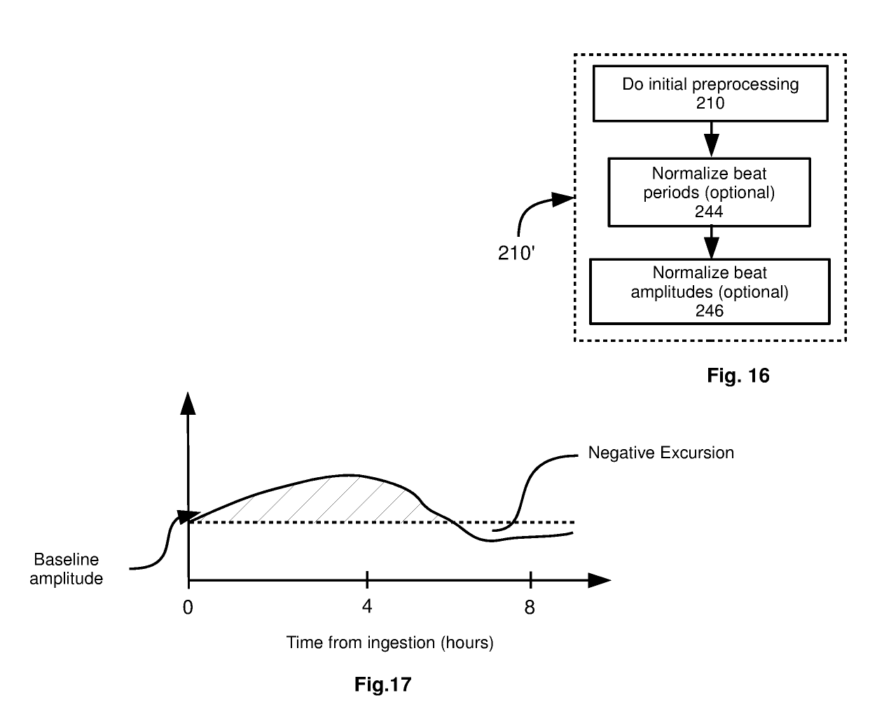

For example, time-frequency domain features can be used, such as a short-time Fourier transform (STFT) (including windowed variants) coefficients, or wavelet transform coefficients (including wavelet transforms that are an over-complete basis). Referring to FIG. 16, these time-frequency features can be calculated with additional preprocessing (following the preprocessing 210 as described in FIG. 12), for example involving an additional step to normalize the period for a given beat to a fixed number of samples (e.g. 100 samples) (block 244) (for example, by the use of Fourier transform resampling) and/or an additional step to normalize the amplitude (block 246), for example by dividing the signal by the mean value (i.e. a₀ as described earlier) of a given beat period or by the RMS amplitude (after subtracting the mean) of a given beat period, or RMS of the harmonic amplitudes for harmonics 1 and above), or peak-peak amplitude of a given beat period. These time-frequency domain features can be calculated by processing circuitry 56 and used as features for regression model 206, with the time-frequency transform applied within a single beat (for example, the input window for the time-frequency transform can be the period of a single beat, for example as indicated in FIG. 8); alternatively, the input window for the time-frequency transform can be a pre-set window of time that can include multiple beats (e.g. about 10 beats, or about 60 seconds).

For example, other features derived from a pulse profile signal can include the baseline (also known as the “DC component”), the beat amplitude (e.g. average amplitude or a₀ as described above, or the RMS amplitude after subtracting the mean, the RMS of the harmonic amplitudes for harmonics 1 and above, or the peak-peak amplitude), the beat period (or heart rate), or Heart Rate Variability (HRV) (further details and physiological background are given below). Additionally, parameters related to cardiac output and/or stroke volume can be calculated from the pulse profile by processing circuitry 56 (WANG, L et al., 2009) and used as features for the regression model 206 (with some physiological background given above), for example according to relationship expressed as:

$$CO \text{∼} IHAR = \frac{1 - {\sum }_{n = 2}^{N} {a}_{n}^{2} / {\sum }_{n = 1}^{N} {a}_{n}^{2}}{IPA} \qquad ( 12 )$$

Where: CO is the cardiac output [volume/time e.g. L/min], IHAR is the Inflection and Harmonic Area Ratio which is approximately linearly proportional to CO, aₙ is the n^{th} harmonic amplitude (equivalently, hpₙ can be used), and IPA is the Inflection Point Area ratio (=diastolic area of a given beat/systolic area of a given beat, where diastolic and systolic regions are separated by the inflection point) e.g. as described above herein. As another example, either the numerator of Equation 12 (which is strongly correlated to the systolic and diastolic blood pressure) and/or IPA (which is a good indicator of TPR) can be used as features.

As another example, features related to gastric motility can be calculated by processing circuitry 56 from the pulse profile (YACIN, S M et al., 2010) and used as features for the regression model 206 (further physiological background is given below).

In certain embodiments, an Electrocardiogram (ECG) sensor can be used as a “pulse profile” sensor, with all descriptions herein applying to a pulse profile sensor 52 (e.g. to a PPG sensor) also applying to the ECG sensor, unless otherwise provided. In this embodiment, features such as those specified in (NATARAJAN, A et al., 2013) can be calculated by processing circuitry 56 from the ECG data and used as features in regression model 206. In another embodiment, a phonocardiogram (PCG) sensor and/or stethoscope or stethophone can be used as a “pulse profile” sensor 52.

In certain embodiments, the physiological sensor(s) 54 can measure data representing aspects of physiology besides the pulse profile. In this case, the output of a given sensor 54 can be used as feature(s) (or as data for calculating feature(s)) for the regression model 206, either in combination with or in lieu of features calculated from the pulse profile. Additionally, the sensors 54 are preferably non-invasive (not requiring penetration of the user's skin), but invasive sensors can be used. Additionally, data from sensors on an external device (for example, a mobile phone) can be used for the techniques in the present specification. For example, any sensors 54 measuring the blood flow (e.g. its magnitude, spatial distribution in the body, and/or time evolution, or correlates thereof) can be used; for example see Chapter 1 of (PHUA, C T, 2012). For example, one or more temperature sensor(s) can measure the effects (such as skin temperature) and/or distribution of blood flow throughout the body. For example, a thermal camera can measure the effects (such as skin temperature) and/or distribution of blood flow on the body. For example, tissue metabolite concentration sensors can be used; for example a blood glucose concentration sensor (and/or interstitial glucose concentration sensor (KULCU, E et al., 2003)) can be used as a physiological sensor 54. Example implementations of a blood glucose concentration sensor are contained in the paper (MONTE-MORENO, E, 2011), in U.S. patent application Ser. No. 13/128,205, and in U.S. patent application Ser. No. 13/991,034. Additionally, a blood triglycerides concentration sensor (and/or interstitial triglycerides concentration sensor (PARINI, P et al., 2006)) can be used; for example, the techniques referenced in the previous sentence can be used, where blood glucose concentrations are replaced with blood triglyceride concentrations when training and testing the stochastic estimator.

Additionally, in one embodiment, one or more of the features described in the paper (MONTE-MORENO, E, 2011) (e.g. which can be calculated by processing circuitry 56 from data provided by pulse profile sensor(s) 52) can be used as features for regression model 206; the relevant sections for calculating the following features are incorporated herein by reference: KTE_{AR}, KTE^{μ}, KTE^{σ}, KTE^{iqr}, KTE^{skew}, HR^{μ}, HR^{σ}, HR^{iqr}, HR^{skew}, AR_{PPG}, OSR_{PPG}, HS^{μ}, HS^{σ}, HS^{iqr}, HS^{skew}, Log E^{AR}, Log E^{σ}, Log E^{irq}. For example, the instantaneous (i.e. per each beat) beat period (or heart rate) can be determined for a period of time (e.g. about 1 minute), from which the mean (HR^{μ}), standard deviation (HR^{σ}), interquartile range (HR^{iqr}), and skewness (HR^{skew}) can be determined by processing circuitry 56 as features for regression model 206; additionally the first N coefficients of the autoregressive spectrum of the pulse profile over a given period (e.g. about 1 minute) (AR_{PPG}), where N is an integer (e.g. 5, or 12) can be used as features for regression model 206; additionally, the Teager-Kaiser energy operator can be applied to the pulse profile, from which the autoregressive spectrum coefficients (KTE_{AR}), and/or mean (KTE^{μ}), standard deviation (KTE^{σ}), interquartile range (KTE^{iqr}), or skewness (KTE^{skew}) are calculated as features for regression model 206. Additionally, one of more of the features described in U.S. patent application Ser. No. 13/991,034 (U.S. Publication No. 2013/0267796) (e.g. which can be calculated from data provided by pulse profile sensor(s) 52) can be used as features for regression model 206; the relevant sections for calculating the following features are incorporated herein by reference: CEPS_{signal}, CEPS_{HR}, CEPS_{Energy}, Log E^{v}, Log E^{a}, E^{skew}, CEPS_E^{u}, H^{s}, CEPS_H^{s}, HR^{u}, HR^{a}, HR^{skew}, CEPS_HR^{u}.

Other physiological sensor(s) 54 can be used to provide feature(s) (e.g. for the regression model 206). For example, sensors that measure cardiovascular parameters such as cardiac output, heart rate, stroke volume, mean arterial blood pressure, systolic blood pressure, diastolic blood pressure, arterial compliance, and/or peripheral resistance can be used (with some physiological background given above). As another example, sensors measuring the Thermic Effect of Food (and/or “Specific Dynamic Action” and/or “Diet-Induced Thermogenesis”) (VAN BAAK, M A, 2008) (WESTERTERP, K R et al., 2004) (MCCUE, M D, 2006) (KUO, C D et al., 1993) can be used, e.g. metabolic and/or respiratory sensors, e.g. metabolic rate, respiration frequency, respiration tidal volume, consumed volume of oxygen (VO₂), eliminated volume of carbon dioxide (VCO₂), respiratory quotient (VCO₂/VO₂), arterial blood O₂ saturation, venous blood O₂ saturation, blood gas tension (e.g. PₐO₂, PᵥO₂, PₐCO₂, PᵥCO₂), or HRV (MILLIS, R M et al., 2011). For example, respiration rate, respiration tidal volume and/or thoraco-abdominal separation can be measured via the pulse profile (MEREDITH, D J et al., 2012); arterial blood O₂ saturation can be measured by pulse oximetry, and venous blood O₂ saturation can be measured by spiroximetry (FRANCESCHINI, M A et al., 2002). For example, HRV parameters can be calculated from the pulse profile as mentioned above and described in further detail below. Any sensors measuring nervous system state (e.g. central, peripheral, autonomic, etc.) can be used; for example sensors measuring the state of the Autonomic Nervous System can be used, such as skin temperature, skin conductance, HRV (YACIN, S M et al., 2009) (LIPSITZ, L A et al., 1993) (including HRV parameters calculated from the pulse profile, as mentioned above), as well as sensors that measure the state of the Central Nervous System and/or brain state, such as Electroencephalography (EEG).

In further detail, there are a number of parameters for quantifying HRV given heart rate data or beat period data (whether the heart rate data was obtained from a pulse profile sensor 52 or some other physiological sensor(s) 54). For some examples, see (VON BORELL, E et al., 2007). For example: time-domain HRV parameters such as pNNx (percent of successive N-N differences greater than “x” ms, where x could be about 30 ms, about 50 ms or about 80 ms, for example), SDNN (Standard Deviation of N-N intervals) or SDNN after low-pass filtering (e.g. with an about 0.15 Hz cutoff), RMSSD (Root Mean Square of Successive Differences) can be used (or their robust equivalents: Inter-quartile range of N-N intervals (including after low-pass filtering with a moving average), Root Median Square of Successive Differences); frequency domain HRV parameters (based on calculating a power density spectrum of the heart beat periods, e.g. by parametric or non-parametric methods, and potentially with the use of a Lomb-periodogram method to handle the unevenly sampled time-series) such as: VLF (Very Low Frequency: less than about 0.04 Hz), LF (Low Frequency: about 0.04 Hz-about 0.15 Hz), HF (High Frequency: about 0.15 Hz-about 0.40 Hz), LF/HF, Total power can be used; nonlinear HRV parameters (PERKIÖMÄKI, J S et al., 2005) such as the exponents of fractal scaling (e.g. short-term fractal scaling exponent α1, long-term fractal scaling exponent α2, long-term scaling slope β), ApEn (Approximate Entropy), SampEn (Sample Entropy) can be used.

As another example, sensors 54 can include sensors measuring gastric parameters (e.g. parameters relating to gastric motility, gastric distension, gastric emptying, gastric muscle tone, etc.) to be used by processing circuitry 56 to provide features, such as an Electrogastrogram (EGG) (YACIN, S M et al., 2010). Gastric parameters can also be derived by processing circuitry 56 from the pulse profile, as mentioned above. For example, the power ratio (e.g. postprandial power/preprandial power for the dominant frequency of EGG) and gastric emptying time are known to be positively correlated with meal size; these and other gastric parameters (such as fundamental frequency of gastric contractions) have a further dependence on meal composition (e.g. macronutrient content, sodium content, etc.) (RIEZZO, G et al., 2013) (GONLACHANVIT, S et al., 2001) (AVIV, R et al., 2008). Sensor(s) 54 measuring bioimpedance (or in general, body composition, e.g. local to digestion, as in directly measuring the composition changes about the gastrointestinal tract, and/or non-local to digestion, e.g. measuring body composition changes in the proximity of portable device 50 not necessarily reflecting the gastrointestinal tract, e.g. body composition changes of wrist tissue) can be used to provide input to processing circuitry 56 for generating features (e.g. resistance for given spectral bands of frequency) that can be predictive of (among others) hydration (e.g. rel. OH as described above) and/or caloric intake (DEHGHAN, M et al., 2008). Exemplary bioimpedance parameters which can be used as features in regression model 206 are provided in U.S. Pat. No. 8,374,688, entitled “System and methods for wireless body fluid monitoring”, Libbus, I and Bly, M, in particular the features involved in the means for measuring R(ICW) and/or R(ECW): the text from column 10 lines 5 to 60 (and any referenced figures) and from column 12 lines 31 to 59 (and any referenced figures), which are incorporated herein by reference. For example, R(ECW) (resistance due to extracellular fluid) can be effectively measured with frequencies in a range from about 0.5 kHz to about 20 kHz, for example from about 1 kHz to about 10 kHz. For example, features based on bioimpedance (such as, but not limited to those disclosed herein) are known in the art to be correlated to hydration (e.g. rel. OH as described above) as well as caloric and/or macronutrient intake.

In certain embodiments, one or more sensor(s) 54 measuring hormone concentrations and/or their effects can be used as features for regression model 206 for the prediction by processing circuitry 56 of nutrition-related metrics. For example: such as changes in N-terminal neurotensin concentration, plasma noradrenaline concentration, plasma insulin concentration. In certain embodiments, the acute increase in body mass due to the intake of food (or any substance in general can be measured by sensor(s) 54 as features for regression model 206. In certain embodiments, changes in pH (such as the “alkaline tide”) can be used.

In certain embodiments, processing circuitry 56 implements techniques to account for the effects of another aspect or aspects of the user's physiology and/or environment (for example, physical exertion, stress levels (e.g. acute mental stress has known effects on the pulse profile: (VLACHOPOULOS, C et al., 2006)), circadian rhythm, quality of the previous night's sleep, time of day, environmental temperature, ambient light levels, air quality, humidity levels, air pressure or altitude, geographical location, and/or positioning of sensor(s) (e.g. wrist, fore-arm, or upper arm), in order to maximize accuracy when calculating caloric intake of the user and/or other nutrition-related metrics. For example, output from sensor(s) 54 measuring these confounding factors (or correlates thereof) can be used as features in regression model 206, e.g. sampled at specific times (e.g. every “beat”, or every about 5 minutes) relative to the start of a given meal. For example, (NICHOLS, W et al., 2011) provides some background on certain physiological and environmental parameters (besides the desired nutrition-related metrics) known to affect the pulse profile.

As mentioned above, in certain embodiments, processing circuitry 56 can implement techniques to account for the effects of demographics in order to maximize accuracy when calculating caloric intake of the user or other nutrition-related metric(s). For example, some background on the dependence of age and the pulse profile are provided in (WANG, S H et al., 2009), and background on the dependence of disease status and the pulse profile are provided in (NICHOLS, W et al., 2011b).

In further detail, features derived from input data received at processing circuitry 56 from sensors 54 and/or features other than those directly related to physiology can be used by processing circuitry 56 (e.g. as features in the regression model 206), either in combination with or in lieu of the physiological features (such as those described herein), in order to maximize accuracy when calculating caloric intake of the user and/or other nutrition-related metrics. For example, the features based on an accelerometer can be used (such as specific bands of the power spectral density of the accelerometer data). For example, Activity Detection techniques (for example, based on accelerometer 54) can be applied to calculate the posture of the user (ATALLAH, L et al., 2010) (e.g. sitting, standing, lying (e.g. prone, supine, right side, left side)), and this posture variable(s) used as a feature(s) for regression model 206. (Example motivation: certain physiological features are known to be dependent on posture and/or movement (or behaviours correlated with posture/movement, e.g. sleep) (VRACHATIS, D et al., 2014) (YIM, Y K et al., 2014) (WANG, W K et al., 1992), thus posture information can facilitate regression model 206 in accounting for this effect. Alternatively, user activity is known to be on its own correlated with caloric intake of a meal, for example in terms of activity related to ingesting a meal (DONG, Y, 2012), or in terms of the effect of a meal on subsequent activity and psychological state (WELLS, A S et al., 1998).) As another example, the time of day (for example, minutes elapsed since about 5:00 am), season or time of year, and/or the environmental temperature (e.g. as measured by a temperature sensor, or as determined from a local weather report, for example) can be used as features. (Example motivation: certain physiological features are known to be dependent on environmental parameters such as temperature (HSIU, H et al., 2012) (HUANG, C M et al., 2011) and/or time of day (e.g. via the circadian rhythm (PORTALUPPI, F et al., 2012), thus such temperature and/or time of day information can facilitate regression model 206 in accounting for these effects; alternatively, environmental parameters are known to be correlated on their own with the nutritional content of a meal, e.g. (BOSTON, R C et al., 2008)). In certain embodiments, one or more “smell” sensor(s) and/or gas analysis sensor(s) 54 can be used to provide features for regression model 206 (e.g. informative of the presence and/or type of food or other ingested substance), for example an electronic nose.

In certain embodiments, instead of being provided as features, the confounding effects that are known to affect physiology (such as demographics, posture, environmental temperature, state of exercise, state of stress, state of sleep, having an earlier meal that occurred recently, the state of having fasted overnight or not (or having fasted in general), etc.) can be accounted for by adjusting the input features to regression model 206. As a particular example, where a pulse profile is used, a given confounding factor can be accounted for by adding a certain harmonic spectrum (e.g. known from laboratory measurements to account for the confounding factor, or by measurements obtained automatically by portable device 50, e.g. as an average of the effect all the occurrences of the confounding factor as previously detected by portable device 50) to the harmonic proportions; similarly in lieu or in combination with this additive correction, a given confounding factor can be corrected for by multiplying the harmonic proportions by a factor (e.g. known from laboratory measurements to account for the confounding factor, etc.) which is equivalent to a “transfer function”. As a further example, the additive and/or multiplicative spectrum corrections can be applied to the general Fourier spectrum domain (e.g. where the Fourier window is a pre-set time length which is not fixed to the start and/or end points of beat(s)) instead of the special case of the harmonics spectrum domain (e.g. where the Fourier window is fixed to the start and/or end points of beat(s)); the equivalent time-domain operations can also be used (e.g. time-domain convolution instead of Fourier-domain multiplication).

In certain embodiments, one or more features (e.g. used in regression model 206) can be calculated from other features (such as those described herein). For example, given the harmonic amplitudes, the distortion factor can be calculated by processing circuitry 56 according to:

d=RMS(a₂,a₃, . . . , a_{N})/a₁, (13)

Where: d is the harmonic distortion factor, RMS(•) is the Root-Mean-Square operator, aₙ is the n^{th} harmonic amplitude (or alternatively, n^{th} harmonic proportion can be used in place of aₙ). The harmonic amplitudes can be per-beat, or per average (e.g. of all the valid beats) in a given window of time e.g. about 1 minute.

For example, in one embodiment, processing circuitry 56 can calculate as a feature for regression model 206 the Incremental Area Under the Curve (IAUC) for a certain parameter or feature, e.g. blood glucose concentration, blood triglycerides concentration, HRV, cardiac output (including as calculated by Equation 12), or a linear combination of the harmonic proportions and/or phases, etc. The IAUC can be calculated by processing circuitry 56 as the integration (or the corresponding discrete summation) of the input variable, after subtracting the pre-meal baseline value, from the approximate start time of the meal until a pre-set time after the meal start (e.g. about 4 hours after the meal start). FIG. 17 provides an exemplary IAUC calculation (as the hatched region(s)), where in this example, negative excursions below the baseline are excluded. In one embodiment, multiple IAUC calculations can be combined (e.g. as a weighted average) to calculate a given nutrition-related metric with improved accuracy.

For example, in one embodiment, post-meal features can be normalized by the corresponding pre-meal features. For example, in the case of harmonic proportions, the post-meal harmonic proportions can be normalized by processing circuitry 56 according to Equation 14 and then used as features (e.g. for regression model 206):

Δhpₙ(tᵢ)=(hpₙ(tᵢ)−hpₙ(t₀))/hpₙ(t₀), (14)

Where: Δhpₙ(tᵢ) is the normalized harmonic proportion at time tᵢ; tᵢ is the time index referenced to the meal start (where t₀ is the meal start); and hpₙ(tᵢ) is the harmonic proportion (e.g. as described above) at time tᵢ. For example, Equation 14 can be applied to any feature, with the feature substituted for hpₙ. For example, hpₙ(t₀) can be taken as an average, e.g. an average over the about 30 minutes before the meal start.

In certain embodiments, one or more features (e.g. used in regression model 206) can be nutrition-related metrics, for example those described herein. For example, the one or more nutrition-related metrics to be used as features can have dedicated processing chains (e.g. which can have their own regression model 206 distinct from the regression model 206 used to predict the final nutrition-related metric(s)). For example, intake of caffeine and/or alcohol is known to affect physiology, and thus metrics describing the intake of caffeine and/or alcohol can be used as features. Other examples include the intake of spices, condiments, supplements, drugs, and/or medications, status of digestion (e.g. bloated and/or constipated), time distribution of eating (e.g. whether or not majority of ingestion for a meal took place within about 20 minutes). In addition, the processing chain(s) can be used to predict as features metrics which may not be directly nutrition-related, for example posture (e.g. standing, sitting, or lying), mental and/or emotional state, or illness (e.g. having a cold or infection, diagnosing a disease). In certain embodiments, the one or features (e.g. as input for regression model 206) for predicting nutrition-related metrics can include one or more features reflective of the previous meal history. For example, the time from the last meal and/or the nutrition-related metric(s) (e.g. caloric intake) of the last meal can be used as features; the state of having fasted overnight or not (or having fasted in general) can be used as a feature. In addition, in certain embodiments, in retrospect, features reflective of meals that occurred after the meal to be predicted can be used as features for predicting nutrition-related metrics for the earlier meal, for example, the time of the following meal and/or the nutrition-related metric(s) (e.g. caloric intake) of the following meal. In the case where a dependence loop occurs (e.g. nutrition-related metric(s) for meal 1 depends on nutrition-related metric(s) for meal 2, which depends on nutrition-related metric(s) for meal 1), for example, the processing chain can be applied iteratively until a convergence is reached (for example, the prediction of nutrition-related metric(s) are not significantly changing between iterations); in this case the machine learning algorithm used during the training process can also be adapted accordingly during its own prediction step.

During the training process, feature selection can be performed in order to select the most relevant features from a set of candidate features for use in the regression model 206 (GUYON, I et al., 2003). Feature selection algorithms are available to automate some or all of the process, but typically feature selection is performed with manual oversight by an operator who is skilled in the art. For example, univariate feature selection (e.g. using an F-test) or multivariate (e.g. L1-based feature selection such as Randomize LASSO) can be used, with an exemplary practical implementation available at (scikit-learn developers, 2014).

Calculations without Regression Model

Besides use as features in regression model 206, physiological parameters (such as those described herein) can be used by processing circuitry 56 to calculate nutrition-related parameters “directly” (e.g. without using a regression model 206 obtained via machine learning), either in combination with or in lieu of parameters calculated using regression model 206.

For example, in one embodiment, given a glycemic index and a glycemic load (e.g. from a glycemic index and glycemic load sensor(s) 54), mass of carbohydrates intake can be calculated as:

m_{carbohydrates}=GI/GL*100, (15)

Where: m_{carbohydrates} is the mass of carbohydrates intake [grams], GL is the glycemic load, and GI is the glycemic index, and the corresponding calories due to carbohydrate intake [kcal] can be calculated as C_{carbohydrates=}4*m_{carbohydrates}.

For example, in one embodiment, Equation 6 can be re-arranged in order to calculate caloric intake, given a metabolic expenditure (e.g. from a metabolic sensor 54) according to:

Cᵢₙₜₐₖₑ=C_{expenditure}+(M_{end}−Mₛₜₐᵣₜ)*K, (16)

Where: Cᵢₙₜₐₖₑ is the total caloric intake [kcal], C_{expenditure} is the total caloric expenditure [kcal], Mₛₜₐᵣₜ is the user's body mass at the start of the given window [lbs], M_{end} is the user's body mass at the end of the given window, and K is a constant (for example about 3555 [kcal/lb]). For example, M_{end} and Mₛₜₐᵣₜ (or the difference, M_{end}−Mₛₜₐᵣₜ) can be manually entered by the user (e.g. using user interface 58 and/or an application run on an external device such as a mobile phone and/or website) or automatically obtained by portable monitoring device 50 (e.g. using one or more physiological sensors 54 and/or by using a network-connected weight-scale).

For example, in one embodiment, hydration can be calculated from bioimpedance sensor 54 data via the use of spectral analysis (e.g. “bioimpedance spectroscopy”), with exemplary methods provided in U.S. Pat. No. 8,374,688, entitled “System and methods for wireless body fluid monitoring”, Libbus, I and Bly, M.

As an additional example, exemplary methods for calculating caloric content from physiological sensor(s) 54 (such as sensors measuring the Thermic Effect of Food as mentioned above) are provided in U.S. patent application Ser. No. 14/083,404, entitled “Systems and methods of measuring caloric consumption”, Teller, E et al. Additionally, exemplary methods are provided in U.S. Pat. No. 8,157,731, entitled “Method and apparatus for auto journaling of continuous or discrete body states utilizing physiological and/or contextual parameters”, Teller, E et al., in particular the text from column 70 line 18 through to column 71 line 13, which are incorporated herein by reference.

It should also be noted that any calculations that model the time evolution (such as pre-ingestion effects (e.g. food preparation activities), ingestion effects (e.g. gestures (e.g. hand-to-mouth gestures), biting, chewing, swallowing), post-prandial effects (e.g. physiological changes, clean-up activities) of variables (such as the sensor variables and/or features disclosed herein) can be used by processing circuitry 56, and these calculations do not necessarily involve use of machine learning methods such as a regression model 206.

For example, the time-pooled windows (e.g. W1, W2) described above with reference to FIG. 14 implicitly capture certain time evolution before, during, and/or following ingestion of a meal. Another example is the Incremental Area Under the Curve calculation described above. For example, in certain embodiments, parametric methods and/or Functional Data Analysis can be used to model the time evolution of one or more features and/or sensor variables; for example (FROSLIE K F et al., 2013).

Learned Features and Convolutional Neural Network

In certain embodiments, the functionality of the feature-specific calculations (block 212 of FIG. 10) can be implemented by processing circuitry 56 via execution of a machine learning model (including as part of regression model 206), an approach known in the art as “feature learning” or “representation learning” (BENGIO, Y et al., 2013). These learned features can be used either in lieu of or in combination with any manually specified features (such as those disclosed herein), as determined by what minimizes the validation error during the training process, for example.

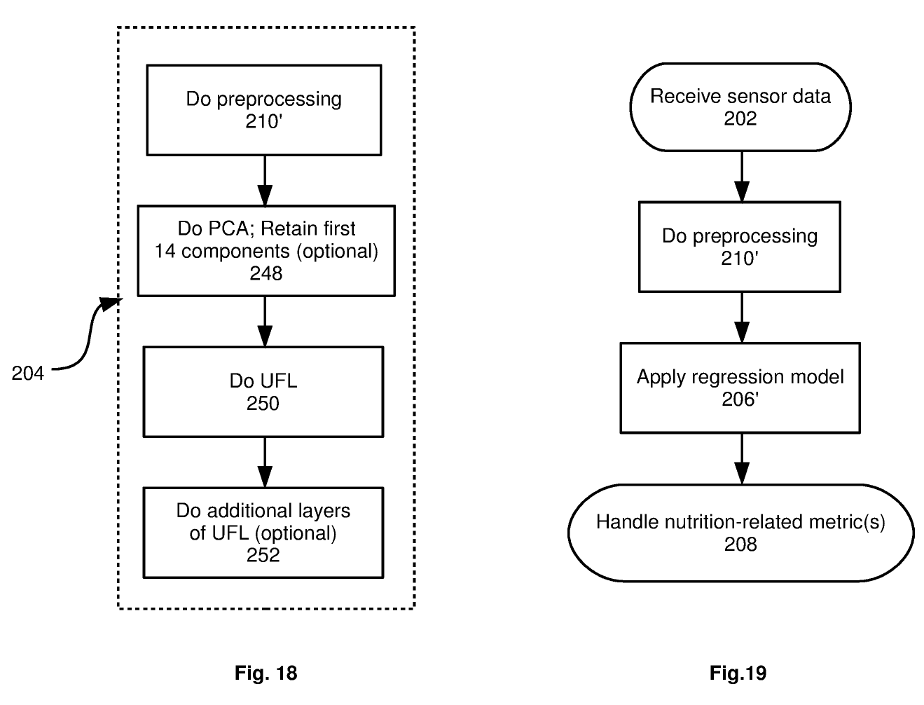

In one embodiment, the feature-specific calculation(s) 212 can be determined by an Unsupervised Feature Learning (UFL) algorithm such as Principle Components Analysis (PCA), Independent Components Analysis (ICA), Predictive Sparse Decomposition (PSD), Sparse Coding, Spike-and-Slab Sparse Coding (S3C), Auto-Encoders (including Sparse Auto-Encoders, De-noising Auto-Encoders, Contractive Auto-Encoders), or Restrictive Boltzmann Machines (RBMs); all of these examples are described in (BENGIO, Y et al., 2013). At present, PSD is the preferred UFL algorithm (KAVUKCUOGLU, K et al., 2010). For example, the input variables to feature learning can be the samples for a single beat period on the pulse profile (e.g. as indicated in FIG. 8), after period normalization 244 and amplitude normalization 246 (i.e. using the preprocessing 210′ with reference to FIG. 16), or a pre-set window of time, for example about 1.3 seconds (e.g. chosen to be longer than the majority of beat periods under standard conditions) aligned to the start of each beat, after amplitude normalization (i.e. by skipping the period normalization 244 in FIG. 16). In this embodiment, it can be advantageous to perform a whitening and/or dimensionality reduction step before the feature learning algorithm in order to reduce computations during training and making predictions and/or reduce the required training dataset size. For example, ZCA (Zero-phase Components Analysis) whitening can be used. For example, Principle Components Analysis (PCA) can be used, where only the first 14 components are retained to be used as input into the feature learning algorithm (e.g. PSD). Furthermore, in order to improve prediction accuracy, the feature learning algorithm (e.g. PSD) can be stacked in layers, where the input to a given feature learning layer is the output of the previous feature learning layer (known in the art as “greedy layer-wise training”, with the resulting model known as a “Deep Belief Network” (DBN), e.g. see (HINTON, G, DENG, L et al., 2012)). Optionally, if the UFL algorithm is stacked in layers, standardization (e.g. Z-score standardization) and/or whitening (e.g. ZCA whitening or PCA) can be applied to the input of each feature learning layer. An exemplary embodiment is illustrated in FIG. 18.

In certain embodiments, with reference to FIG. 19, the functionality of the feature-specific calculations step (block 212 of FIG. 10) and regression model 206 can be implemented together in a single regression model 206′ step. (Note: discussions in this specification regarding regression model 206 also apply to regression model 206′, according to the context of the discussions.) For example, in an embodiment, a Convolutional Neural Network (CNN) with a 1-dimensional convolutional kernel (LECUN, Y et al., 1998) (ZHENG, Y et al., 2014) can be used for regression model 206′. Indeed, one skilled in the art recognizes that a CNN is a generalization of the architecture of the feature-specific calculations 212 (e.g. blocks 214 and 216 of FIG. 11), and regression model 206 of FIG. 9, for example where instead of pre-set features (e.g. harmonic coefficients per each beat), the feature calculations 214 are replaced by learned features (i.e. neurons) (replicated in time as before, e.g. per each beat) in the input layer, the time-pooling 216 can occur with overlapping windows, and the network can have more than one layer of learned features (i.e. neurons) and/or time-pooling. Preferably, the time-pooling operation is a max operation, and the neuron activation function is a Rectified Linear Unit (ReLU). In one embodiment, the preprocessing 210′ (FIG. 16) includes period normalization (block 244 of FIG. 16), and the input convolutional kernels are aligned to the start and end of each beat. In another embodiment, the preprocessing does not include period normalization (skipping the processing in block 244 of FIG. 16), and the input convolutional kernels are aligned to the start of each beat. In another embodiment, the input convolutional kernels do not need to be aligned to the beats.

The advantage of the embodiments described in the previous paragraph (e.g. replacing feature-specific calculations 212 and regression model 206 with a CNN as regression model 206′) is an improved prediction performance, at the cost of having more parameters to fit (hence requiring a larger training dataset), increased computation (during training and/or in some cases, predictions), and having more hyperparameters to tune. The CNN can be trained in a supervised manner, for example by the use of gradient descent with back-propagation. It can be advantageous (due to improved prediction performance) to use dropout regularization (HINTON, GE, SRIVASTAVA, N et al., 2012) in the final fully-connected layer(s). As is the case with applying UFL in FIG. 18, whitening and/or dimensionality reduction can be applied as additional preprocessing in order to reduce computational requirements and reduce the required training set size (e.g. ZCA whitening, or PCA, or PCA where only the first 14 components are retained). The required training dataset size can be reduced by the use of unsupervised pre-training, for example preferably with a Predictive Sparse Decomposition CNN (PSD-CNN) (LECUN, Y et al., 2010), optionally followed by supervised fine-tuning. Furthermore, semi-supervised pre-training can be used instead of unsupervised pre-training in order to obtain even better predictive performance for a given training dataset size, for example using “Non-Parametrically Guided Auto-encoder” (NPGA) (SNOEK, J et al., 2012) or “Prior supervised Convolutional Stacked Auto-encoder” (PCSA) (WANG, Z et al., 2013) (WANG, Z et al., 2012) techniques. In order to counteract the increased computation requirements during training, it is advantageous to use training software optimized for large matrix multiplications, and designed to leverage Graphical Processing Units (GPUs), for example, the Torch7 package from (COLLOBERT, R et al., 2011). In order to determine effective settings for the relatively large number of hyperparameters, Bayesian optimization of the hyperparameters can be applied (SNOEK, J et al., 2012b) (where the validation error is being minimized), as described above, in order to avoid the need for excessive experimentation. For example, in (BERGSTRA, J et al., 2013b), Bayesian optimization (via the hyperopt package (BERGSTRA, J et al., 2013)) was applied to a CNN model family with 238 hyperparameters, and was able to find state-of-the-art settings (on a computer vision task) after about 150 iterations of configuration updates (as in block 256 of FIG. 15). For example, a range for hyperparameters optimization which include setting(s) which are approximately equivalent in the configuration of the “neurons” to the time-pooled harmonic features described above can be used as a starting point for the CNN (or CNN-PSD, etc.), for example with an input pulse profile (after preprocessing (e.g. PCA or ZCA), or in another embodiment, without any preprocessing) and an input window for a given meal from about 30 minutes before meal start to about 260 minutes after meal start, with a single time-pooling layer on the input containing around 10 convolutional features (learned “feature maps”) with convolution windows that are around 30 or 45 or 60 minutes wide and with no or minimal overlap, followed by a standard supervised regression model (e.g. SVR model, or “fully-connected” FFNN layer(s) with linear activation function output, etc.). In applying hyperparameter optimization, the range of hyperparameters and/or model configurations that can be practically considered are limited mainly by computation power and time.

In certain embodiments where frequency-based features (e.g. Fourier transform or time-frequency transforms) are used as input to the CNN 206′, a 2-dimensional convolutional kernel can be used, where the convolution happens both in time (e.g. replicated for each beat, and aligned to the start of each beat) and in frequency (e.g. for Fourier transform coefficients, 1^{st} coefficient, 2^{nd} coefficient, 9^{th} coefficient, etc.).

In certain embodiments where UFL and/or CNN are used for regression model 206, one or more additional preprocessing step(s) can be added to those shown in FIG. 16. For example, a differentiation step (or finite difference step) can be appended to the steps shown in FIG. 16. As another example, the differentiation (or finite difference) can be of the n^{th} order, where n is a positive integer, for example 1^{st} or 2^{nd} order. Furthermore, more than one set of features resulting from more than one order of differentiation can be used in combination as features input into regression model 206. In addition, the preprocessed signal with differentiation applied can be combined with the preprocessed signal without differentiation applied as a set of features input into regression model 206.

In certain embodiments where the pulse profile over a window spanning a single beat (or a window (e.g. about 1.3 s) aligned to the start of a beat) is used as input to further calculations (e.g. feature calculations, or UFL, or CNN, etc.), the “beat” or window can be replaced by an average of multiple beats or windows, e.g. all the valid beats or windows in a moving window (distinct from the “window” mentioned earlier in this paragraph; e.g. the previous about 1 minute), after period normalization.

In certain embodiments, instead of a CNN in the previously described embodiments, a generic supervised regression model can be used for regression model 206, e.g. SVR, but in the same way as described for a CNN (e.g. input features consisting of a single beat, or a single window aligned to the start of a given beat). For an exemplary implementation of this architecture being applied to the related problem of cocaine dose prediction (classification) from ECG, see (NATARAJAN, A et al., 2013).

In one embodiment, regression model 206 can be implemented by using UFL to learn the covariance kernel for Gaussian processes (e.g. for regression or classification, depending on the nutrition-related metric), optionally followed by supervised fine-tuning via back-propagation gradient descent (HINTON, G E et al., 2007). This embodiment can be implemented in order to leverage the strengths of Gaussian process regression (e.g. good prediction performance for a relatively small labelled dataset) combined with the advantages of UFL (e.g. effectively extracting information from the features, including from unlabelled data, to achieve better prediction performance for a given size dataset). Additionally, in one embodiment, regression model 206 can be implemented by Deep Gaussian Processes (DAMIANOU, A et al., 2013), in order to combine the advantages of UFL and Gaussian processes in a single architecture. Additionally, in one embodiment, Bayesian statistical regression methods for regression model 206 can be used in order to directly leverage an explicit model as a prior in the Bayesian model.

In certain embodiments, regression model 206 can be implemented by using any supervised machine learning method optimized for time-series prediction. For example, a Recurrent Neural Network (RNN) can be used in order to predict the nutrition-related metrics given features data, e.g. with a 1-dimension input for the pulse profile signal (or any other combination of signals or features, including those described herein). For example, a Long Short-term Memory RNN can be used, including with multiple levels of representation (i.e. “Deep Recurrent Neural Network”) (GRAVES, A et al., 2013). Indeed, any one or more methods for modelling the time evolution (such as pre-ingestion effects (e.g. food preparation activities), ingestion effects (e.g. gestures (e.g. hand-to-mouth gestures), biting, chewing, swallowing), post-prandial effects (e.g. physiological changes, clean-up activities) of variables (such as the sensor variables and/or features disclosed herein) via statistical methods can be used as regression model 206.

In certain embodiments, hybrid architectures are contemplated where manually-specified features (such as those described herein) are combined with learned features (such as those learned via UFL, including using one or more PSD or PSD-CNN layers) for use in regression model 206, for example, according to the combinations that decrease the validation error during the training process of FIG. 15.

Semi-Real-Time Feedback

In certain embodiments, the processing chain is executed once, after a whole batch of data pertaining to the last meal (or a single pre-set window of time, e.g. the last about 1 hour) is collected for example so that sufficient data characterizing the body's response to the meal has been obtained by pulse profile sensor(s) 52 and/or physiological/environmental sensor(s) 54 to make a satisfactory prediction. For example, the processing chain can be executed about 3 hours from the last meal or, with reference to FIG. 14, the processing chain can be executed about 90 minutes from the last meal, so that sufficient data to calculate features for time pooling windows W1 and W2 have been collected by portable device 50. Thus, the nutrition-related metrics are only made available to the user after this delay.

In other embodiments, the processing chain can be executed at multiple times for the prediction of a given meal, making a prediction of nutrition-related metrics available with less delay, and updating the initial prediction one or more times with potentially more accurate predictions as more data is collected. In this embodiment, a separate regression model can be trained for each updated prediction, for example, four separate regression models 206 can be trained for making a respective prediction after about 30 minutes, after about 1 hour, after about 2 hours, and after about 4.5 hours. In an embodiment, a regression model can make a prediction at about the meal start time (or shortly afterwards, e.g. after about 5 minutes) by using the data preceding the meal start (which we have found to be highly correlated with the caloric intake of the meal about to be consumed). For example, with reference to FIG. 14, features (such as the harmonic proportions) time pooled over the window W1 can be used to make an initial prediction of the meal. In certain embodiments, the sequence of predictions implementing “semi-real-time feedback” is timed in response to the user manually indicating the start of a meal. In other embodiments, “semi-real-time feedback” can be combined with auto-detection of meal start (e.g. described above). Furthermore, in certain embodiments, the auto-detection of meal start can require a delay in identifying the start of the meal (e.g. about 30 minutes from the meal start) in order to increase reliability of auto-detection, and this delay will necessary limit the response time of “semi-real-time feedback”. It is also contemplated that a hybrid scheme can be used where the separate regression models corresponding to different times with reference to the meal start are partially combined as one regression model, with the advantage of reduced computational requirements during training and/or prediction, and/or the sharing of statistical power. For example, a single FFNN (or CNN) can be used, where multiple output neurons are used for the different predictions at different times (optionally, 1 or more of the final hidden layers can also be separated into a sub-network for each output), and input neurons for which data has not arrived are simply set to zero in the inputs (or alternatively, in the activities of the input neurons).

In certain embodiments, techniques can be used reduce the computational requirements (and/or reduce response time) of regression model 206 during predictions, which can increase the efficiency of regression model 206 on performance constrained systems (e.g. a battery-powered “embedded” device 50 or mobile phone) or a system where many portable monitoring devices 50 are being served. For example, the number of computations and/or memory size for making predictions can be reduced by applying model compression (BUCILU\check{A}, C et al., 2006). Other examples such as quantization of model parameters in order to used fix-point arithmetic and/or optimizing calculations for specific hardware operations (e.g. SIMD (Single Instruction, Multiple Data) primitives for fixed-point computation that are provided by a modern x86 central processing unit) are described in (VANHOUCKE, V et al., 2011) and (XIAO, Y et al., 2014). Furthermore, special-purpose hardware architecture optimized for regression model 206 can be used, such as a Graphical Processing Unit (which excels at larger matrix multiplications) or custom-built hardware such as programmable logic, e.g. (AHN, B, 2014).

Example Data

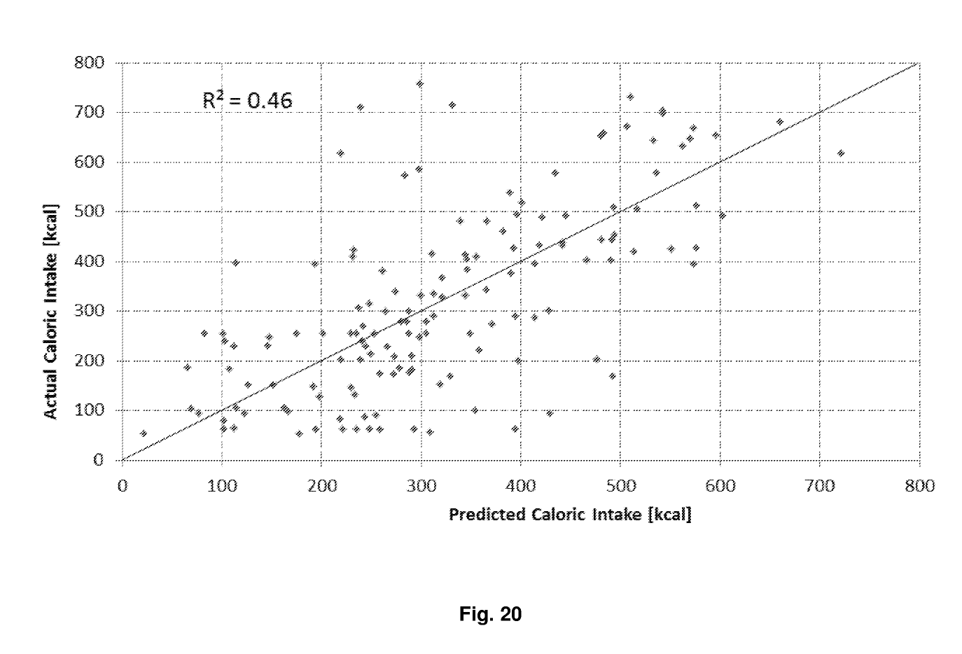

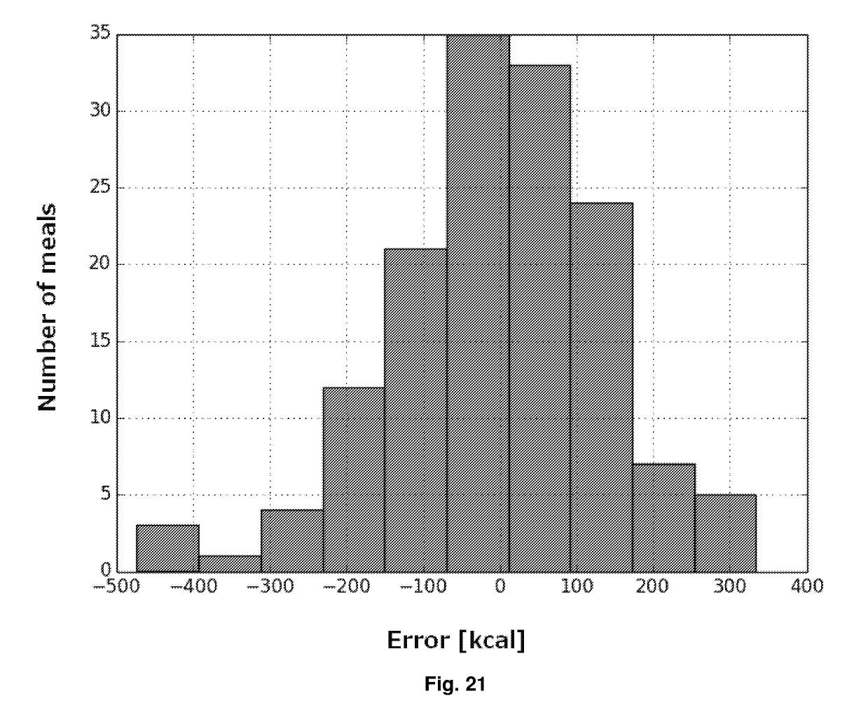

Referring now to FIG. 20 and FIG. 21, exemplary data from the preferred embodiment (i.e. PPG sensor 52 with the 24 time-pooled harmonic features (based on harmonics 1-7) described previously in reference to FIG. 11, Support Vector Regression (SVR) model for regression model 206, with a radial-basis function (RBF) kernel and hyperparameters C=3.6 and gamma=0.0525 as selected by Bayesian optimization, etc.) is presented. FIG. 20 depicts a scatter-plot showing the predicted value of caloric intake (horizontal axis) (that is, caloric intake values generated by processing circuitry 56 via the performance of the methods described above) versus the actual value of caloric intake (vertical axis), where each point is a measurement (meal). The line of perfect predictions is also shown for comparison. FIG. 21 depicts a histogram, showing the number of measurements (i.e. meals) occurring for a given amount of error in caloric intake prediction (in this case, error is the prediction minus the true value of a meal, in units of kcal). In both figures, the predictions are obtained from a validation dataset by the use of LOO-CV. The dataset consisted of 145 meals from a single user. The overall error was found to be about 106 kcal (Mean Absolute Error) with an R² coefficient of about 0.46, while the mean meal size was about 329 kcal.

For example an example of learned parameters from a regression model 206, where time-pooled harmonic amplitudes corresponding to the first 7 harmonics and time-pooling window W2 where used, pre-meal normalization according to Equation 14 was applied, and linear regression was used: w=(−9.947, 1.151, 0.986, 4.746, 0.589, −4.296, 1.109), b=329, where w is the vector of learned parameters expressed in units of kcal/% change of harmonic amplitudes from pre-meal, and b is the y-intercept or “bias” expressed in units of kcal. For an example of the typical corresponding feature values for a meal, f=(0, 17, 3, 22, −4, −21, −16), where f is the vector of feature values expressed in units of % change of harmonic amplitudes from pre-meal. For this example meal, the predicted caloric intake would be: Cᵢₙₜₐₖₑ=w*f+b=526 kcal, where * is the dot-product operator.

Additional Specification

In certain embodiments, the portable monitoring device 50 can track food sensitivity (e.g. for given meals), for example via the Coca Pulse Test.

In certain embodiments, the portable monitoring device 50 (or its variants) can track, in combination with or in lieu of nutrition-related metrics, other health-related metrics. For example, the portable monitoring device can monitor and/or calculate caloric expenditure (for example, by the use of demographic information and/or by the use of motion sensors and/or physiological sensors 54 (for example, a heart rate sensor (for example, based on a pulse profile sensor 52 such as a photoplethysmography sensor or an electrocardiography sensor))). For example, portable monitoring device 50 can monitor and/or calculate sleep-related metrics of the user (for example, hours of sleep in a given night, and/or hours of deep sleep), and/or provide for an alarm to wake the user at a specific time based on the user's circadian rhythm and/or pre-set time constraints. Portable monitoring device 50 can detect the sleep-related metrics based on one or more physiological and/or environmental sensors 54 (for example, a motion sensor, and/or a heart-rate sensor (for example, based on a pulse profile sensor 52 such as a photoplethysmography sensor, or an electrocardiography sensor), and/or a skin conductance sensor, and/or an electroencephalography sensor). For example, the pulse profile (e.g. via the harmonic proportion features) can be used by processing circuitry 56 (e.g. via a regression model 206) to predict a) the state of sleep vs. awake and/or b) the depth of sleep; for example, the pulse profile can be captured remotely (e.g. on the users bedside desk; with the advantages of not requiring the user to wear a portable device 50 during sleep and/or allowing charging (and/or syncing) of portable device 50 at night; for example, the pulse profile can be obtained via remote PPG with a light detector (e.g. a video camera) and/or light emitter (e.g. one or more LEDs) that operate in non-visible wavelengths of light e.g. infrared (with an advantage of minimally disturbing the user during sleep). For example, the portable monitoring device 50 can monitor and/or calculate stress-related metrics of the user based on data obtained by physiological and/or environmental sensor(s) 54. For example, portable monitoring device 50 can implement the stress-related metrics based on heart-rate variability derived from a heart-rate sensor (for example, based on a pulse profile sensor 52 such as a photoplethysmography sensor, or an electrocardiography sensor), and/or data from a skin conductance sensor, and/or data from an electroencephalography sensor. In certain embodiments, correlations between nutrition and/or health related metrics and/or any other metrics or context can be determined by processing circuitry 56 and presented to the user (e.g. via user interface 58 and/or a user interface on an external device such as a mobile phone or website). For example: “you sleep significantly better when you de-stress by 9:30 pm”; “you are significantly less stressed when you sleep well at night”; “you are significantly less stressed when you have a larger breakfast”.

In certain embodiments, the portable monitoring device 50 can predict metrics that are not directly nutrition-related by the use of any combination the techniques described herein for predicting nutrition-related metrics; said metrics can be output to the user and/or an external device, either combination with or in lieu of one or more nutrition-related metrics. Examples of metrics include (without limitation): the intake of drugs, and/or medications; the state and/or quality of sleep; the condition of performing certain behaviours and/or activities; posture (e.g. standing, sitting, or lying); mental and/or emotional state; health/wellness state; and illness (e.g. having a cold or infection, having an injury, diagnosing a disease).

In one embodiment, the portable monitoring device 50 can calculate as nutrition-related metrics Weight Watchers points; for example, these can be calculated from other nutrition-related metrics (such as those described; these can be calculated using techniques described herein).

In certain embodiments, the “user” need not be a single individual, in particular, the “wearer” and “operator” can be separate individuals, for example in the case of a child, less abled, and/or ill “wearer”. Furthermore, the user (or “wearer”) in the embodiments of the present specification need not be a human; indeed the physiological, behavioural, and/or environmental effects (such as many of those disclosed herein e.g. cardiovascular effects, gastric activity, autonomic effects, biting/chewing/swallowing activities, etc.) are known to apply to non-human animals and organisms.

In one embodiment, the portable monitoring device 50 can augment and/or replace calculations for nutrition-related metrics, using data from manual entry (e.g. via user interface 58 or an application run on an external device such as a mobile phone and/or website), for example to replace missing predictions of nutrition-related metrics, and/or to improve the accuracy of predictions of nutrition-related metrics (e.g. by averaging the manually entered value with the predicted value), and/or to provide additional context to the nutrition-related metrics to be displayed to the user (e.g. as in a “food journal”; e.g. via user interface 58 and/or via a user interface on an external device such as a mobile phone and/or website) alongside nutrition-related metrics e.g. to aid review of the user's eating habits. For example photo(s) (and/or video(s)) of a given meal can be used to provide the user context of said meal, and the photos can be acquired by a camera (not shown) built-in to portable device 50 and/or by a camera on an external device such as a mobile phone. Furthermore, a photo(s) of a given meal can be used by an image recognition algorithm (and/or a panel of human “experts” who give an estimation based on the photo) in order to estimate nutrition-related metrics (e.g. caloric content, serving size, and/or phytonutrient index, etc.) for a given meal to be used in lieu of or in combination with (e.g. by averaging) the automatic prediction(s) of the nutrition-related metric(s). For example, for a given food item (which can be part of a meal or the whole meal), scans (e.g. taken by a camera built-in to portable device 50, or a camera on an external device) of a product bar code (e.g. UPC code) can be used in combination with pre-existing nutritional databases in order to lookup the per-serving nutrition-related metrics (e.g. calories per serving, e.g. calories per 100 g of food), and the user can optionally specify a serving size consumed in order to enable portable device 50 to calculate the nutrition-related metrics for the given food item. Furthermore, the technique of the previous sentence can be performed with a scan of the “Nutrition Facts” label (instead of a scan of a product bar code), where optical recognition is used to determine the per-serving nutrition-related metrics. For example the user can be given the option to fill in missing data (for example, if auto-meal detection failed to detect a meal), incorrect data, or add additional context to the data e.g. in order to aid the user in reviewing their eating habits (e.g. more detailed nutritional information for a given meal, or context such as “had dinner at {location} with {people}”); for example the added context can in the form of text (for example the text can be limited in length e.g. limited to at most 140 characters). In addition, context can be automatically acquired by portable device 50 (and/or by an application run on an external device such as a mobile phone and/or website); for example location information for a given meal (or window of time) can be acquired by use of a Global Positioning System (GPS) device (either built-in to portable device 50 or built-in to an external device such as a mobile phone); other examples include: automatically acquiring context from: a personal calendar (e.g. from Google Calendar), a Facebook account, a Twitter account (e.g. of the user or someone the user follows), a blog (e.g. of the user or someone the user follows), an email inbox, SMS messages, and/or a news outlet (e.g. key headlines from today's news and/or weather context). Additionally, a location can be used to look up additional context via a database. For example, the database can consist of user-assigned descriptions (especially for frequently recurring locations, e.g. “office”, “home”, “mom's”, or “Subway sandwiches downtown”) and/or context provided by an external service (such as the service provided by the website www.foursquare.com).

For example, previous meals can be recalled by the user and displayed on user interface 58 and/or on a user interface on an external device such a mobile phone and/or a website. For example, the recall of previous meals can be limited to the most recent meals (e.g. the 10 most recent meals, or meals occurring in the last about 2 weeks). For example, the recall of previous meals can be filtered based on nutrition-related metrics e.g. high-calorie meals or small-calorie meals or any other health-related metrics or context (e.g. time, location, and/or people context). In one embodiment, time and/or location information (manually entered or automatically determined, e.g. by use of a GPS device) can be used to recall previous meals that occurred at the given time and/or location. In certain embodiments, any combination of the techniques specified in this paragraph can be applied for recalling meals by the user.

In one embodiment, the portable monitoring device 50 can include transmitter and/or receiver circuitry 60 to communicate with an external device or service or computing system (for example, see FIG. 5 and FIG. 6). For example, the portable monitoring device 50 can communicate the energy (e.g. calories) intake calculated by processing circuitry 56 to an external user interface and/or a server hosting a website (for example, www.airohealth.com). The portable monitoring device 50 can also output raw or pseudo-raw sensor data (that is, partially processed sensor data) as well as a correlation thereof. Indeed, the portable monitoring device 50 can output other nutritional or health related metrics, including any of the metrics described herein.

The portable monitoring device 50 can include transmitter and/or receiver circuitry 60 which implements or employs any form of communication link (for example, wireless, optical, or wired) and/or protocol (for example, standard or proprietary) now known or later developed, as all forms of communications protocols are intended to fall within the scope of the present specification (for example, Bluetooth, ANT (Area Network Technology), WLAN (Wireless Local Area Network), Wi-Fi, power-line networking, all types and forms of Internet based communications, and/or SMS (Short Message Service)); all forms of communications and protocols are intended to fall within the scope of the present specification.

In one embodiment, the portable monitoring device 50 makes available data (for example raw, pseudo-raw, and/or processed) to applications that run on an external device(s) (for example including third party developed or controlled applications), and/or to applications that run on a server (for example, on a webserver hosting a web site such as www.airohealth.com). In one embodiment, nutrition-related metrics (or health metrics in general) can be presented to the user in terms of recommended intake (e.g. recommend daily intake, recommended quantity per meal (for example, recommend proportion of calories for a given meal e.g. recommend % of calories due to sugar), including according to personal characteristics/demographics, personal goals, or recommendations for the general population. For example, a given nutrition-related metric can be displayed alongside the corresponding recommended daily intake, and/or displayed as a proportion of a recommended daily intake (e.g. “the protein content of this meal represents 56% of your recommended daily intake of protein”). For example, the recommend daily intake of calories can be determined by estimating the user's caloric expenditure (C_{expenditure} described above, also known in the art as “metabolic rate”). For example, nutrition-related metrics and the corresponding recommended intake or personal goals can be summarized for a period of time beyond a single day (e.g. for a week (e.g. the last week) or for a month (e.g. the last month) and presented to the user in order to summarize their progress. For example plots (e.g. bar plots, line plots, and/or pie charts) and/or a calendar format (e.g. where information is organized chronologically and labelled by day, day of the week, week of the month, month of the year, and/or year) can be used to organize the summary.

In one embodiment, the user can set a goal(s) regarding their body weight and/or body composition (e.g. body fat percentage, lean muscle mass percentage); for example the user's body weight (and/or body composition) and their goal body weight (and/or body composition) can be summarized for the user to understand their progress towards their goal; as a further example the user's body weight (and/or body composition) can be automatically updated via network-connected scale. For example, in the case of a body composition goal, portable device 50 can have sensors 54 which measure body mass, for example using bioimpedance. For example, the bioimpedance body-composition can be measured locally (e.g. via electrodes which create an electrical circuit through the body at a particular local region such as the wrist in the case of a wrist-located portable device 50) or “globally” (e.g. via electrodes which create an electrical circuit through the body going beyond the region in the vicinity of portable device 50, a wrist-located portable device 50 can have contact the wrist on which it is worn (e.g. the left wrist) and require the user to use their free hand (e.g. right hand) to touch a second electrode, completing an electrical circuit through the arms and torso of the body).

In another embodiment, the nutrition-related metrics can be presented to the user (for example via user interface 58 and/or via a user interface on an external device such a mobile phone and/or via a website) in terms of goals that depend on time and/or location context. For example, the user can set a goal(s) to eat more meals (and/or a greater proportion of calories) earlier in the day (for example, a goal to eat breakfast more often, or a goal to eat at least 50% of one's calories before 3 p.m.). Conversely, the user can set a goal(s) to eat fewer meals (and/or a lesser proportion of calories) later in the day (for example, a goal to avoid eating meals past 9:00 p.m.). For example, the user can set a goal(s) to eat more meals (and/or a greater proportion of calories) at certain location(s) (e.g. at “home”) and/or to eat fewer meals (and/or a lesser proportion of calories) at certain location(s) (e.g. at “McDonald's” or at “the office”). Additionally, in one embodiment location data and/or data from physiological/environmental sensor(s) 54 (such as a heart-rate sensor (e.g. based on a pulse profile sensor 52) and/or a motion sensor) can be used to determine if the user is “on the go”, for example standing, walking, driving, or otherwise in transit between locations. In this embodiment, the user can set a goal(s) to eat more meals (and/or a greater proportion of calories) while not “on the go” (or to spend more minutes sitting and/or inclining for a given meal). Additionally, in one embodiment, stress-related metrics (as described above) can obtained, and the user can set a goal(s) to eat more meals while less stressed (for example, the user can attempt to minimize stressful activities around meals such as driving, having a work meeting, eating in front of a computer, or multi-tasking).

In one embodiment, the user can set a goal with regards to one or more nutritional quality metrics, and portable device 50 can present the nutritional quality metrics in terms of the user's goal (for example via user interface 58 and/or via a user interface on an external device such a mobile phone and/or via a website). For example, the nutritional quality metric can be determined as described above. For example, the user can manually rate their meals based on quality e.g. how filling and/or satiating is a given meal (e.g. on a scale from 1 to 5) and/or how one feels in response to a given meal (e.g. “energized” or “lethargic”/“heavy”/“bloated”). For example, the user can be automatically notified (for example, after opting in) by portable monitoring device 50 (and/or an external device such as a mobile phone and/or website) at a pre-set time after meal start (e.g. about 1 hour after the meal start) in order to rate a given meal; this automatic notification can be applied where the meal start was manually input or where the meal start was automatically detected.

In one embodiment, the portable monitoring device 50 can receive data from an external device (such as a mobile phone), for example in order to modify the operation of portable monitoring device 50 (for example, improve accuracy of the calculations, and/or minimize power consumption e.g. according to methods described below where portable device 50's sensor(s) or circuitry are powered off (or to a less active, lower power state) when the sensor data is unlikely to be useful (e.g. unlikely to correspond to a meal, or likely to be corrupted by motion)) and/or to give feedback to the user (for example, nutritional or other health-related metrics, advice, instructions, and/or motivational messages) and/or to receive information from the user (for example, from an external user interface such as a mobile phone application).

In one embodiment, data intended to be sent to an external device can be stored locally (using persistent or volatile storage, not shown; e.g. Flash memory (e.g. MultiMediaCard (MMC) or Secure Digital (SD) cards (including swappable or hard-wired into processing circuitry 56), embedded MMC (e-MMC)), RAM, and/or EEPROM) if the external device cannot be reached by device 50, to be sent to the external device when communications between device 50 and the external device are re-established.

In certain embodiments, where a real-time clock is used by processing circuitry (e.g. in order to record the time of meal start, quantify the time evolution of features, etc.), the time can be set automatically during synchronization (e.g. wired, wirelessly, etc) with an external device, e.g. a mobile phone, a personal computer, etc.

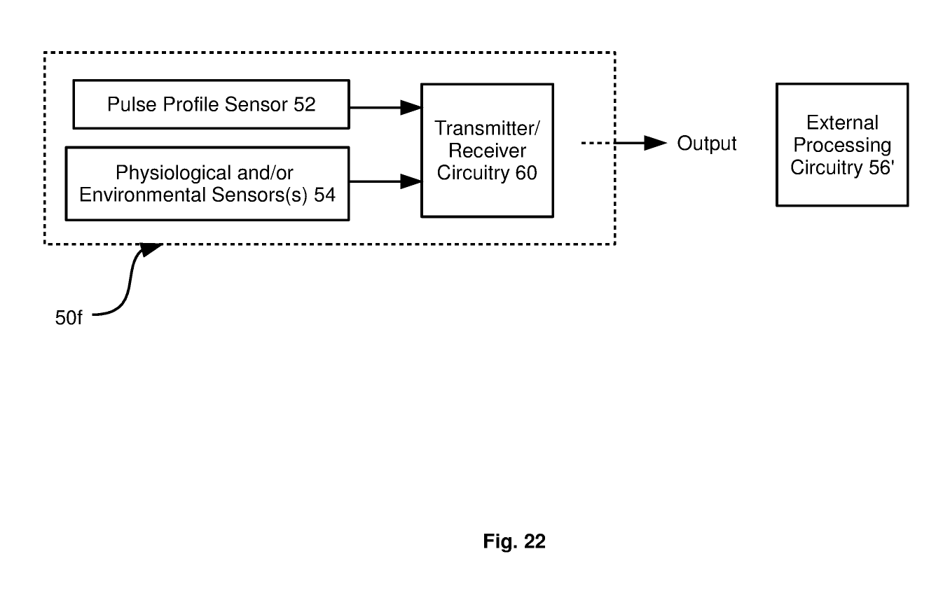

For example, in one embodiment, the portable monitoring device 50 of the present specification includes one or more pulse profile sensor(s) 52 and/or one or more physiological and/or environmental sensor(s) 54 and/or in certain embodiments other sensors. In this embodiment, the portable monitoring device 50f however, does not include processing circuitry 56 to monitor and/or calculate caloric intake (and/or other nutritional metrics) due to ingestion of food. In this embodiment, as shown in FIG. 22, processing circuitry 56′ is implemented “off-device” or external to the portable monitoring device 50f. Here, the portable monitoring device 10 can store (using persistent or volatile storage, not shown) and/or communicate (i) data which is representative of the pulse profile and/or (ii) data which is representative other physiological and/or environmental parameters to external processing circuitry 56′ (for example, on a mobile phone and/or on a server) wherein such external processing circuitry 56′ can monitor caloric intake (and/or other nutritional metrics) due to ingestion of food of the user. Such external circuitry can implement the calculation processes and techniques in near real-time or after-the-fact (e.g. in batches). The (i) data which is representative of the pulse profile and/or (ii) data which is representative other physiological and/or environmental parameters can be communicated to such external processing circuitry 56′, for example, via transmitter and/or receiver circuitry 60 (see FIG. 22), removable memory, electrical or optical communication (for example, hardwired communications via USB). Hybrid architectures are also contemplated whereby processing circuitry 56 is included in device 50f, however device 50f is configured so that some or all of the functions of processing circuitry 56 can be performed outside device 50 using external processing circuitry 56′. For example, the external processing circuitry 56′ can do the more intensive aspects of processing and/or storage in order to reduce the power and/or memory utilization of portable monitoring device 50, and processing circuitry 56 can be used to send any required sensor data (including raw or partially processed data) in an efficient (e.g. compressed and/or only when a meal has occurred/is likely to have occurred and/or only when valid sensor data has occurred/is likely to have occurred) yet timely manner, and in some variants, receive data from the external processing circuitry 56′ e.g. nutrition-related metrics, or partially processed sensor data, etc.

Moreover, the portable monitoring device 50f of FIG. 22 can include all permutations and combinations of sensors (for example, one or more pulse profile sensor(s) 52, and/or physiological and/or environmental sensor(s) 54) discussed herein.

In one embodiment, the portable monitoring device can implement measures to reduce power consumption, such as a change in sampling rate of the sensor(s), and/or a temporary power off of the sensor(s) and/or some or all of the processing circuitry 56 (and/or any other processing circuitry) and/or transmitter circuitry/receiver circuitry 60. For example, these power-saving techniques can be based on a time schedule (for example, cycling between being powered on for about one minute and being powered off for about four minutes), and/or based on an indicator of signal quality (for example, a motion sensor can indicate when the sensor data is most likely to be corrupted by motion artifacts, and thus could be ignored to reduce power consumption), and/or based on the user's state (for example, the nutritional sensors can be less active if it is determined that the user is sleeping, or if a meal is unlikely to have occurred recently e.g. the last about 4.5 hours). For example, these power-saving techniques can be automatically adapted to reduce power without excessively compromising signal quality, given changes in the conditions of operation. For example, a PPG sensor 52 can be configured to achieve a Signal-to-Noise Ratio (SNR) within a desired range. For example, where harmonic N (e.g. harmonic 7) is the highest order (and/or lowest amplitude, as the pulse profile harmonics generally decrease in amplitude with order) harmonic component of interest, the SNR can be measured by portable device 50 as the ratio between the average of amplitude of harmonics N−1 and N (“signal amplitude”) and the average (or minimum) of amplitudes of harmonics N+1 through N+5 (“noise amplitude”). Alternatively, the SNR can be measured as the ratio between harmonics N−1 and N and a pre-set amplitude representing the “noise floor” e.g. as measured during development and/or manufacturing of portable device 50. The SNR can be calculated by processing circuitry 56 on a per-beat basis, and then averaged over one or more beats (e.g. all the valid beats in an about 1 minute window), with the resulting average SNR being used as the SNR metric e.g. to adapt the power vs. noise trade-off of sensor(s) 52, 54. For example, if the SNR is above a pre-set threshold (e.g. about 2), the PPG sensor 52 can be configured by processing circuitry 56 to decrease SNR in order to save power (for example, by decreasing the LED power and/or by increasing the permitted circuit noise (e.g. by using less signal amplification of the detected optical-signal)); conversely if the SNR is too low (e.g. below a threshold e.g. about 1.5; additionally, hysteresis can be used) the PPG sensor 52 can be configured by processing circuitry to increase SNR. Example changes in conditions for which the PPG sensor 52 can automatically adapt to maintain a desired SNR/power trade-off include if the sensor coupling and/or blood circulation is changed during operation (causing an increase or decrease in pulse-profile “AC” amplitude). In another example, a user with lighter skin tone can require less LED power (or less photo-signal amplification) from a PPG sensor 52 in order to achieve the same SNR as a person with darker skin tone, and the portable device 50 can detect this and adjust the configuration of PPG sensor 52 accordingly. In certain embodiments, the same principles can be applied to any sensor(s) 52, 54 where there is a power vs. noise trade-off and a minimum SNR requirement. In certain embodiments, a regression model 206 can be trained and/or optimized for a lower SNR condition; for example the training process can be applied to the lower SNR data (e.g. from a pulse profile sensor 52; e.g. this lower SNR data can be from direct measurement or it can be emulated by adding noise such as white noise into a clean signal). As a further example, the higher (/noisier) harmonics can be ignored (e.g. ignoring #6, 7 but retaining #1-5) when they are determined by processing circuitry 56 to be dominated by noise, and a regression model 206 that was trained on data with only the lower harmonics (e.g. 1-5) retained can be applied to the lower SNR data in order to predict nutrition-related metrics.

In one embodiment, an initial meal detection algorithm is optimized to have low resource utilization and a low false negative rate, and this initial algorithm determines whether a more costly meal start calculation and/or nutrition-related metric calculation should be used. As mentioned above, compressive sensing techniques (BAHETI, P K et al., 2009) can be used in the sampling of sensors (such as pulse profile sensor 52 and/or physiological and/or environmental sensors 54) and/or processing and/or transmitting of data e.g. in order to reduce resource usage e.g. power consumption and/or memory utilization (e.g. by reduced sensor sampling, and/or reduced utilization of transmitter/receiver circuitry 60, and/or reduced utilization of processing circuitry 56, etc.) and/or perform de-noising of a signal. In certain embodiments, where a compressive sensing technique(s) applied by processing circuitry 56 requires an estimate of the sensor noise, the sensor can temporarily run at full sample rate in order to measure the noise e.g. noise amplitude. In certain embodiments signal compression techniques such as delta encoding or linear predictive coding (LPC) can be applied by processing circuitry 56 to the sensor 52, 54 data e.g. to. In one embodiment, the harmonic coefficients (e.g. complex harmonic proportions, or alternatively harmonic amplitude proportions, and phases) are used for storage and/or transmission in order to reduce resource usage e.g. processing and/or memory utilization of processing circuitry 56 an/or utilization of transmitter/receiver 60 and/or power consumption. Similarly, in one embodiment, other features can be used as a more compact form of the pulse profile for further processing in order to reduce resource usage. In one embodiment, the stored features can be down sampled e.g. pooled into about 1 minute windows in order to reduce resource usage.

The portable monitoring device 50 can include a rechargeable (or non-rechargeable) battery (not shown) or ultracapacitor to provide electrical power to the circuitry and other elements of the portable monitoring device 50. For example, lithium-ion technology, nickel-metal hydride technology, and/or aluminum-ion technology can be used in the one or more batteries. In one embodiment, the one or more energy storage elements (for example, battery or storage capacitor) can obtain energy from, for example, a charger (which can be a wireless (e.g. using electromagnetic resonance) or inductive charger) or other energy source (e.g. ambient light via solar cells, body heat, or body motion, etc.). For example, wireless charging based on one or more of the following standards can be used for power portable device 50 (including charging built-in energy storage element(s): Alliance for Wireless Power (A4WP), Power Matters Alliance (PMA), and/or Wireless Power Consortium (Qi). In certain embodiments, one or more energy storage elements can be removable, for example enabling the user to “swap” out a depleted energy storage element for a charged energy storage element; another example case would be to replace a dead storage element (e.g. battery that no longer holds a satisfactory charge). In certain embodiments, portable device 50 can be powered “directly” by an external energy source, that is, without having to charge an one or more intermediate energy storage element. In one embodiment, the power conversion circuitry (not shown) can use one or more Switching-Mode Power Supplies (SMPS).

FIG. 23 is a side perspective view of an exemplary physical configuration of portable monitoring device 50 according to an embodiment and FIG. 24 is a top perspective view of an exemplary physical configuration of portable monitoring device 50 according to the same embodiment. For example, the top section can have a thickness about 6.0 mm and a width about 22.0 mm, and the bottom section can have a thickness about 7.5 mm and a width about 15 mm at the most narrow portion.

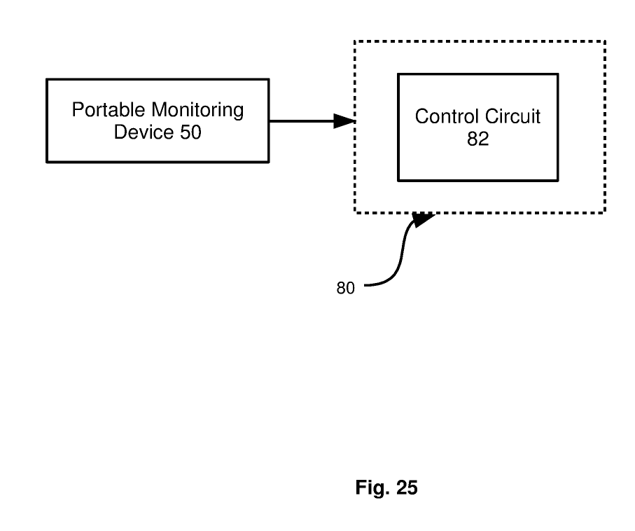

In one embodiment, with reference to FIG. 25, the portable monitoring device 50 is connected to an insulin pump 80 having a control circuit 82 being configured to meter one or more dose(s) of insulin based on the nutritional metrics provided by portable monitoring device 50. In a more particular example, the nutritional metrics can include blood glucose concentrations used by the insulin pump to meter a dose of insulin in order to reduce excessively high blood glucose concentrations and/or increase excessively low blood glucose concentrations. In another example, the nutritional metrics can include one or more of the time of the meal, the mass of carbohydrates of the meal, and glycemic index of the meal to be used by the insulin pump to meter a dose of insulin in order to counteract the anticipated effect of the meal on the blood glucose concentration. In another example, the nutritional metrics can include one or more of the time of the meal, the mass of carbohydrates of the meal, and the glycemic index of the meal to be used by the insulin pump to calculate/adjust a carbohydrates-to-insulin ratio. The carbohydrates-to-insulin ratio can be used by the insulin pump, in combination with the measured or anticipated carbohydrates of a meal, to meter a dose of insulin in order to counter the anticipated effect of the meal on the blood glucose concentration. In addition, any of the embodiments of this paragraph may be combined in an embodiment. Descriptions of an exemplary insulin pump which can be used to implement insulin pump 80 are found in Blomquist, “Carbohydrate Ratio Testing Using Frequent Blood Glucose Input,” U.S. patent application Ser. No. 11/679,712, filed Feb. 27, 2007.

While the foregoing specifically discloses certain embodiments, it is to be understood that combinations, variations and subsets of those embodiments are contemplated and will now be apparent to the person skilled in the art. For example, device 50 (and its variants) can be incorporated into medical equipment for automatically administering nutrients or medications to an individual according to an individual need that is ascertainable from the calculations made by the device. A non-limiting example of such medical equipment is an insulin pump that automatically injects insulin into an individual at times and quantities that are based on measurements made by the device.

The scope of the claims should not be limited by the embodiments set forth in the above examples, but should be given the broadest interpretation consistent with the description as a whole.

### REFERENCES

AHN, B. ‘Computation of deep belief networks using special-purpose hardware architecture’. In: 2014 International Joint Conference on Neural Networks (IJCNN), New York, N.Y.: IEEE, 2014, pages 141-148.
AMFT, O et al. On-Body Sensing Solutions for Automatic Dietary Monitoring. Pervasive Computing, IEEE, 2009, Vol. 8, No. 2, pages 62-70.
ATALLAH, L et al. ‘Sensor placement for activity detection using wearable accelerometers’. In: 2010 International Conference on Body Sensor Networks (BSN). New York. N.Y.: IEEE, 2010, pages 24-29.
AVIV, R et al. The use of gastric electrical signals for algorithm for automatic eating detection in dogs. Neurogastroenterology & Motility, Vol. 20, No. 4, pages 369-376.
AtCor Medical Pty Ltd (formerly PWV Medical Pty Ltd). Reference Values for SphygmoCor Px. Datasheet [online]. [retrieved on 2014-November-30]. Retrieved from the Internet: <URL: http://www.atcormedical.com/pdf/TN8%20-%20Reference%20Values%20for%20SphygmoCor%20Px.pdf>
BAHETI, P K et al. ‘An ultra low power pulse oximeter sensor based on compressed sensing’. In: Sixth International Workshop on Wearable and Implantable Body Sensor Networks (BSN), 2009. New York, N.Y.: IEEE, 2009, pages 144-148.
BENGIO, Y et al. Representation learning: A review and new perspectives. IEEE Transactions on Pattern Analysis and Machine Intelligence, August 2013, Vol. 35, No. 8, pages 1798-1828.
BERGSTRA, J et al. ‘Hyperopt: A python library for optimizing the hyperparameters of machine learning algorithms’. In: Proceedings of the 12th Python in Science Conferences (SciPy 2013), Edited by: S. van der Walt et al. SciPy Conferences, 2013, pages 13-20.
BERGSTRA, J et al. ‘Making a science of model search: Hyperparameter optimization in hundreds of dimensions for vision architectures’. In: JMLR Workshop and Conference Proceedings, Edited by: S. Dasgupta et al. Journal of Machine Learning Research, 2013, Vol. 28, pages 115-123.
BLANCH, N et al. Postprandial effects of potassium supplementation on vascular function and blood pressure: a randomised cross-over study. Nutrition, Metabolism and Cardiovascular Diseases, February 2014, Vol. 24, No. 2, pages 148-154.
BLANN, A D et al. Inflammation does not influence arterial stiffness and pulse-wave velocity in patients with coronary artery disease. Journal of Human Hypertension, October 2013, Vol. 27, No. 10, pages 629-634.
BOUBOULIS, P et al. Complex Support Vector Machines for Regression and Quaternary Classification [online]. arXiv preprint arXiv:1303.2184v3, 2014 [retrieved on 2015-Sep.-10]. Retrieved from the Internet: <URL: http://arxiv.org/abs/1303.2184v3>.
BOSTON, R C et al. Modeling circadian rhythms of food intake by means of parametric deconvolution: results from studies of the night eating syndrome. The American journal of clinical nutrition, June 2008, Vol. 87, No. 6, pages 1672-1677.
BREIMAN, L. Random Forests. Machine Learning, October 2001, Vol. 45, No. 1, pages 5-32.
BRUNDIN, T et al. Influence of protein ingestion on human splanchnic and whole-body oxygen consumption, blood flow, and blood temperature. Metabolism, May 1994, Vol. 43, No. 5, pages 626-632.
BUCILU\check{A}, C et al. ‘Model compression’. In: Proceedings of the 12th ACM SIGKDD international conference on Knowledge discovery and data mining. New York, N.Y.: ACM, 2006, pages 535-541.
BUTTERFIELD, Thomas. Project: Detection of heart rate by measuring impedance of the wrist [online]. 2008 [retrieved on 2014-Nov.-30]. Retrieved from the Internet: <URL: http://field4.co.uk/physmes_project.pdf>, especially FIG. 4.5 and Section 4.5.4.
CAMBRIA, E et al. Extreme Learning Machines [Trends & Controversies]. Intelligent Systems, IEEE, December 2013, Vol. 28, No. 6, pages 30-59.
CHANG, C C et al. LIBSVM: A library for support vector machines. ACM Transactions on Intelligent Systems and Technology (TIST), April 2011, Vol. 2, No. 3, ArticleID 27, 27 pages.
COLLOBERT, R et al. ‘Torch7: A matlab-like environment for machine learning’. In: BigLearn, NIPS Workshop. Big Learning, 2011. No. EPFL-CONF-192376.
DAMIANOU, A et al. ‘Deep Gaussian Processes’. In: JMLR Workshop and Conference Proceedings. Edited by: C. M. Carvalho et al. Journal of Machine Learning Research, 2013, Vol. 31, pages 207-215.
DICKINSON, K M et al. Postprandial effects of a high salt meal on serum sodium, arterial stiffness, markers of nitric oxide production and markers of endothelial function. Atherosclerosis, January 2014, Vol. 232, No. 1, pages 211-216.
DEHGHAN, M et al., Is bioelectrical impedance accurate for use in large epidemiological studies? Nutrition Journal, Vol. 7, No. 26. <DOI: 10.1186/1475-2891-7-26>.
DONG, B et al. Wearable sensing for liquid intake monitoring via apnea detection in breathing signals. Biomedical Engineering Letters, 2014, Vol. 4, No. 4, pages 378-387.
DONG, Y. Tracking wrist motion to detect and measure the eating intake of free-living humans [online]. PhD diss., Graduate School of Clemson University, 2012 [retrieved on 2014-Nov.-30]. Retrieved from the Internet: <URL: http://www.ces.clemson.edu/˜ahoover/theses/dong-diss.pdf>.
DRAPER, N R et al. Applied regression analysis. 3rd ed. New York, N.Y.: John Wiley and Sons, 1998, ISBN: 978-0-471-17082-2. 736 pages.
ELGENDI, M. On the analysis of fingertip photoplethysmogram signals. Current cardiology reviews, February 2012, Vol. 8, No. 1, pages 14-25.
ERHAN, D et al. ‘Scalable Object Detection Using Deep Neural Networks’. In: 2014 IEEE Conference on Computer Vision and Pattern Recognition (CVPR). New York, N.Y.: IEEE, 2014, pages 2155-2162.
ESSER, D et al. A high-fat SFA, MUFA, or n3 PUFA challenge affects the vascular response and initiates an activated state of cellular adherence in lean and obese middle-aged men. The Journal of Nutrition, June 2013, Vol. 143, No. 6, pages 843-851.
FRANCESCHINI, M A et al. Near-infrared spiroximetry: noninvasive measurements of venous saturation in piglets and human subjects. Journal of Applied Physiology, January 2002, Vol. 92, No. 1, pages 372-384.
FROSLIE K F et al. Shape information from glucose curves: Functional data analysis compared with traditional summary measures. BMC Medical Research Methodology, Vol. 13, No. 6. <DOI: 10.1186/1471-2288-13-6>.
FORSYTH, D A et al. Computer Vision: A Modern Approach [online]. 2nd edition, Upper Saddle River, N.J.: Prentice Hall, 2011 [retrieved on 2014-11-30]. Retrieved from the Internet: <URL: http://luthuli.cs.uiuc.edu/˜daf/CV2E-site/detectingextracts.pdf> Chapter 17, ISBN-10: 013608592X.
GHISELLI, A et al. Total antioxidant capacity as a tool to assess redox status: critical view and experimental data. Free Radical Biology and Medicine, December 2000, Vol. 29, No. 11, pages 1106-1114.
GIRSHICK, R et al. Rich feature hierarchies for accurate object detection and semantic segmentation [online]. arXiv preprint arXiv:1311.2524v5, 2014 [retrieved on 2014-Nov.-30]. Retrieved from the Internet: <URL: http://arxiv.org/abs/1311.2524v5>.
GLAROS, K N. Low-power pulse oximetry and transimpedance amplifiers [online]. PhD diss., Imperial College London Department of Bioengineering, October 2011 [retrieved on 2015-09-10]. Retrieved from the Internet: <URL: http://www.bg.ic.ac.uk/research/e.drakakis/downloadFiLES/Glaros_PhD_Thesis.pdf>.
GOLDMAN, J M et al. Masimo Signal Extraction Pulse Oximetry. Journal of Clinical Monitoring and Computing, September 2000, Vol. 16, No. 7, pages 475-483.
GONLACHANVIT, S et al. Effect of meal size and test duration on gastric emptying and gastric myoelectrical activity as determined with simultaneous [13C]octanoate breath test and electrogastrography in normal subjects using a muffin meal. Digestive diseases and sciences, December 2001, Vol. 46, No. 12, pages 2643-2650.
GRAVES, A et al. ‘Speech recognition with deep recurrent neural networks’. In: 2013 IEEE International Conference on Acoustics, Speech and Signal Processing (ICASSP). New York, N.Y.: IEEE, 2013. pages 6645-6649.
GRAYBEAL, J M et al. ‘Adaptive Filtering and Alternative Calculations Revolutionizes Pulse Oximetry Sensitivity and Specificity During Motion and Low Perfusion’. In: Proceedings of the 26th Annual International Conference of the IEEE Engineering in Medicine and Biology Society (EMBS). New York, N.Y.: IEEE, 2004, pages 5364-5366.
GU, C et al. ‘Recognition using regions’. In: 2009 IEEE Conference on Computer Vision and Pattern Recognition (CVPR). New York, N.Y.: IEEE, 2009, pages 1030-1037.
GUYON, I et al. ‘An introduction to variable and feature selection’. In: JMLR Workshop and Conference Proceedings. Edited by: I. Guyon et al. Journal of Machine Learning Research, 2003, Vol. 3, pages 1157-1182.
HAWLEY, S K et al. Relative effects of fat-, carbohydrate- and protein-containing liquid diets on cardiac output in healthy adult subjects. Clinical Science, October 1992, Vol. 83, No. 4, pages 483-487.
HAYKIN, S. Neural Networks: A Comprehensive Foundation. 2nd ed. Upper Saddle River, N J: Prentice Hall, 1998, ISBN: 0132733501. 842 pages.
HINTON, G E et al. ‘Using Deep Belief Nets to Learn Covariance Kernels for Gaussian Processes’. In: Advances in Neural Information Processing Systems 20 (NIPS 2007). Edited by: J. C. Platt et al. La Jolla, Calif.: Neural Information Processing Systems Foundation, 2007, pages 1249-1256.
HINTON, G E et al. Improving neural networks by preventing co-adaptation of feature detectors [online]. arXiv preprint arXiv:1207.0580v1, 2012 [retrieved on 2014-11-30]. Retrieved from the Internet: <URL: http://arxiv.org/abs/1207.0580v1>.
HINTON, G et al. Deep neural networks for acoustic modeling in speech recognition: The shared views of four research groups. Signal Processing Magazine, IEEE, November 2012, Vol. 29, No. 6, pages 82-97, especially FIG. 1.
HOFFMAN, R P et al. Hyperglycemia without hyperinsulinemia produces both sympathetic neural activation and vasodilation in normal humans. Journal of Diabetes and its Complications, January-February 1999, Vol. 13, No. 1, pages 17-22.
HOGAS, S et al. Changes in arterial stiffness following dialysis in relation to overhydration and to endothelial function. International urology and nephrology, June 2012, Vol. 44, No. 3, pages 897-905.
HOLMER-JENSEN, J et al. Acute differential effects of dietary protein quality on postprandial lipemia in obese non-diabetic subjects. Nutrition Research, January 2003, Vol. 33, No. 1, pages 34-40.
HOLZER, P. Physiology of the Gastrointestinal Tract, Two Volume Set [online]. Fifth edition. Waltham, Mass.: Academic Press, 2012 [retrieved on 2015-09-10]. Retrieved from the Internet: <URL: https://books.google.ca/books?id=ytWBLAy1FGQC&pg=PA832> <DOI: 10.1016/B978-0-12-382026-6.00029-4> Chapter 29, page 832-845.
HSIU, H et al. Effects of Cold Stimulation on the Harmonic Structure of the Blood Pressure and Photoplethysmography Waveforms. Photomedicine and Laser Surgery, February 2012, Vol. 30, No. 2, pages 77-84.
HSU, T L et al. Similarity Between Coffee Effects and Qi-Stimulating Events. The Journal of Alternative and Complementary Medicine, November 2008, Vol. 14, No. 9, pages 1145-1150.
HUANG, C M et al. Radial Pressure Pulse and Heart Rate Variability in Heat- and Cold-Stressed Humans. Evidence-Based Complementary and Alternative Medicine, 2011, Vol. 2011, ArticleID 751317, 9 pages.
HUANG, G B et al. Extreme learning machine: theory and applications. Neurocomputing, November-December 2006, Vol. 70, No. 1, pages 489-501.
HUBER, W A (username: “whuber”). ‘Analysis with complex data, anything different?’. In: Cross Validated [online]. New York, N.Y.: Stack Exchange, Oct. 4, 2013; 21:11:36Z [retrieved on 2014-11-30]. Retrieved from the Internet: <URL: http://stats.stackexchange.com/questions/66088/analysis-with-complex-data-anything-different/66268#66268>.
IANDOLA, F et al. DenseNet: Implementing Efficient ConvNet Descriptor Pyramids [online]. arXiv preprint arXiv:1404.1869v1, 2014 [retrieved on 2014-11-30]. Retrieved from the Internet: <URL: http://arxiv.org/abs/1404.1869v1>.
JENKINS, A et al. Acute Response of Equicaloric Test Meals Varying in Glycemic Index and Glycemic Load on Postprandial Glycemia, Arterial Stiffness and Blood Pressure in Healthy Adults. The FASEB Journal, April 2010, Vol. 24, Meeting Abstracts, abstract No. 324.8.
KALS, J et al. Inflammation and oxidative stress are associated differently with endothelial function and arterial stiffness in healthy subjects and in patients with atherosclerosis. Scandinavian Journal of Clinical & Laboratory Investigation, 2008, Vol. 68, No. 7, pages 594-601.
KAVUKCUOGLU, K et al. Fast Inference in Sparse Coding Algorithms with Applications to Object Recognition [online]. arXiv preprint arXiv:1010.3467v1, 2010 [retrieved on 2014-Nov.-30]. Retrieved from the Internet: <URL: http://arxiv.org/abs/1010.3467v1>.
KEARNEY, M T et al. The cardiovascular responses to feeding in man. Experimental Physiology, September 1995, Vol. 80, No. 5, pages 683-700.
KHOR, A et al. Postprandial oxidative stress is increased after a phytonutrient-poor food but not after a kilojoule-matched phytonutrient-rich food. Nutrition Research, May 2014, Vol. 34, No. 5, pages 391-400.
KULCU, E et al. Physiological differences between interstitial glucose and blood glucose measured in human subjects. Diabetes Care, August 2003, Vol. 26, No. 8, pages 2405-2409.
KUO, C D et al. The effects of high-fat and high-carbohydrate diet loads on gas exchange and ventilation in COPD patients and normal subjects. CHEST Journal, July 1993, Vol. 104, No. 1, pages 189-196.
KVIETYS, P R. The Gastrointestinal Circulation [online]. San Rafael, Calif.: Morgan & Claypool Life Sciences, 2010 [retrieved on 2015-Sep.-10]. Retrieved from the Internet: <URL: http://www.ncbi.nlm.nih.gov/books/NBK53092/> Chapter 5.
KWAN, B C et al. Bioimpedance spectroscopy for the detection of fluid overload in Chinese peritoneal dialysis patients. Peritoneal Dialysis International, June 2014, Vol. 34, No. 4, pages 409-416.
LAGUNA, J O et al. ‘A Dynamic Sliding Window Approach for Activity Recognition’. In: UMAP'11 Proceedings of the 19th international conference on User modeling, adaption, and personalization. Edited by J A Konstan et al. New York, N.Y.: Springer-Verlag Berlin Heidelberg, 2011, pages 219-230.
LECUN, Y et al. ‘Convolutional Networks for Images, Speech, and Time-Series’. In: The handbook of brain theory and neural networks. Cambridge, Mass.: MIT Press, 1998, pages 255-258.
LECUN, Y et al. ‘Convolutional networks and applications in vision’. In: Proceedings of 2010 IEEE International Symposium on Circuits and Systems (ISCAS). New York, N.Y.: IEEE, 2010, pages 253-256.
LENNON-EDWARDS, S et al. Lower potassium intake is associated with increased wave reflection in young healthy adults. Nutrition journal, April 2014, Vol. 13, No. 1, ArticleID 39, 8 pages.
LIPSITZ, L A et al. Hemodynamic and autonomic nervous system responses to mixed meal ingestion in healthy young and old subjects and dysautonomic patients with postprandial hypotension. Circulation, February 1993, Vol. 87, No. 2, pages 391-400.
LITHANDER, F E et al. Postprandial effect of dietary fat quantity and quality on arterial stiffness and wave reflection: a randomised controlled trial. Nutrition Journal, July 2013, Vol. 12, ArticleID 93, 8 pages.
LIU, Y P et al. Central systolic augmentation indexes and urinary sodium in a white population. American journal of hypertension, January 2013, Vol. 26, No. 1, pages 95-103.
LIVESEY, G. A perspective on food energy standards for nutrition labelling. British Journal of Nutrition, March 2001, Vol. 85, No. 3, pages 271-287.
MAATEN, L et al. ‘Learning with marginalized corrupted features’. In: JMLR Workshop and Conference Proceedings, Edited by: S. Dasgupta et al. Journal of Machine Learning Research, 2013, Vol. 28, pages 410-418.
MAI-MUD, A et al. Divergent effect of acute and chronic alcohol on arterial stiffness. American journal of hypertension, March 2002, Vol. 15, No. 3, pages 240-243.
MCCARTY, M F. Proposal for a dietary “phytochemical index”. Medical hypotheses, 2004, Vol. 63, No. 5, pages 813-817.
MCCUE, M D. ‘Specific dynamic action: A century of investigation’. In: Comparative Biochemistry and Physiology Part A: Molecular & Integrative Physiology, August 2006, Vol. 144, No. 4, pages 381-394.
MEREDITH, D J et al. Photoplethysmographic derivation of respiratory rate: a review of relevant physiology. Journal of medical engineering & technology, January 2012, Vol. 36, No. 1, pages 1-7.
MILLIS, R M et al. Metabolic Energy Correlates of Heart Rate Variability Spectral Power Associated with a 900-Calorie Challenge. Journal of Nutrition and Metabolism, 2011, Vol. 2011, ArticleID 715361, 6 pages.
MONTE-MORENO, E. Non-invasive estimate of blood glucose and blood pressure from a photoplethysmograph by means of machine learning techniques. Artificial intelligence in medicine, October 2011, Vol. 53, No. 2, pages 127-138.
MyFitnessPal, Inc. Free Calorie Counter, Diet & Exercise Journal [online]. San Francisco, Calif.: MyFitnessPal, 2014 [retrieved on 2014-November-30]. Retrieved from the Internet: <URL: http://www.myfitnesspal.com/>.
NATARAJAN, A et al. ‘Detecting cocaine use with wearable electrocardiogram sensors’. In: Proceedings of the 2013 ACM international joint conference on Pervasive and ubiquitous computing. New York, N.Y.: ACM, 2013, pages. 123-132.
NESTEL, P J et al. Post-prandial remnant lipids impair arterial compliance. Journal of the American College of Cardiology, June 2001, Vol. 37, No. 7, pages 1929-1935.
NEUHOUSER, M L et al. Development of a Glycemic Index Database for Food Frequency Questionnaires Used in Epidemiologic Studies. The Journal of Nutrition, June 2006, Vol. 136, No. 6, pages 1604-1609.
NICHOLS, W et al. McDonald's Blood Flow in Arteries, Sixth Edition: Theoretical, Experimental and Clinical Principles. Boca Raton, Fla.: CRC Press, 2011, ISBN-10: 0340985011. chapter 27 and chapter 29.
NICHOLS, W et al. McDonald's Blood Flow in Arteries, Sixth Edition: Theoretical, Experimental and Clinical Principles. Boca Raton, Fla.: CRC Press, 2011, ISBN-10: 0340985011. chaper 20, chapter 21, chapter 23, chapter 24, and chapter 25.
PARINI, P et al. Lipoprotein profiles in plasma and interstitial fluid analyzed with an automated gel-filtration system. European Journal of Clinical Investigation, February 2006, Vol. 36, No. 2, pages 98-104.
PAULO, L M. Automatic detection and classification of food intake [online]. PhD diss., Clarkson University, 2010 [retrieved on 2015-Sep.-10]. Retrieved from the Internet: <URL: http://gradworks.umi.com/34/06/3406718.html>.
PERKIÖMÄKI, J S et al. Fractal and complexity measures of heart rate variability. Clinical and experimental hypertension, February-April 2005, Vol. 27, No. 2-3, pages 149-158.
PHILLIPS, L K et al. The Effect of a High-Fat Meal on Postprandial Arterial Stiffness in Men with Obesity and Type 2 Diabetes. The Journal of Clinical Endocrinology & Metabolism, September 2010, Vol. 95, No. 9, pages 4455-4459.
PHUA, C T. Novel method of blood pulse and flow measurement using the disturbance created by blood flowing through a localized magnetic field [online]. PhD diss., Université Paris-Est, 2012 [retrieved on 2015-September 10]. Retrieved from the Internet: <URL: https://tel.archives-ouvertes.fr/tel-00794495/document>. English. <NNT: 2012PEST1099>. <tel-00794495>
PORTALUPPI, F et al. Circadian rhythms and cardiovascular health. Sleep medicine reviews, April 2012, Vol. 16, No. 2, pages 151-166.
RASMUSSEN, C E et al. Gaussian Processes for Machine Learning [online]. Cambridge, Mass.: MIT Press, 2006 [retrieved on 2014-Nov.-30]. Retrieved from the Internet: <URL: http://www.gaussianprocess.org/gpm1/chapters/RW2.pdf> Chapter 2, ISBN-10 026218253X.
RECIO-RODRIGUEZ, J I et al. Glycemic index, glycemic load, and pulse wave reflection in adults. Nutrition, Metabolism and Cardiovascular Diseases, September 2014, doi: 10.1016/j.numecd.2014.08.007, 7 pages.
REDDY, K A et al. Use of Fourier Series Analysis for Motion Artifact Reduction and Data Compression of Photoplethysmographic Signals. IEEE Transactions on Instrumentation and Measurement, May 2009, Vol. 58, No. 5, pages 1706-1711.
RIEZZO, G et al. Electrogastrography in Adults and Children: The Strength, Pitfalls, and Clinical Significance of the Cutaneous Recording of the Gastric Electrical Activity. BioMed research international, 2013, Vol. 2013, ArticleID 282757, 14 pages.
RIFKIN, R M et al. Notes on Regularized Least Squares. MIT Computer Science and Artificial Intelligence Laboratory Technical Report MIT-CSAIL-TR-2007-025 [serial online], [retrieved on 2014-Nov.-30]. Retrieved from the Internet <URL: http://cbcl.mit.edu/publications/ps/MIT-CSAIL-TR-2007-025.pdf>.
scikit-learn developers. ‘1.11. Feature selection’. In: scikit-learn User Guide, 2014 [online]. [retrieved on 2014-Nov.-30]. Retrieved from the Internet: <URL: http://scikit-learn.org/stable/modules/feature_selection.html>.
SHAMMAS, N. Least-Squares Relative Error Regression [online]. [retrieved on 2015-Sep.-10]. Retrieved from the Internet: <URL: http://www.namirshammas.com/HP39gII/HP39gII_part4.pdf>.
SHIMIZU, Y et al. ‘Theoretical base of alcoholic-intake detection using blood-pulse signals and new findings’. In: 2010 IEEE Sensors. Edited by: E. Katz et al. New York, N.Y.: IEEE, 2010, pages 1216-1221.
SNOEK, J et al. ‘On Nonparametric Guidance for Learning Autoencoder Representations’. In:
JMLR Workshop and Conference Proceedings. Edited by: N. Lawrence et al. Journal of Machine Learning Research, 2012, Vol. 22, pages 1073-1080.
SNOEK, J et al. ‘Practical Bayesian optimization of machine learning algorithms’. In: Advances in Neural Information Processing Systems 25 (NIPS 2012), Edited by: F. Pereira et al. La Jolla, Calif.: Neural Information Processing Systems Foundation, 2012, pages 2951-2959.
SNOEK, J (username: “JasperSnoek”). spearmint [online]. San Francisco, Calif.: GitHub, Oct. 31, 2014 [retrieved on 2014-November 30]. Retrieved from the Internet: <URL: https://github.com/JasperSnoek/spearmint>.
SUN, Y et al. Noncontact imaging photoplethysmography to effectively access pulse rate variability. Journal of biomedical optics, June 2013, Vol. 18, No. 6, ArticleID 061205, 9 pages.
TAMURA, T et al. Wearable Photoplethysmographic Sensors—Past and Present. Electronics, June 2014, Vol. 3, No. 2, pages 282-302.
VAN BAAK, M A. Meal-induced activation of the sympathetic nervous system and its cardiovascular and thermogenic effects in man. Physiology & behavior, May 2008, Vol. 94, No. 2, pages 178-186.
VANHOUCKE, V et al. ‘Improving the speed of neural networks on CPUs’. In: Proc. NIPS 2011 Deep Learning and Unsupervised Feature Learning Workshop. La Jolla, Calif.: Neural Information Processing Systems Foundation, 2011.
VLACHOPOULOS, C et al. Acute Systemic Inflammation Increases Arterial Stiffness and Decreases Wave Reflections in Healthy Individuals. Circulation, October 2005, Vol. 112, No. 14, pages 2193-2200.
VLACHOPOULOS, C et al. Acute Mental Stress Has a Prolonged Unfavorable Effect on Arterial Stiffness and Wave Reflections. Psychosomatic medicine, March-April 2006, Vol. 68, No. 2, pages 231-237.
VON BORELL, E et al. Heart rate variability as a measure of autonomic regulation of cardiac activity for assessing stress and welfare in farm animals—A review. Physiology & Behaviour, Vol. 92, No. 3, pages 293-316.
VRACHATIS, D et al. Effect of supine versus sitting position on noninvasive assessment of aortic pressure waveform: a randomized cross-over study. Journal of Human Hypertension, April 2014, Vol. 28, No. 4, pages 236-241.
WAALER, B A et al. The effect of meal size on postprandial increase in cardiac output. Acta Physiologica Scandinavica, May 1991, Vol. 142, No. 1, pages 33-39.
WAALER, B A, et al. Post-prandial cardiovascular responses in man after ingestion of carbohydrate, protein or fat. Acta Physiologica Scandinavica, December 1992, Vol. 146, No. 3, pages 321-327.
WANG, J et al. Development of forearm impedance plethysmography for the minimally invasive monitoring of cardiac pumping function. Journal of Biomedical Science and Engineering, February 2011, Vol. 4, No. 2, pages 122-129.
WANG, L et al. ‘Noninvasive cardiac output estimation using a novel photoplethysmogram index’. In: 2009 Annual International Conference of the IEEE Engineering in Medicine and Biology Society (EMBC). Edited by: Z. P. Liang et al. New York, N.Y.: IEEE, 2009, pages 1746-1749.
WANG, S H et al. ‘Age-related changes in specific harmonic indices of pressure pulse waveform’. In: 13th International Conference on Biomedical Engineering. Edited by: C. T. Lim et al. New York, N.Y.: Springer Berlin Heidelberg, 2009, Vol. 23, pages 183-185.
WANG, W K et al. Study on the pulse spectrum change before deep sleep and its possible relation to EEG. Chinese Journal of Medical and Biological Engineering, March 1992, Vol. 12, No. 1, pages 107-115.
WANG, W K et al. The prandial effect on the pulse spectrum. The American Journal of Chinese Medicine (World Scientific), 1996, Vol. 24, No. 1, pages 93-98.
WANG, Z et al. ‘Learning with target prior’. In: Advances in Neural Information Processing Systems 25 (NIPS 2012). Edited by: F. Pereira et al. La Jolla, Calif.: Neural Information Processing Systems Foundation, 2012, pages 2231-2239.
WANG, Z et al. ‘Deep feature learning using target priors with applications in ECoG signal decoding for BCI’. In: Proceedings of the Twenty-Third International Joint Conference on Artificial Intelligence. Edited by: F. Rossi. Palo Alto, Calif.: AAAI Press, 2013, pages 1785-1791.
WELLS, A S et al. Effects of carbohydrate and lipid on resting energy expenditure, heart rate, sleepiness, and mood. Physiology & behavior, February 1998, Vol. 63, No. 4, pages 621-628.
WESTERTERP, K R et al. Diet induced thermogenesis. Nutrition & Metabolism, August 2004, Vol. 1, No. 1, ArticlelD 5, 5 pages.
XIAO, Y et al. Speeding up deep neural network based speech recognition systems. Journal of Software, October 2014, Vol. 9, No. 10, pages 2706-2712.
YACIN, S M et al. ‘Pulse Rate Variability and Gastric Electric Power in Fasting and Postprandial Conditions’. In: 2009 Annual International Conference of the IEEE Engineering in Medicine and Biology Society (EMBC). Edited by Z. P. Liang. New York, N.Y.: IEEE, 2009, pages 2639-2642.
YACIN, S M et al. On non-invasive measurement of gastric motility from finger photoplethysmographic signal. Annals of biomedical engineering, December 2010, Vol. 38, No. 12, pages 3744-3755.
YAN, Y S et al. An Efficient Motion-Resistant Method for Wearable Pulse Oximeter. IEEE Transactions on Information Technology in Biomedicine, May 2008, Vol. 12, No. 3, pages 399-405.
YIM, Y K et al. The Effect of Food Intakes on Radial Pulse Amplitude. Korean Journal of Acupuncture, January 2011, Vol. 28, No. 2, pages 13-22.
YIM, Y K et al. The Effect of Short-term Bed-rest on Radial Pulse in Healthy Subjects. Journal of Acupuncture and Meridian Studies, June 2014, Vol. 7, No. 3, pages 133-139.
ZALOSZYC, A et al. Hydration measurement by bioimpedance spectroscopy and blood pressure management in children on hemodialysis. Pediatric Nephrology, November 2013, Vol. 28, No. 11, pages 2169-2177.
ZHENG, Y et al. ‘Time Series Classification Using Multi-Channels Deep Convolutional Neural Networks’. In: Web-Age Information Management. Edited by: F. Li et al. New York, N.Y.: Springer International Publishing, 2014, pages 298-310.
ZIMMERMANN, H G, et al. ‘Comparison of the Complex Valued and Real Valued Neural Networks Trained with Gradient Descent and Random Search Algorithms’. In: European Symposium on Artificial Neural Networks. Evere, Belgium: ESANN, 2011, pages 213-218.

1. A system for monitoring nutritional intake, comprising:
a housing;
a biosensor supported by the housing for disposition adjacent to a blood vessel of a user; the biosensor configured to collect pulse profile data;
an output device; and
a processing circuit connected to the biosensor and the output device; the processing circuit configured to:
receive the pulse profile data from the biosensor as a time-varying signal;
decompose the time-varying signal into a plurality of vector components representing the time-varying signal;
based on the plurality of vector components, generate a nutritional intake value from the received pulse profile data; and
control an output device to output the nutritional intake value.

2. The system of claim 1, wherein the output device includes a display.

3. The system of claim 1, wherein the output device is supported by the housing.

4. The system of claim 1, wherein the processing circuit is supported by the housing.

5. The system of claim 1, further comprising:
a transmitter-receiver circuit connected to the biosensor and configured to send the pulse profile data to an external device housing the processing circuit.

6. The system of claim 1, wherein the housing comprises a bracelet.

7. The system of claim 1, wherein the biosensor includes a photoplethysmography (PPG) sensor.

8. The system of claim 1, the processing circuit further configured to:
select a subset of the pulse profile data covering a time period; and
generate the nutritional intake value from the vector components corresponding to the subset of the pulse profile data.

9. The system of claim 8, the processing circuit further configured to:
select the subset of the pulse profile data by:
detecting a meal start time representing a beginning of ingestion of food by the user; and
selecting a beginning of the time period and an end of the time period based on the meal start time.

10. The system of claim 9, the processing circuit further configured to detect the meal start time by selecting one of a meal classification and a non-meal classification for the pulse profile data based on preconfigured classification parameters.

11. The system of claim 1, the processing circuit further configured to generate the nutritional intake value by:
retrieving regression model parameters defining a relationship between the vector components and the nutritional intake value; and
applying the regression model parameters to the vector components.

12. The system of claim 11, the processor further configured to:
prior to retrieving the regression model parameters, retrieve a training data set and select the regression model parameters based on the training data set by executing a regression model learning process.

13. The system of claim 11, the processor further configured to generate the nutritional intake value based on the frequency components and at least one additional feature derived from the pulse profile data.

14. The system of claim 13, the processing circuit further configured to automatically identify the at least one additional feature by executing a feature learning process.

15. The system of claim 11, wherein each of the plurality of vector components is a harmonic of the pulse profile data.

16. The system of claim 1, wherein the nutritional intake value is a caloric intake value.

17. The system of claim 1, the processing circuit further configured to generate an additional nutritional intake value.
